(19) United States
(12) Patent Application Publication
(10) Pub. No.: US 2026/0234277 A1
(43) Pub. Date: Aug. 13, 2026

(54) TREATMENT OF RECURRENT PERICARDITIS BY DELIVERY OF ANTI-INTERLEUKIN-1 RECEPTOR ANTIBODY

(71) Applicant: Kiniksa Pharmaceuticals, GmbH, Zug (CH)

(72) Inventors: John Paolini, Lexington, MA (US); Eben Tessari, Lexington, MA (US)

(21) Appl. No.: 19/532,470

(22) Filed: Feb. 6, 2026

Related U.S. Application Data
(60) Provisional application No. 63/755,981, filed on Feb. 7, 2025.
(60) Provisional application No. 63/761,351, filed on Feb. 21, 2025.
(60) Provisional application No. 63/785,412, filed on Apr. 8, 2025.
(60) Provisional application No. 63/810,394, filed on May 22, 2025.
(60) Provisional application No. 63/883,451, filed on Sep. 17, 2025.

(51) Int. Cl. C07K 16/28; A61K 39/00; A61P 9/00; A61P 29/00

(52) U.S. Cl. CPC C07K 16/2866; A61P 9/00; A61P 29/00; A61K 2039/505; A61K 2039/545; C07K 2317/52; C07K 2317/565; C07K 2317/76

(57) ABSTRACT

The present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control.

### CROSS-REFERENCE TO RELATED APPLICATIONS

This application claims priority to and the benefit of U.S. Provisional Application No. 63/755,981, filed Feb. 7, 2025, U.S. Provisional Application No. 63/761,351, filed Feb. 21, 2025, U.S. Provisional Application No. 63/785,412, filed Apr. 8, 2025, U.S. Provisional Application No. 63/810,394, filed May 22, 2025, and U.S. Provisional Application No. 63/883,451, filed Sep. 17, 2025; the disclosure of each of which is hereby incorporated by reference in its entirety.

### INCORPORATION-BY-REFERENCE OF SEQUENCE LISTING

This application contains a sequence listing filed electronically in XML format. The present application hereby incorporates by reference the entire contents of the XML file named “KPL-062US1_SL.xml”, which was created on Feb. 5, 2026, and is 59,481 bytes in size.

### BACKGROUND

Recurrent pericarditis (RP) is a rare autoinflammatory disease which affects 20% to 30% of patients after an incident acute pericarditis event (Imazio 2014) and can be debilitating due to pain and limitations in physical function. RP is characterized by an initial acute pericarditis event followed by one or more subsequent acute recurrences after a symptom-free interval of 4-6 weeks (Adler 2015). Pericarditis recurrences increase rates of emergency room admissions and hospitalizations. In the United States, 5% of patients presenting to the emergency room with non-ischemic chest pain are diagnosed with pericarditis (Agarwal 2015).

Currently available treatments for pericarditis or recurrent pericarditis include medications to reduce the inflammation and swelling associated with pericarditis. Commonly used medications are steroids, such as colchicine. Colchicine can reduce the duration of pericarditis symptoms and decrease the risk that the condition will recur, but the medication is not safe for patients with pre-existing health conditions like liver or kidney disease or for patients taking certain medications and may cause side effects, including nausea and diarrhea, that can lead to discontinuation of treatment. Steroids are known to cause significant side effects, particularly with long-term use.

### SUMMARY OF THE INVENTION

The present invention provides, among other things, a convenient, more tolerable, and safer method for treating recurrent pericarditis (RP) based on an anti-IL1R1 antibody using dosing regimens described herein. The present invention is, in part, based on a therapeutically effective dosing regimen resulting in (1) a statistically-significant drop on a Numerical Rating Scale (NRS) for assessment of pericarditis pain; (2) normalization of C-Reactive Protein level for assessment of pericarditis inflammation; (3) low recurrence rate; (4) rapid treatment response; and/or (5) rapid time to monotherapy (e.g., successful taper of background medications, concurrent therapies or standard of care).

In one aspect, the present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control.

In some embodiments, the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In some embodiments, the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose sufficient to improve, stabilize, prevent or reduce one or more symptoms of recurrent pericarditis relative to a control, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In some embodiments, a HCDR1, a HCDR2, a HCDR3, LCDR1, a LCDR2, and a LCDR3 are determined by Kabat numbering system. In some embodiments, a HCDR1, a HCDR2, a HCDR3, LCDR1, a LCDR2, and a LCDR3 are determined by Chothia numbering system. In some embodiments, a HCDR1, a HCDR2, a HCDR3, LCDR1, a LCDR2, and a LCDR3 are determined by IMGT numbering system.

In some embodiments, the one or more symptoms of recurrent pericarditis are assessed by a Numerical Rating Scale (NRS) for assessment of pericarditis pain. In some embodiments, the one or more symptoms of recurrent pericarditis are assessed by an echocardiogram (ECG). In some embodiments, the one or more symptoms of recurrent pericarditis are assessed by cardiac magnetic resonance imaging (MRI). In some embodiments, the one or more symptoms of recurrent pericarditis are assessed by a Quality of Life Questionnaire, or a portion thereof (e.g., SF-36 Health Survey, or Patient Global Impression of Pericarditis Symptom Severity (PGIPS)).

In some embodiments, the one or more symptoms of recurrent pericarditis comprise fever. In some embodiments, the one or more symptoms of recurrent pericarditis comprise pericardial rub. In some embodiments, the one or more symptoms of recurrent pericarditis comprise an elevated level of C-reactive protein (CRP). In some embodiments, the one or more symptoms of recurrent pericarditis comprise chest pain. In some embodiments, the one or more symptoms of recurrent pericarditis comprise shortness of breath. In some embodiments, the one or more symptoms of recurrent pericarditis comprise heart palpitations. In some embodiments, the one or more symptoms of recurrent pericarditis comprise swelling. In some embodiments, the one or more symptoms of recurrent pericarditis is fatigue. In some embodiments, the one or more symptoms of recurrent pericarditis is the inability to exercise.

In one aspect, the present invention provides, among other things, a method of treating recurrent pericarditis comprising administering subcutaneously to a patient an anti-interleukin-1 receptor type-1 (IL1R1) antibody at a dose of 300 mg, wherein the anti-IL1R1 antibody is administered once every two weeks or once every four weeks.

In one aspect, the present invention provides, among other things, a method of treating recurrent pericarditis comprising administering subcutaneously to a patient an anti-interleukin-1 receptor type-1 (IL1R1) antibody at a dose of 300 mg, wherein the anti-IL1R1 antibody is administered once every two weeks or once every four weeks, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis or reducing the risk of a pericarditis recurrence comprising administering subcutaneously to a patient an anti-interleukin-1 receptor type-1 (IL1R1) antibody at a dose of 300 mg, wherein the anti-IL1R1 antibody is administered once every two weeks or once every four weeks.

In one aspect, the present invention provides, among other things, a method of treating recurrent pericarditis or reducing the risk of a pericarditis recurrence comprising administering subcutaneously to a patient an anti-interleukin-1 receptor type-1 (IL1R1) antibody at a dose of 300 mg, wherein the anti-IL1R1 antibody is administered once every two weeks or once every four weeks, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering subcutaneously to a patient an anti-interleukin-1 receptor type-1 (IL1R1) antibody at a dose of 100 mg once every two weeks or once every four weeks.

In one aspect, the present invention provides, among other things, a method of treating recurrent pericarditis comprising administering subcutaneously to a patient an anti-interleukin-1 receptor type-1 (IL1R1) antibody at a dose of 100 mg once every two weeks or once every four weeks, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis or reducing the risk of a pericarditis recurrence comprising administering subcutaneously to a patient an anti-interleukin-1 receptor type-1 (IL1R1) antibody at a dose of 100 mg once every two weeks or once every four weeks.

In one aspect, the present invention provides, among other things, a method of treating recurrent pericarditis or reducing the risk of a pericarditis recurrence comprising administering subcutaneously to a patient an anti-interleukin-1 receptor type-1 (IL1R1) antibody at a dose of 100 mg once every two weeks or once every four weeks, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the patient has recurrence after a symptom-free interval. In some embodiments, the patient does not have a symptom-free interval.

In one aspect, the present invention provides, among other things, a method of treating recurrent pericarditis comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement.

In one aspect, the present invention provides, among other things, a method of treating recurrent pericarditis comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement, without a symptom-free interval.

In one aspect, the present invention provides, among other things, a method of treating recurrent pericarditis comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of is persisting signs and symptoms of pericarditis despite initial improvement, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of reducing symptoms or risk of recurrent pericarditis comprising administering to a patient, an anti-interleukin-1 receptor type-1 (IL1R1) antibody at a therapeutically effective dose.

In one aspect, the present invention provides, among other things, a method of reducing symptoms or risk of recurrent pericarditis comprising administering to a patient, an anti-interleukin-1 receptor type 1 (IL1R1) antibody at a therapeutically effective dose, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of reducing symptoms or risk of recurrent pericarditis comprising administering subcutaneously to a patient an anti-interleukin-1 receptor type-1 (IL1R1) antibody at a dose of 300 mg, wherein the anti-IL1R1 antibody is administered once every two weeks or once every four weeks.

In one aspect, the present invention provides, among other things, a method of reducing symptoms or risk of recurrent pericarditis comprising administering subcutaneously to a patient an anti-interleukin-1 receptor type-1 (IL1R1) antibody at a dose of 300 mg, wherein the anti-IL1R1 antibody is administered once every two weeks or once every four weeks and the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides among other things, a method of reducing symptoms or risk of recurrent pericarditis comprising administering subcutaneously to a patient an anti-interleukin-1 receptor type-1 (IL1R1) antibody at a dose of 100 mg once every two weeks or once every four weeks.

In one aspect, the present invention provides among other things, a method of reducing symptoms or risk of recurrent pericarditis comprising administering subcutaneously to a patient an anti-interleukin-1 receptor type-1 (IL1R1) antibody at a dose of 100 mg once every two weeks or once every four weeks, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of reducing symptoms or risk of recurrent pericarditis comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement.

In one aspect, the present invention provides, among other things, a method of reducing symptoms or risk of recurrent pericarditis comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method for the treatment of idiopathic recurrent pericarditis or reduction in risk of recurrence comprising administering to a patient, an anti-interleukin-1 receptor type-1 (IL1R1) antibody at a therapeutically effective dose.

In one aspect, the present invention provides, among other things, a method for the treatment of idiopathic recurrent pericarditis or reduction in risk of recurrence comprising administering to a patient, an anti-interleukin-1 receptor type-1 (IL1R1) antibody at a therapeutically effective dose, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method for the treatment of idiopathic recurrent pericarditis or reduction in risk of recurrence comprising administering subcutaneously to a patient an anti-interleukin-1 receptor type-1 (IL1R1) antibody at a dose of 300 mg, wherein the anti-IL1R1 antibody is administered once every two weeks or once every four weeks.

In one aspect, the present invention provides, among other things, a method for the treatment of idiopathic recurrent pericarditis or reduction in risk of recurrence comprising administering subcutaneously to a patient an anti-interleukin-1 receptor type-1 (IL1R1) antibody at a dose of 100 mg once every two weeks or once every four weeks.

In one aspect, the present invention provides, among other things, a method for the treatment of idiopathic recurrent pericarditis or reduction in risk of recurrence comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement.

In one aspect, the present invention provides, among other things, a method for the treatment of idiopathic recurrent pericarditis or reduction in risk of recurrence comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis or reducing the risk of a pericarditis recurrence comprising administering to a patient, an anti-interleukin-1 receptor type-1 (IL1R1) antibody at a therapeutically effective dose.

In one aspect, the present invention provides, among other things, a method of treating recurrent pericarditis or reducing the risk of a pericarditis recurrence comprising administering to a patient, an anti-interleukin-1 receptor type-1 (IL1R1) antibody at a therapeutically effective dose, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis or reducing the risk of a pericarditis recurrence comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement.

In one aspect, the present invention provides, among other things, a method of treating recurrent pericarditis or reducing the risk of a pericarditis recurrence comprising administering to a patient, an anti-interleukin-1 receptor type-1 (IL1R1) antibody, wherein the patient has at least one episode of acute pericarditis with pericarditis pain NRS ≥4 and CRP ≥1.0 mg/dL followed by a period of persisting signs and symptoms of pericarditis despite initial improvement, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 is administered at a therapeutically effective dose sufficient to improve, stabilize, prevent or reduce one or more symptoms of recurrent pericarditis relative to a control.

In some embodiments, the persisting signs and symptoms of pericarditis comprise one or more of: a) pericarditis pain; b) an elevated CRP level; c) pericardial rub; d) widespread ST segment elevation or PR segment depression according to ECG findings; e) pericardial effusion; f) pericardial inflammation; g) edema; h) evidence of pericarditis by late gadolinium enhancement; i) evidence of constriction; and j) fever, chest pain, shortness of breath, and/or heart palpitations.

In some embodiments, the persisting signs and symptoms of pericarditis do not include the combination of pericarditis pain NRS ≥4 and CRP ≥1.0 mg/dL.

In some embodiments, the initial improvement is an improvement of one or more signs or symptoms associated with an episode of acute pericarditis.

In some embodiments, the initial improvement comprises a reduction or improvement in one or more of: a) pericarditis pain; b) CRP; c) pericardial rub; d) widespread ST segment elevation or PR segment depression according to ECG findings; e) pericardial effusion; f) pericardial inflammation; g) edema; h) evidence of pericarditis by late gadolinium enhancement; i) evidence of constriction; and j) fever, chest pain, shortness of breath, and/or heart palpitations.

In some embodiments, pericarditis pain is measured by Numerical Rating Scale (NRS). In some embodiments, the initial improvement is indicated by a NRS of <4. In some embodiments, the initial improvement is indicated by a NRS of <3. In some embodiments, the initial improvement is indicated by a NRS of <2.

In some embodiments, pericardial inflammation is assessed by cardiac MRI.

In some embodiments, edema is assessed by T2 short-tau inversion recovery (STIR) imaging.

In some embodiments, the evidence of pericarditis by late gadolinium enhancement is assessed by cardiac MRI.

In some embodiments, the initial improvement is indicated by CRP <1.0 mg/dL.

In some embodiments, the period of persisting signs and symptoms of pericarditis is 1-10 weeks. In some embodiments, the period of persisting signs and symptoms of pericarditis is 2-10 weeks. In some embodiments, the period of persisting signs and symptoms of pericarditis is 4-10 weeks. In some embodiments, the period of persisting signs and symptoms of pericarditis is 6-10 weeks. In some embodiments, the period of persisting signs and symptoms of pericarditis is 8-10 weeks. In some embodiments, the period of persisting signs and symptoms of pericarditis is 2-12 weeks. In some embodiments, the period of persisting signs and symptoms of pericarditis is 4-12 weeks. In some embodiments, the period of persisting signs and symptoms of pericarditis is 6-12 weeks. In some embodiments, the period of persisting signs and symptoms of pericarditis is 8-12 weeks. In some embodiments, the period of persisting signs and symptoms of pericarditis is 10-12 weeks.

In some embodiments, the period of persisting signs and symptoms of pericarditis is 2-8 weeks. In some embodiments, the period of persisting signs and symptoms of pericarditis is 2-6 weeks. In some embodiments, the period of persisting signs and symptoms of pericarditis is 2-4 weeks. In some embodiments, the period of persisting signs and symptoms of pericarditis is 4-6 weeks. In some embodiments, the period of persisting signs and symptoms of pericarditis is 4-8 weeks.

In some embodiments, the period of persisting signs and symptoms of pericarditis is greater than 1 week. In some embodiments, the period of persisting signs and symptoms of pericarditis is greater than 2 weeks. In some embodiments, the period of persisting is signs and symptoms of pericarditis is greater than 4 weeks. In some embodiments, the period of persisting signs and symptoms of pericarditis is greater than 6 weeks. In some embodiments, the period of persisting signs and symptoms of pericarditis is greater than 8 weeks. In some embodiments, the period of persisting signs and symptoms of pericarditis is greater than 10 weeks. In some embodiments, the period of persisting signs and symptoms of pericarditis is greater than 12 weeks.

In some embodiments, the period of persisting signs and symptoms of pericarditis is less than 6 weeks. In some embodiments, the period of persisting signs and symptoms of pericarditis is less than 5 weeks. In some embodiments, the period of persisting signs and symptoms of pericarditis is less than 4 weeks. In some embodiments, the period of persisting signs and symptoms of pericarditis is less than 3 weeks. In some embodiments, the period of persisting signs and symptoms of pericarditis is less than 2 weeks. In some embodiments, the period of persisting signs and symptoms of pericarditis is less than 1 week.

In some embodiments, the one or more symptoms of recurrent pericarditis are assessed by a Numerical Rating Scale (NRS) for assessment of pericarditis pain. In some embodiments, the one or more symptoms of recurrent pericarditis are assessed by an echocardiogram (ECG). In some embodiments, the one or more symptoms of pericarditis are assessed by cardiac magnetic resonance imaging (MRI). In some embodiments, the one or more symptoms of recurrent pericarditis are assessed by a Quality of Life Questionnaire, or a portion thereof (e.g., SF-36 Health Survey, or Patient Global Impression of Pericarditis Symptom Severity (PGIPS)).

In some embodiments, the one or more symptoms of recurrent pericarditis comprise fever. In some embodiments, the one or more symptoms of recurrent pericarditis comprise pericardial rub. In some embodiments, the one or more symptoms of recurrent pericarditis comprise an elevated level of C-reactive protein (CRP). In some embodiments, the one or more symptoms of recurrent pericarditis comprise chest pain. In some embodiments, the one or more symptoms of recurrent pericarditis comprise shortness of breath. In some embodiments, the one or more symptoms of recurrent pericarditis comprise heart palpitations. In some embodiments, the one or more symptoms of recurrent pericarditis comprise swelling. In some embodiments, the one or more symptoms of recurrent pericarditis is fatigue. In some embodiments, the one or more symptoms of recurrent pericarditis is the inability to exercise.

In some embodiments, the control is the patient prior to the administration of the anti-IL1R1 antibody. In some embodiments, the control is a comparable recipient without the administration of the anti-IL1R1 antibody. In some embodiments, the control is a comparable recipient administered a placebo. In some embodiments, the control is historical data.

In one aspect, the present invention provides, among other things, a method of treating recurrent pericarditis comprising administering to a patient, an anti-interleukin-1 receptor type-1 (IL1R1) antibody at a dose of greater than 10 mg.

In some embodiments, the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In some embodiments, the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of greater than 10 mg, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of greater than 10 mg, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of greater than 10 mg, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of greater than 10 mg, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In some embodiments, a HCDR1, a HCDR2, a HCDR3, LCDR1, a LCDR2, and a LCDR3 are determined by Kabat numbering system. In some embodiments, a HCDR1, a HCDR2, a HCDR3, LCDR1, a LCDR2, and a LCDR3 are determined by Chothia numbering system. In some embodiments, a HCDR1, a HCDR2, a HCDR3, LCDR1, a LCDR2, and a LCDR3 are determined by IMGT numbering system.

In some embodiments, the anti-IL1R1 antibody is administered no more frequently than once every week.

In one aspect, the present invention provides, among other things, a method of treating recurrent pericarditis comprising administering to a patient, an anti-interleukin-1 receptor type-1 (IL1R1) antibody at a therapeutically effective dose no more frequently than once every week.

In some embodiments, the patient has received a previous treatment. In some embodiments, the previous treatment is an IL1 antagonist, an IL1R1 antagonist, an IL1 trap, NSAIDs, colchicine, corticosteroid, or a combination thereof. In some embodiments, the previous treatment is an IL1 antagonist. In some embodiments, the previous treatment is an IL1R1 antagonist. In some embodiments, the previous treatment is an IL1 trap. In some embodiments, the previous treatment is NSAIDs. In some embodiments, the previous treatment is colchicine. In some embodiments, the previous treatment is corticosteroid. In some embodiments, the previous treatment is rilonacept or anakinra. In some embodiments, the previous treatment is rilonacept. In some embodiments, the previous treatment is anakinra.

In some embodiments, the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In some embodiments, the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a therapeutically effective dose no more frequently than once every week, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose no more frequently than once every week, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose no more frequently than once every week, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a therapeutically effective dose no more frequently than once every week, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In some embodiments, a HCDR1, a HCDR2, a HCDR3, LCDR1, a LCDR2, and a LCDR3 are determined by Kabat numbering system. In some embodiments, a HCDR1, a HCDR2, a HCDR3, LCDR1, a LCDR2, and a LCDR3 are determined by Chothia numbering system. In some embodiments, a HCDR1, a HCDR2, a HCDR3, LCDR1, a LCDR2, and a LCDR3 are determined by IMGT numbering system.

In some embodiments, the therapeutically effective dose produces a median Cₘₐₓ of at least 0.5 μg/mL. In some embodiments, the therapeutically effective dose produces a median Cₘₐₓ of at least 1.0 μg/mL. In some embodiments, the therapeutically effective dose produces a median Cmax of at least 5 μg/mL. In some embodiments, the therapeutically effective dose produces a median Cmax of at least 10 μg/mL. In some embodiments, the therapeutically effective dose produces a median Cmax of at least 30 μg/mL.

In some embodiments, the therapeutically effective dose maintains mean serum concentration greater than EC₉₀ for at least 7 days. In some embodiments, the therapeutically effective dose maintains mean serum concentration greater than EC₉₀ for at least 10 days. In some embodiments, the therapeutically effective dose maintains mean serum concentration greater than EC₉₀ for at least 15 days. In some embodiments, the therapeutically effective dose maintains mean serum concentration greater than EC₉₀ for at least 20 days. In some embodiments, the therapeutically effective dose maintains mean serum concentration greater than EC₉₀ for at least 25 days. In some embodiments, the therapeutically effective dose maintains mean serum concentration greater than EC₉₀ for at least 28 days. In some embodiments, the therapeutically effective dose maintains mean serum concentration greater than EC₉₀ for at least 56 days.

In some embodiments, the anti-IL1R1 antibody is administered at a dose of greater than 10 mg. In some embodiments, the anti-IL1R1 antibody is administered at a dose of greater than 30 mg. In some embodiments, the anti-IL1R1 antibody is administered at a dose of greater than 100 mg. In some embodiments, the anti-IL1R1 antibody is administered at a dose of greater than 300 mg.

In some embodiments, the anti-IL1R1 antibody is administered at a dose of at least 10 mg. In some embodiments, the anti-IL1R1 antibody is administered at a dose of at least 30 mg. In some embodiments, the anti-IL1R1 antibody is administered at a dose of at least 100 mg. In some embodiments, the anti-IL1R1 antibody is administered at a dose of at least 150 mg. In some embodiments, the anti-IL1R1 antibody is administered at a dose of at least 188 mg. In some embodiments, the anti-IL1R1 antibody is administered at a dose of at least 225 mg. In some embodiments, the anti-IL1R1 antibody is administered at a dose of at least 263 mg. In some embodiments, the anti-IL1R1 antibody is administered at a dose of at least 300 mg.

In some embodiments, the anti-IL1R1 antibody is administered at a dose of between 30 mg and 500 mg.

In some embodiments, the anti-IL1R1 antibody is administered at a dose of 10 mg. In some embodiments, the anti-IL1R1 antibody is administered at a dose of 30 mg. In some embodiments, the anti-IL1R1 antibody is administered at a dose of 50 mg. In some embodiments, the anti-IL1R1 antibody is administered at a dose of 100 mg. In some embodiments, the anti-IL1R1 antibody is administered at a dose of 150 mg. In some embodiments, the anti-IL1R1 antibody is administered at a dose of 188 mg. In some embodiments, the anti-IL1R1 antibody is administered at a dose of 225 mg. In some embodiments, the anti-IL1R1 antibody is administered at a dose of 250 mg. In some embodiments, the anti-IL1R1 antibody is administered at a dose of 263 mg. In some embodiments, the anti-IL1R1 antibody is administered at a dose of 300 mg. In some embodiments, the anti-IL1R1 antibody is administered at a dose of 325 mg. In some embodiments, the anti-IL1R1 antibody is administered at a dose of 350 mg. In some embodiments, the anti-IL1R1 antibody is administered at a dose of 500 mg.

In some embodiments, the anti-IL1R1 antibody is administered no more frequently than once every two weeks. In some embodiments, an anti-IL1R1 antibody is administered no more frequently than once every 3 weeks. In some embodiments, an anti-IL1R1 antibody is administered no more frequently than once every 4 weeks. In some embodiments, an anti-IL1R1 antibody is administered no more frequently than once every 5 weeks.

In some embodiments, the anti-IL1R1 antibody is administered at a dose of 100 mg once every two weeks. In some embodiments, the anti-IL1R1 antibody is administered at a dose of 100 mg once every four weeks. In some embodiments, the anti-IL1R1 antibody is administered at a dose of 150 mg once every two weeks. In some embodiments, the anti-IL1R1 antibody is administered at a dose of 150 mg once every four weeks. In some embodiments, the anti-IL1R1 antibody is administered at a dose of 188 mg once every two weeks. In some embodiments, the anti-IL1R1 antibody is administered at a dose of 188 mg once every four weeks. In some embodiments, the anti-IL1R1 antibody is administered at a dose of 225 mg once every two weeks. In some embodiments, the anti-IL1R1 antibody is administered at a dose of 225 mg once every four weeks. In some embodiments, the anti-IL1R1 antibody is administered at a dose of 250 mg once every two weeks. In some embodiments, the anti-IL1R1 antibody is administered at a dose of 250 mg once every four weeks. In some embodiments, the anti-IL1R1 antibody is administered at a dose of 263 mg once every two weeks. In some embodiments, the anti-IL1R1 antibody is administered at a dose of 263 mg once every four weeks. In some embodiments, the anti-IL1R1 antibody is administered at a dose of 275 mg once every two weeks. In some embodiments, the anti-IL1R1 antibody is administered at a dose of 275 mg once every four weeks. In some embodiments, the anti-IL1R1 antibody is administered at a dose of 300 mg once every two weeks. In some embodiments, the anti-IL1R1 antibody is administered at a dose of 300 mg once every four weeks.

In some embodiments, the anti-IL1R1 antibody is administered intravenously. In some embodiments, the anti-IL1R1 antibody is administered subcutaneously.

In some embodiments, the patient has at least 1 day with pericarditis pain measurement ≥4 on NRS prior to the treatment.

In some embodiments, the patient has C-reactive protein (CRP) value >0.5 mg/dL prior to the treatment. In some embodiments, the patient has CRP value ≥1.0 mg/dL prior to the treatment.

In some embodiments, the patient is an adult.

In some embodiments, the anti-IL1R1 antibody is administered once every four weeks.

In some embodiments, the patient has at least a second pericarditis recurrence prior to the treatment. In some embodiments, the patient has at least a third pericarditis recurrence prior to the treatment. In some embodiments, the patient has a period of persisting pericarditis signs and symptoms despite initial improvement.

In some embodiments, the patient is receiving concomitant NSAIDs, colchicine, corticosteroid, or combinations thereof. In some embodiments, the patient is receiving concomitant NSAIDs. In some embodiments, the patient is receiving concomitant colchicine. In some embodiments, the patient is receiving corticosteroid.

In some embodiments, the NSAID is ibuprofen.

In some embodiments, the administration of the anti-IL1R1 antibody results in a statistically-significant drop on a Numerical Rating Scale (NRS) for assessment of pericarditis pain.

In some embodiments, the administration of the anti-IL1R1 antibody results in a recurrence rate of 20% or less. In some embodiments, the administration of the anti-IL1R1 antibody results in a recurrence rate of 15% or less. In some embodiments, the administration of the anti-IL1R1 antibody results in a recurrence rate of 10% or less. In some embodiments, the administration of the anti-IL1R1 antibody results in a recurrence rate of 5% or less. In some embodiments, the administration of the anti-IL1R1 antibody results in a recurrence rate of 3% or less. In some embodiments, the administration of the anti-IL1R1 antibody results in a recurrence rate of 2% or less. In some embodiments, the administration of the anti-IL1R1 antibody results in a recurrence rate of 1% or less. In some embodiments, the administration of the anti-IL1R1 antibody results in a recurrence rate of 0%.

In some embodiments, the administration of the anti-IL1R1 antibody results in a median time-to-recurrence of greater than 10 weeks. In some embodiments, the administration of the anti-IL1R1 antibody results in a median time-to-recurrence of greater than 12 weeks. In some embodiments, the administration of the anti-IL1R1 antibody results in a median time-to-recurrence of greater than 16 weeks. In some embodiments, the administration of the anti-IL1R1 antibody results in a median time-to-recurrence of greater than 20 weeks. In some embodiments, the administration of the anti-IL1R1 antibody results in a median time-to-recurrence of greater than 24 weeks. In some embodiments, the administration of the anti-IL1R1 antibody results in a median time-to-recurrence of greater than 30 weeks. In some embodiments, the administration of the anti-IL1R1 antibody results in a median time-to-recurrence of greater than 40 weeks. In some embodiments, the administration of the anti-IL1R1 antibody results in a median time-to-recurrence of greater than 50 weeks.

In some embodiments, the median time-to-recurrence could not be determined due to a limited number of recurrence events.

In some embodiments, the limited number of recurrence events is less than 3. In some embodiments, the limited number of recurrence events is less than 1. In some embodiments, the limited number of recurrence events is substantially 0.

In some embodiments, the administration of the anti-IL1R1 antibody reduces a risk or recurrence by at least 60%. In some embodiments, the administration of the anti-IL1R1 antibody reduces a risk or recurrence by at least 70%. In some embodiments, the administration of the anti-IL1R1 antibody reduces a risk or recurrence by at least 80%. In some embodiments, the administration of the anti-IL1R1 antibody reduces a risk or recurrence by at least 90%. In some embodiments, the administration of the anti-IL1R1 antibody reduces a risk or recurrence by at least 95%. In some embodiments, the administration of the anti-IL1R1 antibody reduces a risk or recurrence by at least 96%. In some embodiments, the administration of the anti-IL1R1 antibody reduces a risk or recurrence by at least 97%. In some embodiments, the administration of the anti-IL1R1 antibody reduces a risk or recurrence by at least 98%. In some embodiments, the administration of the anti-IL1R1 antibody reduces a risk or recurrence by at least 99%. In some embodiments, the administration of the anti-IL1R1 antibody reduces a risk or recurrence by at least 100%.

In some embodiments, the administration of the anti-IL1R1 antibody as compared to the control results in a hazard ratio of less than 0.3. In some embodiments, the administration of the anti-IL1R1 antibody as compared to the control results in a hazard ratio of less than 0.2. In some embodiments, the administration of the anti-IL1R1 antibody as compared to the control results in a hazard ratio of less than 0.1. In some embodiments, the administration of the anti-IL1R1 antibody as compared to the control results in a hazard ratio of less than 0.05. In some embodiments, the administration of the anti-IL1R1 antibody as compared to the control results in a hazard ratio of less than 0.04. In some embodiments, the administration of the anti-IL1R1 antibody as compared to the control results in a hazard ratio of less than 0.03. In some embodiments, the administration of the anti-IL1R1 antibody as compared to the control results in a hazard ratio of less than 0.02. In some embodiments, the administration of the anti-IL1R1 antibody as compared to the control results in a hazard ratio of less than 0.01. In some embodiments, the administration of the anti-IL1R1 antibody as compared to the control results in a hazard ratio of less than 0.005.

In some embodiments, the administration of the anti-IL1R1 antibody results in NRS score of 6 or less. In some embodiments, the administration of the anti-IL1R1 antibody results in NRS score of 5 or less. In some embodiments, the administration of the anti-IL1R1 antibody results in NRS score of 4 or less. In some embodiments, the administration of the anti-IL1R1 antibody results in NRS score of 3 or less. In some embodiments, the administration of the anti-IL1R1 antibody results in NRS score of 2 or less. In some embodiments, the administration of the anti-IL1R1 antibody results in NRS score of 1 or less.

In some embodiments, the NRS score of 2 or less is achieved within 3 weeks from the first administration of the anti-IL1R1 antibody. In some embodiments, the NRS score of 2 or less is achieved within 2 weeks from the first administration of the anti-IL1R1 antibody. In some embodiments, the NRS score of 2 or less is achieved within 1 week from the first administration of the anti-IL1R1 antibody. In some embodiments, the NRS score of 2 or less is achieved within 5 days from the first administration of the anti-IL1R1 antibody. In some embodiments, the NRS score of 2 or less is achieved within 4 days from the first administration of the anti-IL1R1 antibody. In some embodiments, the NRS score of 2 or less is achieved within 3 days from the first administration of the anti-IL1R1 antibody. In some embodiments, the NRS score of 2 or less is achieved within 2 days from the first administration of the anti-IL1R1 antibody.

In some embodiments, the median time to achieve an NRS score of 2 or less is 7 days from the first administration of the anti-IL1R1 antibody. In some embodiments, the median time to achieve an NRS score of 2 or less is 6 days from the first administration of the anti-IL1R1 antibody. In some embodiments, the median time to achieve an NRS score of 2 or less is 5 days from the first administration of the anti-IL1R1 antibody. In some embodiments, the median time to achieve an NRS score of 2 or less is 4 days from the first administration of the anti-IL1R1 antibody. In some embodiments, the median time to achieve an NRS score of 2 or less is 3 days from the first administration of the anti-IL1R1 antibody. In some embodiments, the median time to achieve an NRS score of 2 or less is 2 days from the first administration of the anti-IL1R1 antibody.

In some embodiments, the NRS score is maintained at 2 or less for more than 1 week. In some embodiments, the NRS score is maintained at 2 or less for more than 2 weeks. In some embodiments, the NRS score is maintained at 2 or less for more than 3 weeks. In some embodiments, the NRS score is maintained at 2 or less for more than 1 month. In some embodiments, the NRS score is maintained at 2 or less for more than 2 months. In some embodiments, the NRS score is maintained at 2 or less for more than 3 months. In some embodiments, the NRS score is maintained at 2 or less for more than 4 months. In some embodiments, the NRS score is maintained at 2 or less for more than 5 months. In some embodiments, the NRS score is maintained at 2 or less for more than 6 months. In some embodiments, the NRS score is maintained at 2 or less for more than 8 months. In some embodiments, the NRS score is maintained at 2 or less for or more than 1 year.

In some embodiments, the NRS score of 1 or less is achieved within 3 weeks from the first administration of the anti-IL1R1 antibody. In some embodiments, the NRS score of 1 or less is achieved within 2 weeks from the first administration of the anti-IL1R1 antibody. In some embodiments, the NRS score of 1 or less is achieved within 1 week from the first administration of the anti-IL1R1 antibody. In some embodiments, the NRS score of 1 or less is achieved within 5 days from the first administration of the anti-IL1R1 antibody. In some embodiments, the NRS score of 1 or less is achieved within 4 days from the first administration of the anti-IL1R1 antibody. In some embodiments, the NRS score of 1 or less is achieved within 3 days from the first administration of the anti-IL1R1 antibody. In some embodiments, the NRS score of 1 or less is achieved within 2 days from the first administration of the anti-IL1R1 antibody.

In some embodiments, the median time to achieve an NRS score of 1 or less is 7 days from the first administration of the anti-IL1R1 antibody. In some embodiments, the median time to achieve an NRS score of 1 or less is 6 days from the first administration of the anti-IL1R1 antibody. In some embodiments, the median time to achieve an NRS score of 1 or less is 5 days from the first administration of the anti-IL1R1 antibody. In some embodiments, the median time to achieve an NRS score of 1 or less is 4 days from the first administration of the anti-IL1R1 antibody. In some embodiments, the median time to achieve an NRS score of 1 or less is 1 days from the first administration of the anti-IL1R1 antibody. In some embodiments, the median time to achieve an NRS score of 1 or less is 2 days from the first administration of the anti-IL1R1 antibody.

In some embodiments, the NRS score is maintained at 1 or less for more than 1 week. In some embodiments, the NRS score is maintained at 1 or less for more than 2 weeks. In some embodiments, the NRS score is maintained at 1 or less for more than 3 weeks. In some embodiments, the NRS score is maintained at 1 or less for more than 1 month. In some embodiments, the NRS score is maintained at 1 or less for more than 2 months. In some embodiments, the NRS score is maintained at 1 or less for more than 3 months. In some embodiments, the NRS score is maintained at 1 or less for more than 4 months. In some embodiments, the NRS score is maintained at 1 or less for more than 5 months. In some embodiments, the NRS score is maintained at 1 or less for more than 6 months. In some embodiments, the NRS score is maintained at 1 or less for more than 8 months. In some embodiments, the NRS score is maintained at 1 or less for or more than 1 year.

In some embodiments, administration of the anti-IL1R1 antibody results in a reduced CRP value of 2.0 mg/dL. In some embodiments, administration of the anti-IL1R1 antibody results in a reduced CRP value of ≤1.5 mg/dL. In some embodiments, administration of the anti-IL1R1 antibody results in a reduced CRP value of ≤1.0 mg/dL. In some embodiments, administration of the anti-IL1R1 antibody results in a reduced CRP value of ≤0.8 mg/dL. In some embodiments, administration of the anti-IL1R1 antibody results in a reduced CRP value of ≤0.6 mg/dL. In some embodiments, administration of the anti-IL1R1 antibody results in a reduced CRP value of ≤0.5 mg/dL. In some embodiments, administration of the anti-IL1R1 antibody results in a reduced CRP value of ≤0.4 mg/dL. In some embodiments, administration of the anti-IL1R1 antibody results in a reduced CRP value of ≤0.3 mg/dL. In some embodiments, administration of the anti-IL1R1 antibody results in a reduced CRP value of ≤0.2 mg/dL. In some embodiments, administration of the anti-IL1R1 antibody results in a reduced CRP value of ≤0.1 mg/dL.

In some embodiments, a reduced CRP value of ≤0.5 mg/dL is achieved within 3 weeks from the first administration of the anti-IL1R1 antibody. In some embodiments, a reduced CRP value of ≤0.5 mg/dL is achieved within 2 weeks from the first administration of the anti-IL1R1 antibody. In some embodiments, a reduced CRP value of ≤0.5 mg/dL is achieved within 1 week from the first administration of the anti-IL1R1 antibody. In some embodiments, a reduced CRP value of ≤0.5 mg/dL is achieved within 5 days from the first administration of the anti-IL1R1 antibody. In some embodiments, a reduced CRP value of ≤0.5 mg/dL is achieved within 4 days from the first administration of the anti-IL1R1 antibody. In some embodiments, a reduced CRP value of ≤0.5 mg/dL is achieved within 3 days from the first administration of the anti-IL1R1 antibody. In some embodiments, a reduced CRP value of ≤0.5 mg/dL is achieved within 2 days from the first administration of the anti-IL1R1 antibody.

In some embodiments, the median time to achieve a reduced CRP value of ≤0.5 mg/dL is within 3 weeks from the first administration of the anti-IL1R1 antibody. In some embodiments, the median time to achieve a reduced CRP value of ≤0.5 mg/dL is within 2 weeks from the first administration of the anti-IL1R1 antibody. In some embodiments, the median time to achieve a reduced CRP value of ≤0.5 mg/dL is within 1 week from the first administration of the anti-IL1R1 antibody. In some embodiments, the median time to achieve a reduced CRP value of ≤0.5 mg/dL is within 5 days from the first administration of the anti-IL1R1 antibody. In some embodiments, the median time to achieve a reduced CRP value of ≤0.5 mg/dL is within 4 days from the first administration of the anti-IL1R1 antibody. In some embodiments, the median time to achieve a reduced CRP value of ≤0.5 mg/dL is within 3 days from the first administration of the anti-IL1R1 antibody. In some embodiments, the median time to achieve a reduced CRP value of ≤0.5 mg/dL is within 2 days from the first administration of the anti-IL1R1 antibody.

In some embodiments, the CRP value ≤0.5 mg/dL is maintained for longer than 1 week. In some embodiments, the CRP value ≤0.5 mg/dL is maintained for longer than 2 weeks. In some embodiments, the CRP value ≤0.5 mg/dL is maintained for longer than 3 weeks. In some embodiments, the CRP value ≤0.5 mg/dL is maintained for longer than 1 month. In some embodiments, the CRP value ≤0.5 mg/dL is maintained for longer than 2 months. In some embodiments, the CRP value ≤0.5 mg/dL is maintained for longer than 3 months. In some embodiments, the CRP value ≤0.5 mg/dL is maintained for longer than 4 months. In some embodiments, the CRP value ≤0.5 mg/dL is maintained for longer than 5 months. In some embodiments, the CRP value ≤0.5 mg/dL is maintained for longer than 6 months. In some embodiments, the CRP value ≤0.5 mg/dL is maintained for longer than 8 months. In some embodiments, the CRP value ≤0.5 mg/dL is maintained for longer than 1 year.

In some embodiments, the median time to a treatment response is 5 days or less from the first administration of the anti-IL1R1 antibody.

In some embodiments, the % of subjects achieving a treatment response is 60%. In some embodiments, the % of subjects achieving a treatment response is 65%. In some embodiments, the % of subjects achieving a treatment response is 70%. In some embodiments, the % of subjects achieving a treatment response is 75%. In some embodiments, the % of subjects achieving a treatment response is 80%. In some embodiments, the % of subjects achieving a treatment response is 85%. In some embodiments, the % of subjects achieving a treatment response is 90%. In some embodiments, the % of subjects achieving a treatment response is 95%. In some embodiments, the % of subjects achieving a treatment response is 98%.

In some embodiments, the treatment response is NRS score of 2 or less and the CRP value ≤0.5 mg/dL. In some embodiments, the treatment response is NRS score of 1 or less and the CRP value ≤0.5 mg/dL. In some embodiments, the treatment response is NRS score of 2 or less and the CRP value ≤0.1 mg/dL. In some embodiments, the treatment response is NRS score of 1 or less and the CRP value ≤0.1 mg/dL. In some embodiments, the treatment response is NRS score of 1 or less and the CRP value ≤0.1 mg/dL.

In some embodiments, the median time to anti-IL1R1 antibody monotherapy is 4 weeks or less. In some embodiments, the median time to anti-IL1R1 antibody monotherapy is 6 weeks or less. In some embodiments, the median time to anti-IL1R1 antibody monotherapy is 7 weeks or less. In some embodiments, the median time to anti-IL1R1 antibody monotherapy is 7.5 weeks or less. In some embodiments, the median time to anti-IL1R1 antibody monotherapy is 8 weeks or less. In some embodiments, the median time to anti-IL1R1 antibody monotherapy is 10 weeks or less. In some embodiments, the median time to anti-IL1R1 antibody monotherapy is 12 weeks or less. In some embodiments, the median time to anti-IL1R1 antibody monotherapy is 14 weeks or less. In some embodiments, the median time to anti-IL1R1 antibody monotherapy is 16 weeks or less. In some embodiments, the median time to anti-IL1R1 antibody monotherapy is 18 weeks or less. In some embodiments, the median time to anti-IL1R1 antibody monotherapy is 20 weeks or less. In some embodiments, the median time to anti-IL1R1 antibody monotherapy is 22 weeks or less. In some embodiments, the median time to anti-IL1R1 antibody monotherapy is 24 weeks or less.

In some embodiments, the administration of the anti-IL1R1 antibody results in decreased pericardiac effusion compared to a control.

In some embodiments, the control is a baseline pericardiac effusion level measured in the patient prior to the treatment. In some embodiments, the control is a pericardiac effusion level measured in a patient with comparable disease status but administered a placebo. In some embodiments, the control is a reference value indicative of pericardiac effusion in a subject with comparable disease status without treatment.

In some embodiments, the administration of the anti-IL1R1 antibody results in absence of pericardiac effusion.

In some embodiments, the decreased or absence of pericardiac effusion is maintained for longer than 2 weeks. In some embodiments, the decreased or absence of pericardiac effusion is maintained for longer than 4 weeks. In some embodiments, the decreased or absence of pericardiac effusion is maintained for longer than 1 month. In some embodiments, the decreased or absence of pericardiac effusion is maintained for longer than 2 months. In some embodiments, the decreased or absence of pericardiac effusion is maintained for longer than 3 months. In some embodiments, the decreased or absence of pericardiac effusion is maintained for longer than 4 months. In some embodiments, the decreased or absence of pericardiac effusion is maintained for longer than 5 months. In some embodiments, the decreased or absence of pericardiac effusion is maintained for longer than 6 months. In some embodiments, the decreased or absence of pericardiac effusion is maintained for longer than 8 months. In some embodiments, the decreased or absence of pericardiac effusion is maintained for longer than 1 year.

In some embodiments, the administration of the anti-IL1R1 antibody results in a withdrawal, or a taper off, of a concurrent therapy or standard of care treatment in the patient. In some embodiments, the administration of the anti-IL1R1 antibody results in a withdrawal of a concurrent therapy or standard of care treatment in the patient. In some embodiments, the administration of the anti-IL1R1 antibody results in a taper off of a concurrent therapy or standard of care treatment in the patient.

In some embodiments, the concurrent therapy or standard of care treatment is NSAIDs. In some embodiments, the concurrent therapy or standard of care treatment is colchicine. In some embodiments, the concurrent therapy or standard of care treatment is corticosteroid.

In some embodiments, the administration of the anti-IL1R1 antibody does not result in an adverse event. In some embodiments, the adverse event is injection-site reaction. In some embodiments, the adverse event is upper respiratory tract infection. In some embodiments, the adverse event is headache. In some embodiments, the adverse event is nausea. In some embodiments, the adverse event is vomiting. In some embodiments, the adverse event is diarrhea. In some embodiments, the adverse event is sinusitis. In some embodiments, the adverse event is arthralgia. In some embodiments, the adverse event is flu-like symptoms. In some embodiments, the adverse event is abdominal pain. In some embodiments, the adverse event is pyrexia. In some embodiments, the adverse event is herpes. In some embodiments, the adverse event is transaminase elevation. In some embodiments, the adverse event is nasopharyngitis. In some embodiments, the adverse event is ischemic optic neuropathy. In some embodiments, the administration of the anti-IL1R1 antibody does not result in a dose-limiting adverse events (AE). In some embodiments, the administration of the anti-IL1R1 antibody does not result in a dose-related adverse events (AE). In some embodiments, the administration of the anti-IL1R1 antibody does not result in a dose-limiting or dose-related adverse events (AE).

In some embodiments, an anti-IL1R1 antibody comprises a VH that is at least 85% identical to SEQ ID NO: 7. In some embodiments, an anti-IL1R1 antibody comprises a VH that is at least 90% identical to SEQ ID NO: 7. In some embodiments, an anti-IL1R1 antibody comprises a VH that is at least 92% identical to SEQ ID NO: 7. In some embodiments, an anti-IL1R1 antibody comprises a VH that is at least 93% identical to SEQ ID NO: 7. In some embodiments, an anti-IL1R1 antibody comprises a VH that is at least 94% identical to SEQ ID NO: 7. In some embodiments, an anti-IL1R1 antibody comprises a VH that is at least 95% identical to SEQ ID NO: 7. In some embodiments, an anti-IL1R1 antibody comprises a VH that is at least 96% identical to SEQ ID NO: 7. In some embodiments, an anti-IL1R1 antibody comprises a VH that is at least 97% identical to SEQ ID NO: 7. In some embodiments, an anti-IL1R1 antibody comprises a VH that is at least 98% identical to SEQ ID NO: 7. In some embodiments, an anti-IL1R1 antibody comprises a VH that is at least 99% identical to SEQ ID NO: 7. In some embodiments, an anti-IL1R1 antibody comprises a VH of SEQ ID NO: 7.

In some embodiments, an anti-IL1R1 antibody comprises a VL that is at least 85% identical to SEQ ID NO: 8. In some embodiments, an anti-IL1R1 antibody comprises a VL that is at least 90% identical to SEQ ID NO: 8. In some embodiments, an anti-IL1R1 antibody comprises a VL that is at least 92% identical to SEQ ID NO: 8. In some embodiments, an anti-IL1R1 antibody comprises a VL that is at least 93% identical to SEQ ID NO: 8. In some embodiments, an anti-IL1R1 antibody comprises a VL that is at least 94% identical to SEQ ID NO: 8. In some embodiments, an anti-IL1R1 antibody comprises a VL that is at least 95% identical to SEQ ID NO: 8. In some embodiments, an anti-IL1R1 antibody comprises a VL that is at least 96% identical to SEQ ID NO: 8. In some embodiments, an anti-IL1R1 antibody comprises a VL that is at least 97% identical to SEQ ID NO: 8. In some embodiments, an anti-IL1R1 antibody comprises a VL that is at least 98% identical to SEQ ID NO: 8. In some embodiments, an anti-IL1R1 antibody comprises a VL that is at least 99% identical to SEQ ID NO: 8. In some embodiments, an anti-IL1R1 antibody comprises a VL of SEQ ID NO: 8.

In some embodiments, the anti-IL1R1 comprises a VH of SEQ ID NO: 7 and a VL of SEQ ID NO: 8.

In some embodiments, the anti-IL1R1 antibody comprises an Fc region derived from IgG1. In some embodiments, the anti-IL1R1 antibody comprises an Fc region derived from IgG2. In some embodiments, the anti-IL1R1 antibody comprises an Fc region derived from IgG3. In some embodiments, the anti-IL1R1 antibody comprises an Fc region derived from IgG4.

In some embodiments, an Fc region comprises an amino acid sequence that is at least 85% identical to SEQ ID NO: 9. In some embodiments an Fc region comprises an amino acid sequence that is at least 90% identical to SEQ ID NO: 9. In some embodiments an Fc region comprises an amino acid sequence that is at least 92% identical to SEQ ID NO: 9. In some embodiments an Fc region comprises an amino acid sequence that is at least 93% identical to SEQ ID NO: 9. In some embodiments an Fc region comprises an amino acid sequence that is at least 94% identical to SEQ ID NO: 9. In some embodiments an Fc region comprises an amino acid sequence that is at least 95% identical to SEQ ID NO: 9. In some embodiments an Fc region comprises an amino acid sequence that is at least 96% identical to SEQ ID NO: 9. In some embodiments an Fc region comprises an amino acid sequence that is at least 97% identical to SEQ ID NO: 9. In some embodiments, an Fc region comprises an amino acid sequence that is at least 98% identical to SEQ ID NO: 9. In some embodiments an Fc region comprises an amino acid sequence that is at least 99% identical to SEQ ID NO: 9. In some embodiments, the Fc region comprises an amino acid sequence of SEQ ID NO: 9.

In some embodiments, an anti-IL1R1 antibody comprises a heavy chain that is at least 85% identical to SEQ ID NO: 10. In some embodiments, an anti-IL1R1 antibody comprises a heavy chain that is at least 90% identical to SEQ ID NO:10. In some embodiments, an anti-IL1R1 antibody comprises a heavy chain that is at least 92% identical to SEQ ID NO:10. In some embodiments, an anti-IL1R1 antibody comprises a heavy chain that is at least 93% identical to SEQ ID NO:10. In some embodiments, an anti-IL1R1 antibody comprises a heavy chain that is at least 94% identical to SEQ ID NO:10. In some embodiments, an anti-IL1R1 antibody comprises a heavy chain that is at least 95% identical to SEQ ID NO:10. In some embodiments, an anti-IL1R1 antibody comprises a heavy chain that is at least 96% identical to SEQ ID NO:10. In some embodiments, an anti-IL1R1 antibody comprises a heavy chain that is at least 97% identical to SEQ ID NO:10. In some embodiments, an anti-IL1R1 antibody comprises a heavy chain that is at least 98% identical to SEQ ID NO:10. In some embodiments, an anti-IL1R1 antibody comprises a heavy chain that is at least 99% identical to SEQ ID NO:10. In some embodiments, an anti-IL1R1 antibody comprises a heavy chain of SEQ ID NO:10.

In some embodiments, an anti-IL1R1 antibody comprises a light chain that is at least 85% identical to SEQ ID NO: 11. In some embodiments, an anti-IL1R1 antibody comprises a light chain that is at least 90% identical to SEQ ID NO: 11. In some embodiments, an anti-IL1R1 antibody comprises a light chain that is at least 92% identical to SEQ ID NO:11. In some embodiments, an anti-IL1R1 antibody comprises a light chain that is at least 93% identical to SEQ ID NO: 11. In some embodiments, an anti-IL1R1 antibody comprises a light chain that is at least 94% identical to SEQ ID NO: 11. In some embodiments, an anti-IL1R1 antibody comprises a light chain that is at least 95% identical to SEQ ID NO: 11. In some embodiments, an anti-IL1R1 antibody comprises a light chain that is at least 96% identical to SEQ ID NO: 11. In some embodiments, an anti-IL1R1 antibody comprises a light chain that is at least 97% identical to SEQ ID NO: 11. In some embodiments, an anti-IL1R1 antibody comprises a light chain that is at least 98% identical to SEQ ID NO: 11. In some embodiments, an anti-IL1R1 antibody comprises a light chain that is at least 99% identical to SEQ ID NO: 11. In some embodiments, an anti-IL1R1 antibody comprises a light chain of SEQ ID NO: 11.

In some embodiments, the anti-IL1R1 antibody comprises a heavy chain of SEQ ID NO: 10 and a light chain of SEQ ID NO: 11.

### BRIEF DESCRIPTION OF FIGURES

The drawings are for illustration purposes only not for limitation.

FIG. 1A is an exemplary graph showing observed concentrations of KPL-387 over time after a single administration of 3 mg, 10 mg, 30 mg, 100 mg, 300 mg, or 600 mg of KPL-387. FIG. 1B is an exemplary graph showing observed and predicted concentrations of KPL-387 over time after a single administration of 300 mg of KPL-387. FIG. 1C and FIG. 1D are exemplary box and whisker plots of observed PK parameters and NHP Pop PK-predicted values in the 300 mg cohort up to 70 days.

FIG. 2 is an exemplary graph showing observed concentrations of KPL-387 over time in healthy human participants after subcutaneous administration of 100 mg once every four weeks, 300 mg once every four weeks, and 300 mg once every two weeks.

FIG. 3 is an exemplary study schematic for Phase 2 (Part A) and Long-Term Extension (LTE; Part C).

FIG. 4 is an exemplary study schematic for Phase 3 (Part B) and Long-Term Extension (LTE; Part C).

FIG. 5 is an exemplary schematic for evaluation and clinical management of suspected pericarditis recurrence.

FIG. 6 is an exemplary graph showing the NRS scores and CRP levels for Subject X after administration of KPL-387.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Any numerical values used in this application are meant to cover any variations within the standard deviation or normal fluctuations appreciated by one of ordinary skill in the relevant art.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxyl- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes increasing levels of relevant protein or its activity that is deficient in relevant disease tissues. In some embodiments, amelioration includes decreasing levels of relevant protein or its activity that is pathologically elevated in relevant disease tissues.

Delivery: As used herein, the term “delivery” encompasses both local and systemic delivery.

Equivalent dosing regimen: As used herein, the term “equivalent dosing regimen” or other grammatical equivalents refer to an alternative dosing regimen that delivers a substantially equivalent amount of a therapeutic agent (e.g., an antibody, a protein or small molecular compound drug) over a comparable dosing period or otherwise results in similar pharmacodynamic (PD) and/or pharmacokinetic (PK) response in vivo. As non-limiting examples, an equivalent dosing regimen of 100 mg once every 2 weeks can be 50 mg once a week, 200 mg once every four weeks, or 600 mg once every six weeks. In some embodiments, an equivalent dosing regimen may achieve a similar area under the curve (AUC), C_{average}, Cₘᵢₙ, EC₉₀ and/or Emax over a comparable dosing period.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein, e.g., a subject who is administered a placebo. A “control subject” is a subject with the same disease as the subject being treated, but who received no treatment, or who received placebo.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLAS TN for nucleotide sequences and BLASTP, gapped BLAST, and PSI-BLAST for amino acid sequences. Exemplary such programs are described in Altschul, et al., Basic local alignment search tool, J Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Patient: As used herein, the term “patient” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Recurrence: As used herein, the term “recurrence” is defined as the recurrence of typical pericarditis pain associated with supportive objective evidence of pericarditis. Recurrence is often used interchangeably with “flare” and “relapse”. A pericarditis recurrence is usually indicated by any one or more of the following: an increase in the CRP level of ≥1 mg/dl in peripheral blood; or an increase in pain, determined in an NRS scale of ≥4; or occurrence of pericardial effusion; a pericardial rub; or fever or any other symptomatic indication of the pericardial disease.

Initial Improvement: As used herein, the term “initial improvement” as used in connection with recurrent pericarditis patients refers to improvement of one or more signs or symptoms associated with an episode of acute pericarditis (also referred to as a flare) resulting from a treatment, including but not limited to reduction or improvement in one of more of: a) pericarditis pain; b) CRP; c) pericardial rub; d) widespread ST segment elevation or PR segment depression according to ECG findings; e) pericardial effusion; f) pericardial inflammation; g) edema; h) evidence of pericarditis by late gadolinium enhancement; i) evidence of constriction; and j) fever; chest pain; shortness of breath; and/or heart palpitations. In some embodiments, pericarditis pain is measured by NRS.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Target tissues: As used herein, the term “target tissues” refers to any tissue that is affected by a disease or disorder to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce the incidence or recurrence, or the risk thereof, of one or more signs, symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, a convenient, more tolerable and safer method for treating recurrent pericarditis (RP) based on an anti-IL1R1 antibody using dosing regimens described herein.

### Pericarditis and Recurrent Pericarditis

Pericarditis is swelling and irritation of the pericardium, the thin saclike membrane surrounding the heart. Pericarditis often causes chest pain and sometimes other symptoms. The sharp chest pain associated with pericarditis occurs when the irritated layers of the pericardium rub against each other. Signs and symptoms of pericarditis may include some or all of the following: sharp, piercing chest pain over the center or left side of the chest, which is generally more intense when breathing in or reclining; shortness of breath when reclining; heart palpitations; low-grade fever; an overall sense of weakness, fatigue or feeling sick; cough; and abdominal or leg swelling.

Pericarditis accounts for 5% of emergency department visits for chest pain in the absence of myocardial infarction (Khandaker et al, Mayo Clin Proc. 2010; 85:572-593). In 80% of cases in developed countries, the cause of pericarditis is either post viral or “idiopathic,” in that it cannot be attributed to a specific condition (Imazio et al, Circulation. 2010; 121:916-928; Zayas et al, Am J Cardiol. 1995; 75:378-382). Diagnosis is based on the presence of typical chest pain (improved by sitting up and leaning forward) along with fever, pericardial friction rub, electrocardiographic (ECG) changes, pericardial effusion, or elevated markers of inflammation (white blood cell [WBC] count, C-reactive protein [CRP], or erythrocyte sedimentation rate [ESR]) (Imazio, Revista Espanola de Cardiologia. 2014; 67(5):345-348). The European Society of Cardiology (ESC) Guidelines for the Diagnosis and Management of Pericardial Diseases define a pericarditis episode as the presence of at least 2 of the 4 following criteria: pericarditic chest pain, pericardial rubs, new widespread ST-elevation or PR depression on ECG, and pericardial effusion (new or worsening). Elevations of markers of inflammation (i.e., CRP, ESR, and WBD) or evidence of pericardial inflammation by an imaging technique (e.g., magnetic resonance imaging [MRI]) are used as supportive findings (Adler et al, Eur Heart J. 2015 Nov. 7; 36(42):2921-64.

The cause of pericarditis is often hard to determine. In most cases, doctors are either unable to determine a cause (idiopathic) or suspect a viral infection. Although the underlying pathogenesis of idiopathic recurrent pericarditis (RIP) (sometimes used interchangeably with recurrent idiopathic pericarditis) remains unclear, immune-mediated mechanisms are believed to play a key role in the pathogenesis (Imazio et al, 2005; 96(5):736-739). A growing body of evidence suggests that these immune responses consist of both pathogenic autoimmune and auto-inflammatory processes (Cantarini et al, Autoimmunity Reviews 2015; 14:90-97; Doria et al, Autoimmunity Reviews 2012; 12:22-30). The presence of pro-inflammatory cytokines in the pericardial fluid of RIP patients lends direct support to both an autoimmune and/or auto-inflammatory etiopathogenesis (Pankuwait et al, 2000).

Recurrent pericarditis (RP) is a rare autoinflammatory disease which affects 20% to 30% of patients after an incident acute pericarditis event (Imazio 2014) and can be debilitating due to pain and limitations in physical function. Typically, RP is characterized by an initial acute pericarditis event followed by one or more subsequent acute recurrences after a symptom-free interval, e.g. of 4-6 weeks (Adler 2015). In some cases, however, RP patients can have persisting signs and symptoms of pericarditis despite initial improvement of the initial acute pericarditis event without a symptom-free interval. In some embodiments, a recurrent pericarditis is an incessant pericarditis.

In some embodiments, the initial improvement is an improvement of one or more signs and symptoms associated with an episode of acute pericarditis. In some embodiments, the initial improvement comprises a reduction or improvement in one or more of.
a) pericarditis pain;
b) CRP;
c) pericardial rub;
d) widespread ST segment elevation or PR segment depression according to ECG findings;
e) pericardial effusion;
f) pericardial inflammation;
g) edema;
h) evidence of pericarditis by late gadolinium enhancement;
i) evidence of constriction; and
j) fever, chest pain, shortness of breath, and/or heart palpitations.

Typically, “persisting signs and symptoms of pericarditis,” “persistent pericarditis signs and symptoms” or its grammatical equivalents include one or more of (1) pericarditis pain; (2) an elevated CRP level; (3) pericardial rub; (4) widespread ST segment elevation or PR segment depression according to ECG findings; (5) pericardial effusion; (6) pericardial inflammation; (7) edema; (8) evidence of pericarditis by late gadolinium enhancement; (9) evidence of constriction; and (10) fever, chest pain, shortness of breath, and/or heart palpitations.

Pericarditis recurrences increase rates of emergency room admissions and hospitalizations. In the United States, 5% of patients presenting to the emergency room with non-ischemic chest pain are diagnosed with pericarditis (Agarwal 2015).

Potentially life-threatening complications of recurrent pericarditis include cardiac tamponade and constrictive pericarditis. Recurrent pericarditis may also result in the need for invasive surgery (i.e., pericardiectomy) in patients who are refractive to conventional treatments. The estimated prevalence of RP in the United States is approximately 40,000 patients, of whom approximately 14,000 experience multiple recurrences, may be refractory or intolerant to current therapeutic options, require long-term administration of corticosteroids to control their disease, and have persistent underlying disease (Imazio M, Brucato A, Trinchero R, Shabetai R, Spodick D, Adler Y. Corticosteroid therapy for pericarditis: a double-edged sword. Nature Clinical Practice, 2008 March; 5:3:118-119.; Imazio M, Spodick D H, Brucato A, Trinchero R, Adler Y. Controversial issues in the management of pericardial diseases. Circulation. 2010; 121:916-928.; Khandaker M H, Espinosa R E, Nishimura R A, Sinak L J, Hayes S N, Melduni R M, Oh J K. Pericardial disease: diagnosis and management. Mayo Clin Proc. 2010; 85:572-593.; Lazaros G, Imazio M, Brucato A, et al. Anakinra: an emerging option for refractory idiopathic recurrent pericarditis: a systematic review of published evidence. J Cardiovasc Med. 2016; 17:256-262.; Brucato A, Brambilla G, Moreo A, Alberti A, Munforti C, Ghirardello A, et al. Long-term outcomes in difficult-to-treat patients with recurrent pericarditis. Am J Cardiol. 2006; 98:267-271.). Recurrent pericarditis is a serious, chronic, flaring disease which can last for years. In patients with two or more recurrences, the median duration of disease is 3 years, with one-third of patients still suffering at 5 years and one-quarter still suffering at 8 years.

Therapeutic options for the management of pericarditis episodes include nonsteroidal anti-inflammatory drugs (NSAIDs), colchicine, and/or corticosteroids, in sequential order (Lilly S L. Treatment of Acute and Recurrent Idiopathic Pericarditis. Circulation. 2013; 127:1723-1726). Aspirin and other NSAIDs are the first-line approach. Because high doses of NSAIDs are often required, consideration must be given to gastric protection therapy, as potential risks of therapy include stomach ulcers and gastrointestinal (GI) bleeding as well as renal and cardiovascular toxicity. Colchicine is commonly used with NSAIDs to hasten the response to NSAIDs and reduce the risk of subsequent pericarditis recurrences. However, its use may be associated with the risks of fatal overdosing, significant drug-drug interactions, and neuromuscular toxicity. In addition, appropriately 10% to 15% of patients experience significant GI side effects with colchicine, including GI intolerance or severe diarrhea, requiring treatment discontinuation (Imazio M, Adler Y, Charron P. Recurrent pericarditis: Modern approach in 2016. Curr Cardiol Rep. 2016; 18(6):50).

Patients with recurrent pericarditis who are refractory or intolerant to current therapeutic management options or who require long-term administration of corticosteroids and/or colchicine to control their disease can be challenging to manage. Some refractory patients, as a last resort, are referred for the surgical procedure of percardiectomy, with variable outcomes. Multiple immunosuppressive medications have been tried on an exploratory basis without consistent benefit (Brucato A, Imazio M, Gattorno M, Lazaros G, Maestroni S, Carraro M, Finetti M et al. Effect of anakinra on recurrent pericarditis among patients with colchicine resistance and corticosteroid dependence: The AIRTRIP Randomized Clinical Trial. JAMA. 2016 Nov. 8; 316(18):1906-1912).

Interleukin-1 (IL-1) is a key cytokine that drives the pathophysiology of many inflammatory processes. It is implicated as a causative factor in various inflammatory human diseases. Although the pathogenic mechanism of auto-inflammatory disease is not completely understood, there is a growing body of evidence that IL-1 may be a primary driver of the symptomology and that targeting this cytokine may provide important benefits (Hoffman & Patel, Arthritis and Rheum. 2004 February; 50(2): 345-349). In fact, a study of once-daily anakinra (KINERET®), a recombinant form of the human IL-1 receptor antagonist (IL-1RA), showed promising effects in RIP patients when colchicine failed and corticosteroid dependence (or intolerance) developed, with C-reactive protein (CRP) normalization within a mean of 7.1 days in 21 consecutively treated patients (Brucato et al, JAMA. 2016 Nov. 8; 316(18):1906-1912; Lazaros et al, J Cardiovasc Med 2016; 17(4):256-62). However, anakinra is a once-daily injection and is known to cause injection site reaction among other adverse events.

### Patients

The method described herein is suitable for treating recurrent pericarditis patients that have (1) a second recurrence with an initial acute pericarditis episode having been followed by a symptom-free interval; or (2) at least one episode of acute pericarditis followed by a period of persisting signs and symptoms of pericarditis despite initial improvement. The patients in group (2) do not experience a symptom-free interval.

In some embodiments, prior to the treatment, a patient presents with signs and symptoms of acute pericarditis (Qualifying Pericarditis Episode) despite treatment with standard therapies. In some embodiments, the patient has received a previous treatment for recurrent pericarditis. Exemplary treatments include, without limitation, IL1 antagonists, IL1R1 antagonists, IL1 traps, NSAIDs, colchicine, corticosteroids, or a combination thereof. In some embodiments, the patient received a previous treatment of rilonacept or anakinra.

In some embodiments, a patient has a history, or current clinical diagnosis, of pericarditis that is consistent with the 2015 ESC Guidelines for the Diagnosis and Management of Pericardial Diseases (Adler 2015), fulfilling at least 2 of the following 4 criteria: (1) Pericarditic chest pain; (2) Pericardial rub; (3) New widespread ST segment elevation or PR segment depression according to ECG findings; and (4) Pericardial effusion (new or worsening).

In some embodiments, a patient has a qualifying pericarditis episode prior to the treatment. In some embodiments, a qualifying pericarditis episode is at least 1 day with pericarditis pain measurement ≥4 on the 11-point NRS and at least 1 CRP level ≥1 mg/dL from a sample drawn within 7 days before or after the qualifying NRS.

In some embodiments, a patient has at least 1 day with pericarditis pain measurement ≥4 on NRS prior to the treatment.

In some embodiments, a patient has a C-reactive protein (CRP) value >0.5 mg/dL prior to the treatment.

In some embodiments, a patient has a CRP value ≥1.0 mg/dL prior to the treatment.

In some embodiments, prior to the treatment, a patient has an initial acute pericarditis episode followed by one or more pericarditis recurrences after an interval. In some embodiments, the patient has at least a first pericarditis recurrence (i.e., at least the second pericarditis episode) after an initial acute pericarditis episode that was followed by a symptom-free interval. In some embodiments, the patient has at least a second pericarditis recurrence (i.e., at least the third pericarditis episode) after an initial acute pericarditis episode that was followed by a symptom-free interval. In some embodiments, the patient has at least a third pericarditis recurrence (i.e., at least the fourth pericarditis episode) after an initial acute pericarditis episode that was followed by a symptom-free interval. In some embodiments, prior to the treatment, a patient has an initial acute pericarditis episode followed by one or more flares without a symptom-free interval. In some embodiments, prior to the treatment, a patient has persisting signs and symptoms of pericarditis despite initial improvement, without a symptom-free interval.

In some embodiments, prior to the treatment, the patient has at least one episode of acute pericarditis with pericarditis pain NRS ≥4 and CRP ≥1.0 mg/dL followed by a period of persisting signs and symptoms of pericarditis signs and symptoms despite initial improvement.

In some embodiments, prior to the treatment, the patient has persistent pericarditis signs and symptoms despite initial improvement without a symptom free interval.

In some embodiments, the patient has at least one episode of acute pericarditis with pericarditis pain NRS ≥4 and CRP ≥1.0 mg/dL followed by a period of persisting signs and symptoms of pericarditis despite initial improvement. As used herein, a patient with persisting signs and symptoms of pericarditis typically has one or more of (1) pericarditis pain; (2) an elevated CRP level; (3) pericardial rub; (4) widespread ST segment elevation or PR segment depression according to ECG findings; (5) pericardial effusion; (6) pericardial inflammation; (7) edema; (8) evidence of pericarditis by late gadolinium enhancement; (8) evidence of constriction; and (9) fever, chest pain, shortness of breath, and/or heart palpitations.

In some embodiments, the patient with persisting pericarditis signs and symptoms of pericarditis has pericarditis pain. In some embodiments, the patient with persisting pericarditis signs and symptoms of pericarditis has pericarditis pain as measured by a NRS score ≥1. In some embodiments, the NRS score is ≥2. In some embodiments, the NRS score is ≥3. In some embodiments, the NRS score is ≥4.

In some embodiments, the patient with persisting signs and symptoms of pericarditis has an elevated CRP level. In some embodiments, the elevated CRP level is ≥1 mg/dL. In some embodiments, the elevated CRP level is ≥0.5 mg/dL. In some embodiments, the elevated CRP level is ≥0.6 mg/dL. In some embodiments, the elevated CRP level is ≥0.7 mg/dL. In some embodiments, the elevated CRP level is ≥0.8 mg/dL. In some embodiments, the elevated CRP level is ≥0.9 mg/dL.

In some embodiments, the patient with persisting signs and symptoms of pericarditis does not have a combination of pericarditis pain as measured by a NRS score ≥4 and an elevated CRP level ≥1 mg/dL.

In some embodiments, the patient with persisting signs and symptoms of pericarditis has pericardial rub. In some embodiments, the patient with persisting signs and symptoms of pericarditis has widespread ST segment elevation or PR segment depression according to ECG findings. In some embodiments, the patient with persisting signs and symptoms of pericarditis has pericardial effusion. In some embodiments, the patient with persisting signs and symptoms of pericarditis has pericardial inflammation. In some embodiments, the patient with persisting signs and symptoms of pericarditis has edema. In some embodiments, the patient with persisting evidence of pericarditis has evidence of pericarditis by late gadolinium enhancement according to cardiac MRI.

In some embodiments, the patient with persisting signs and symptoms of pericarditis has evidence of constriction. In some embodiments, the evidence of constriction is according to ECG findings.

In some embodiments, the patient with persisting pericarditis signs and symptoms has fever, chest pain, shortness of breath, heart palpitations or a combination thereof. In some embodiments, the patient with persisting signs and symptoms of pericarditis has fever. In some embodiments, the patient with persisting signs and symptoms of pericarditis has chest pain. In some embodiments, the patient with persisting signs and symptoms of pericarditis has shortness of breath. In some embodiments, the patient with persisting signs and symptoms of pericarditis has heart palpitations.

In some embodiments of the methods described herein, the patient is a pediatric patient. In some embodiments, the pediatric patient is ≥12 and <18 years old. In some embodiments, the pediatric patient is ≥6 and <18 years old. In some embodiments, the pediatric patient is ≥6 and ≤12 years old. In some embodiments, the pediatric patient is <6 years old. In some embodiments, the pediatric patient is <18 years old.

### Anti-IL1R1 Antibodies

The anti-interleukin-1 receptor type-1 (IL1R1) antibodies provide an advantageous mechanism of action for antagonizing both IL-1α and IL-1β signaling and are beneficial for treating recurrent pericarditis according to methods described in the present invention.

An anti-IL1R1 antibody described herein is a promising candidate for the treatment of patients with RP based on 1) the IL-1-antagonistic properties and in vivo pharmacokinetics (PK), which may enable monthly dosing in humans, 2) the established and favorable safety profile, and 3) the more convenient dosing schedule and easier dose administration.

In one aspect, the present invention provides, among other things, a method of treating recurrent pericarditis comprising administering to a patient, an anti-IL1R1 antibody at a therapeutically effective dose sufficient to improve, stabilize, prevent or reduce one or more symptoms of recurrent pericarditis relative to a control.

### Antibody

A traditional antibody, also known as an immunoglobulin, is a Y-shaped structuring consisting of four polypeptides, two heavy chains and two light chains. The light chain consists of one variable region (VL) and one constant region (CL). The heavy chain consists of one variable region (VH) and at least three constant regions (CH1, CH2, CH3). The two light and two heavy chains are joined by disulfide bonds in the Y-shaped structure, with the light chains bracketing the heavy chains at the mouth of the Y-shape and continuing through the variable region. The variable regions determine the binding specificity of the antibody. Each variable region contains three hypervariable regions known as complementarity determining regions (CDRs) flanked by four relatively conserved regions known as framework regions (FRs). The three CDRs, referred to as CDR1, CDR2, and CDR3, contribute to the antibody binding specificity. Naturally occurring antibodies have often been used as starting material for engineered antibodies, such as chimeric antibodies and humanized antibodies. As used herein, an antibody described herein include antigen-binding fragments, including but not limited to Fab, Fab′, (Fab′)2, Fv, single chain antibodies (e.g., scFv), single-domain antibodies (e.g., sdAbs, VHH, nanobodies), Fd (fragment antigen-binding domain), dAb (domain antibody), biFab (bivalent Fab), minibodies, diabodies, and any other antigen-binding fragments known in the art. Examples of antibodies that have been modified or engineered include chimeric antibodies, humanized antibodies, and multispecific antibodies (e.g., bispecific antibodies).

Different antibody numbering schemes have been developed for accurate identification and subsequent comparison of variable regions of different antibodies. The most commonly used numbering schemes include Kabat, Chothia, and IMGT.

The Kabat numbering scheme was one of the first systems used to identify CDRs and is based on the analysis of antibody sequences, particularly the immunoglobulin variable (V) regions. In this scheme, CDRs are defined based on sequence variability, with CDRs being those segments of the variable regions that show the highest variability across different antibodies. Typically, in the Kabat numbering system, LCDR1 spans residues 24-34, LCDR2 spans residues 50-56, LCDR3 spans residues 89-97, HCDR1 spans residues 31-35, HCDR2 spans residues 50-65, and HCDR3 spans residues 95-102.

The Chothia numbering system is based on a structural analysis of antibody sequences and structures. It assigns residue numbers based on the positions of the amino acids in the context of the three-dimensional structure of the antibody. CDRs in Chothia numbering system scheme are defined based on structural features, primarily the loop regions that are responsible for binding to antigens. Typically, in the Chothia numbering system, LCDR1 spans residues 24-34, LCDR2 spans residues 50-56, LCDR3 spans residues 89-97, HCDR1 spans residues 27-35, HCDR2 spans residues 52-56, and HCDR3 spans residues 95-102.

The IMGT numbering scheme is based on an extensive analysis of the immunoglobulin sequences in the IMGT database, which includes human and other species' sequences. This scheme is a more recent development and is the most widely used in genomics and bioinformatics tools. It defines CDRs based on the concept of framework regions and focuses on the sequence alignment of antibodies. Typically, in the IMGT numbering system, LCDR1 spans residues 27-38, LCDR2 spans residues 56-65, LCDR3 spans residues 105-117, HCDR1 spans residues 27-38, HCDR2 spans residues 56-65, and HCDR3 spans residues 105-117.

Table 1 below shows a summary of typical CDR residue numbers by different numbering schemes.

TABLE 1
Amino Acid Residues of CDRs by Different Numbering Schemes

| Scheme | LCDR1 | LCDR2 | LCDR3 | HCDR1 | HCDR2 | HCDR3 |
|---|---|---|---|---|---|---|
| Kabat | 24-34 | 50-56 | 89-97 | 31-35 | 50-65 | 95-102 |
| Chothia | 24-34 | 50-56 | 89-97 | 27-35 | 52-56 | 95-102 |
| IMGT | 27-38 | 56-65 | 105-117 | 27-38 | 56-65 | 105-117 |

CDR sequences of an antibody can be determined by a known method in the art, for example, at website: aligncdr.labshare.cn/aligncdr/abrsa.php.

In some embodiments, a LCDR1, a LCDR2, and a LCDR3 of an antibody comprise amino acid residues 24-34, 50-56, and 89-97, respectively, of a variable light chain (VL). In some embodiments, a HCDR1, a HCDR2, and a HCDR3 of an antibody comprise amino acid residues 31-35, 50-65, and 95-102, respectively, of a variable heavy chain (VH).

In some embodiments, a LCDR1, a LCDR2, and a LCDR3 of an antibody comprise amino acid residues 24-34, 50-56, and 89-97, respectively, of a variable light chain (VL). In some embodiments, a HCDR1, a HCDR2, and a HCDR3 of an antibody comprise amino acid residues 27-35, 52-56, and 95-102, respectively, of a variable heavy chain (VH).

In some embodiments, a LCDR1, a LCDR2, and a LCDR3 of an antibody comprise amino acid residues 27-38, 56-65, and 105-117, respectively, of a variable light chain (VL). In some embodiments, a HCDR1, a HCDR2, and a HCDR3 of an antibody comprise amino acid residues 27-38, 56-65, and 105-117, respectively, of a variable heavy chain (VH).

### Exemplary Anti-IL1R1 Antibody Sequences

In some embodiments, an anti-IL1R1 antibody comprises a variable heavy chain (VH) of SEQ ID NO: 7.

Anti-ILIRI Variable Heavy Chain (VH) Amino Acid
Sequence:
(SEQ ID NO: 7)
EVQLMQSGAEVKKPGESLKISCKGSGYSFSFHWIAWVRQMPGKGLEWMGI
IHPGASDTRYSPSFQGQVTISADNSNSATYLQWSSLKASDTAMYFCARQR
ELDYFDYWGQGTLVTVSS

In some embodiments, an anti-IL1R1 antibody comprises a VH that is at least 85% identical to SEQ ID NO: 7. In some embodiments, an anti-IL1R1 antibody comprises a VH that is at least 90% identical to SEQ ID NO: 7. In some embodiments, an anti-IL1R1 antibody comprises a VH that is at least 92% identical to SEQ ID NO: 7. In some embodiments, an anti-IL1R1 antibody comprises a VH that is at least 93% identical to SEQ ID NO: 7. In some embodiments, an anti-IL1R1 antibody comprises a VH that is at least 94% identical to SEQ ID NO: 7. In some embodiments, an anti-IL1R1 antibody comprises a VH that is at least 95% identical to SEQ ID NO: 7. In some embodiments, an anti-IL1R1 antibody comprises a VH that is at least 96% identical to SEQ ID NO: 7. In some embodiments, an anti-IL1R1 antibody comprises a VH that is at least 97% identical to SEQ ID NO: 7. In some embodiments, an anti-IL1R1 antibody comprises a VH that is at least 98% identical to SEQ ID NO: 7. In some embodiments, an anti-IL1R1 antibody comprises a VH that is at least 99% identical to SEQ ID NO: 7. In some embodiments, an anti-IL1R1 antibody comprises a VH of SEQ ID NO: 7.

In some embodiments, an anti-IL1R antibody comprises a HCDR1, a HCDR2, and a HCDR3 in the VH of SEQ ID NO: 7, as shown by underlining, respectively:

(SEQ ID NO: 7)
EVQLMQSGAEVKKPGESLKISCKGSGYSFSFHWIAWVRQMPGKGLEWMGI
IHPGASDTRYSPSFQGQVTISADNSNSATYLQWSSLKASDTAMYFCARQR
ELDYFDYWGQGTLVTVSS

In some embodiments, an anti-IL1R antibody comprises a HCDR1, a HCDR2, and a HCDR3 in the VH of SEQ ID NO: 7, as shown by underlining, respectively:

(SEQ ID NO: 7)
EVQLMQSGAEVKKPGESLKISCKGSGYSFSFHWIAWVRQMPGKGLEWMGI
IHPGASDTRYSPSFQGQVTISADNSNSATYLQWSSLKASDTAMYFCARQR
ELDYFDYWGQGTLVTVSS

In some embodiments, an anti-IL1R antibody comprises a HCDR1, a HCDR2, and a HCDR3 in the VH of SEQ ID NO: 7, as shown by underlining, respectively:

(SEQ ID NO: 7)
EVQLMQSGAEVKKPGESLKISCKGSGYSFSFHWIAWVRQMPGKGLEWMGI
IHPGASDTRYSPSFQGQVTISADNSNSATYLQWSSLKASDTAMYFCARQR
ELDYFDYWGQGTLVTVSS

In some embodiments, an anti-IL1R1 antibody comprises a variable light chain (VL) of SEQ ID NO: 8.

Anti-ILIRI Variable Light Chain (VL) Amino Acid
Sequence:
(SEQ ID NO: 8)
EIVLTQSPDFQSVTPKEKVTITCRASQSIGSSLHWYQQKPDQSPKLLIKY
ASQSFSGVPSRFSGSGSGTDFTLTINSLEAEDAAAYYCHQSSSLPLTFGG
GTKVEIK

In some embodiments, an anti-IL1R1 antibody comprises a VL that is at least 85% identical to SEQ ID NO: 8. In some embodiments, an anti-IL1R1 antibody comprises a VL that is at least 90% identical to SEQ ID NO: 8. In some embodiments, an anti-IL1R1 antibody comprises a VL that is at least 92% identical to SEQ ID NO: 8. In some embodiments, an anti-IL1R1 antibody comprises a VL that is at least 93% identical to SEQ ID NO: 8. In some embodiments, an anti-IL1R1 antibody comprises a VL that is at least 94% identical to SEQ ID NO: 8. In some embodiments, an anti-IL1R1 antibody comprises a VL that is at least 95% identical to SEQ ID NO: 8. In some embodiments, an anti-IL1R1 antibody comprises a VL that is at least 96% identical to SEQ ID NO: 8. In some embodiments, an anti-IL1R1 antibody comprises a VL that is at least 97% identical to SEQ ID NO: 8. In some embodiments, an anti-IL1R1 antibody comprises a VL that is at least 98% identical to SEQ ID NO: 8. In some embodiments, an anti-IL1R1 antibody comprises a VL that is at least 99% identical to SEQ ID NO: 8. In some embodiments, an anti-IL1R1 antibody comprises a VL of SEQ ID NO: 8.

In some embodiments, an anti-IL1R antibody comprises a LCDR1, a LCDR2, and a LCDR3 in the VL of SEQ ID NO: 8, as shown by underlining, respectively:

(SEQ ID NO: 8)
EIVLTQSPDFQSVTPKEKVTITCRASQSIGSSLHWYQQKPDQSPKLLIKY
ASQSFSGVPSRFSGSGSGTDFTLTINSLEAEDAAAYYCHQSSSLPLTFGG
GTKVEIK

In some embodiments, an anti-IL1R antibody comprises a LCDR1, a LCDR2, and a LCDR3 in the VL of SEQ ID NO: 8, as shown by underlining, respectively:

(SEQ ID NO: 8)
EIVLTQSPDFQSVTPKEKVTITCRASQSIGSSLHWYQQKPDQSPKLLIKY
ASQSFSGVPSRFSGSGSGTDFTLTINSLEAEDAAAYYCHQSSSLPLTFGG
GTKVEIK

### Anti-IL1R1 CDR Amino Acid Sequences

In some embodiments, an anti-IL1R1 antibody comprises a HCDR1, a HCDR2, and a HCDR3 as shown in any one of Tables 2-4. In some embodiments, an anti-IL1R1 antibody comprises a LCDR1, a LCDR2, and a LCDR3 as shown in any one of Tables 2-4.

In some embodiments, an anti-IL1R1 antibody comprises a HCDR1, a HCDR2, a HCDR3, a LCDR1, a LCDR2, and a LCDR3 as shown in any one of Tables 2-4.

TABLE 2
CDR Sequence of Anti-IL1R1 Antibody by Kabat
Numbering

| CDR | Amino acid sequence | SEQ ID NO |
|---|---|---|
| HCDR1 | FHWIA | SEQ ID NO: 1 |
| HCDR2 | IIHPGASDTRYSPSFQG | SEQ ID NO: 2 |
| HCDR3 | QRELDYFDY | SEQ ID NO: 3 |
| LCDR1 | RASQSIGSSLH | SEQ ID NO: 4 |
| LCDR2 | YASQSFS | SEQ ID NO: 5 |
| LCDR3 | HQSSSLPLT | SEQ ID NO: 6 |

TABLE 3
CDR Sequence of Anti-ILIR1 Antibody by Chothia
Numbering

| CDR | Amino acid sequence | SEQ ID NO |
|---|---|---|
| HCDR1 | GYSFSFH | SEQ ID NO: 12 |
| HCDR2 | HPGASD | SEQ ID NO: 13 |
| HCDR3 | QRELDYFDY | SEQ ID NO: 3 |
| LCDR1 | RASQSIGSSLH | SEQ ID NO: 4 |
| LCDR2 | YASQSFS | SEQ ID NO: 5 |
| LCDR3 | HQSSSLPLT | SEQ ID NO: 6 |

TABLE 4
CDR Sequence of Anti-ILIR1 Antibody by IMGT
Numbering

| CDR | Amino acid sequence | SEQ ID NO |
|---|---|---|
| HCDR1 | GYSFSFHW | SEQ ID NO: 14 |
| HCDR2 | IHPGASDT | SEQ ID NO: 15 |
| HCDR3 | ARQRELDYFDY | SEQ ID NO: 19 |
| LCDR1 | QSIGSS | SEQ ID NO: 16 |
| LCDR2 | YAS | SEQ ID NO: 17 |
| LCDR3 | HQSSSLPLT | SEQ ID NO: 6 |

In some embodiments, an anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, an anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In some embodiments, an anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In some embodiments, an anti-IL1R1 antibody comprises a HCDR1, a HCDR2, and a HCDR3 comprising amino acid residues 31-35, 50-65, and 95-102, respectively, of SEQ ID NO: 7 according to Kabat numbering. In some embodiments, an anti-IL1R1 antibody comprises a LCDR1, a LCDR2, and a LCDR3 comprising amino acid residues 24-34, 50-56, and 89-97, respectively, of SEQ ID NO: 8 according to Kabat numbering.

In some embodiments, an anti-IL1R1 antibody comprises a HCDR1, a HCDR2, and a HCDR3 comprising amino acid residues 27-35, 52-56, and 95-102, respectively, of SEQ ID NO: 7 according to Chothia numbering. In some embodiments, an anti-IL1R1 antibody comprises a LCDR1, a LCDR2, and a LCDR3 comprising amino acid residues 24-34, 50-56, and 89-97, respectively, of SEQ ID NO: 8 according to Chothia numbering.

In some embodiments, an anti-IL1R1 antibody comprises a HCDR1, a HCDR2, and a HCDR3 comprising amino acid residues 27-38, 56-65, and 105-117, respectively, of SEQ ID NO: 7 according to IMGT numbering. In some embodiments, an anti-IL1R1 antibody comprises a LCDR1, a LCDR2, and a LCDR3 comprising amino acid residues 27-38, 56-65, and 105-117, respectively, of SEQ ID NO: 8 according to IMGT numbering.

In some embodiments, an anti-IL1R1 antibody comprises a HCDR1, a HCDR2, and a HCDR3 amino acid sequences in SEQ ID NO: 7 as determined by any convention known in the art. In some embodiments, an anti-IL1R1 antibody comprises a LCDR1, a LCDR2, and a LCDR3 amino acid sequences in SEQ ID NO: 8 as determined by any convention known in the art.

Additional anti-IL1R1 antibodies are described in International Patent Publications WO 2010/052505, WO 2005/023872, and WO 2004/022718, incorporated herein by reference. In some embodiments, an anti-IL1R1 antibody comprises a HCDR1, a HCDR2, a HCDR3, a LCDR1, a LCDR2, and a LCDR3 as shown in Table 5.

TABLE 5
CDR Sequences of Exemplary Anti-ILIR1 Antibodies.

| Ab | HCDR1 | HCDR2 | HCDR3 | LCDR1 | LCDR2 | LCDR3 |
|---|---|---|---|---|---|---|
| Ab1 | SYAMS | AISGSGGST | DGASSTNWG | SGSSSNIGS | WNNQR | AAWDDSLS |
| | (SEQ ID | YYADSVKG | YNYYGMDV | DYVF | PS | GLV |
| | NO: 20) | (SEQ ID NO: | (SEQ ID NO: | (SEQ ID NO: | (SEQ ID | (SEQ ID NO: |
| | | 21) | 22) | 23) | NO: 24) | 25) |
| Ab2 | SYAMS | AISGSGGST | DGASSTNWG | SGSSSNIGS | WNNQR | AAWDDHLE |
| | (SEQ ID | YYADSVKG | YTVDAAVD | DYVF | PS | QLH |
| | NO: 20) | (SEQ ID NO: | (SEQ ID NO: | (SEQ ID NO: | (SEQ ID | (SEQ ID NO: |
| | | 21) | 26) | 23) | NO: 24) | 27) |
| Ab3 | SYAMS | AISGSGGST | DGASSTNWG | SGSSSNIGS | WNNQR | AAWDDAAA |
| | (SEQ ID | YYADSVKG | YTLDPPGV | NYVF | PS | RVLL |
| | NO: 20) | (SEQ ID NO: | (SEQ ID NO: | (SEQ ID NO: | (SEQ ID | (SEQ ID NO: |
| | | 21 | 28) | 45) | NO: 24) | 29) |
| Ab4 | SYAMS | AISGSGGST | PLYYYDGSD | TGSSSNIGA | GDTHR | QSYDTSLSG |
| | (SEQ ID | YYADSVKG | YTTYDAFDI | GYDVH | PS | SL |
| | NO: 20) | (SEQ ID NO: | (SEQ ID NO: | (SEQ ID NO: | (SEQ ID | (SEQ ID NO: |
| | | 21) | 30) | 31) | NO: 32) | 33) |
| Ab5 | SYAMS | AISGSGGST | PLYYYDAPPP | TGSSSNIGA | GDTHR | QSYDTAGG |
| | (SEQ ID | YYADSVKG | LGYDGFDI | GYDVH | PS | GHH |
| | NO: 20) | (SEQ ID NO: | (SEQ ID NO: | (SEQ ID NO: | (SEQ ID | (SEQ ID NO: |
| | | 21) | 30) | 31) | NO: 32) | 46) |
| Ab6 | SYAMS | AISGSGGST | PLYYYDQQY | TGSSSNIGA | GDTHR | QSYDVRLH |
| | (SEQ ID | YYADSVKG | GVVYDAFG | GYDVH | PS | HV |
| | NO: 20) | (SEQ ID NO: | (SEQ ID NO: | (SEQ ID NO: | (SEQ ID | (SEQ ID NO: |
| | | 21) | 34) | 31) | NO: 32) | 35) |
| Ab7 | SYMAS | AISGSGGST | PLYYYDQQY | TGSSSNIGA | GDTHR | QSYDTVRLH |
| | (SEQ ID | YYADSVKG | GVVYDAFV | GYDVH | PS | HV |
| | NO: 47) | (SEQ ID NO: | (SEQ ID NO: | (SEQ ID NO: | (SEQ ID | (SEQ ID NO: |
| | | 21) | 36) | 31) | NO: 32) | 48) |
| Ab8 | SYAMS | AISGSGGST | PLYYYDAAPP | TGSSSNIGA | GDTHR | QSYDTDAA |
| | (SEQ ID | YYADSVKG | LGYDGFDI | GYDVH | PS | RHQ |
| | NO: 20) | (SEQ ID NO: | (SEQ ID NO: | (SEQ ID NO: | (SEQ ID | (SEQ ID NO: |
| | | 21) | 37) | 31) | NO: 32) | 38) |
| Ab9 | SYAMS | AISGSGGST | PLYYYDAPSP | TGSSSNIGA | GDTHR | QSYDTHLVA |
| | (SEQ ID | YYADSVKG | LGYDGFDI | GYDVH | PS | HV |
| | NO: 20) | (SEQ ID NO: | (SEQ ID NO: | (SEQ ID NO: | (SEQ ID | (SEQ ID NO: |
| | | 21) | 39) | 31) | NO: 32) | 40) |
| Ab10 | SYAMS | AISGSGGST | PLYYYDEQY | TGSSSNIGA | GDTHR | QSYDTLLLA |
| | (SEQ ID | YYADSVKG | GLVYDAFDI | GYDVH | PS | PQ |
| | NO: 20) | (SEQ ID NO: | (SEQ ID NO: | (SEQ ID NO: | (SEQ ID | (SEQ ID NO: |
| | | 21) | 41) | 31) | NO: 32) | 42) |
| Ab11 | SYAMS | AISGSGGST | PLYYYDESLA | TGSSSNIGA | GDTHR | QSYDTRAD |
| | (SEQ ID | YYADSVKG | LPVYDADI | GYDVH | PS | DAH |
| | NO: 20) | (SEQ ID NO: | (SEQ ID NO: | (SEQ ID NO: | (SEQ ID | (SEQ ID NO: |
| | | 21) | 43) | 31) | NO: 32) | 44) |
| Ab12 | SNSIT | MIWSNGDT | YNYYFDY | KASQNVDN | KGSNL | QQSKGHPDT |
| | (SEQ ID | DYNSAFTS | (SEQ ID NO: | RGVSYVH | AF | |
| | NO: 49) | (SEQ ID NO: | 51) | (SEQ ID NO: | (SEQ ID | (SEQ ID NO: |
| | | 50) | | 52) | NO: 53) | 54) |
| Ab13 | NYGM | GIWNDGIN | ARSFDWLLFE | RASQSVSSY | DASNR | QQRSNWPPL |
| | H | KYHAHSVR | F | LA | AT | T |
| | (SEQ ID | G | (SEQ ID NO: | (SEQ ID NO: | (SEQ ID | (SEQ ID NO: |
| | NO: 55) | (SEQ ID NO: | 57) | 58) | NO: 59) | 60) |
| | | 56) | | | | |
| Ab14 | NYGM | AIWNDGEN | GRYFDWLLF | RASQSVSSY | DASNR | QQRSNWPPL |
| | H | KHHAGSVR | EY | LA | AT | T |
| | (SEQ ID | G | (SEQ ID NO: | (SEQ ID NO: | (SEQ ID | (SEQ ID NO: |
| | NO: 55) | (SEQ ID NO: | 62) | 58) | NO: 59) | 60) |
| | | 61) | | | | |

In some embodiments, an anti-IL1R1 antibody comprises a HCDR1 of SEQ ID NO: 20, a HCDR2 of SEQ ID NO: 21, a HCDR3 of SEQ ID NO: 22, a LCDR1 of SEQ ID NO: 23, a LCDR2 of SEQ ID NO: 24, and a LCDR3 of SEQ ID NO: 25.

In some embodiments, an anti-IL1R1 antibody comprises a HCDR1 of SEQ ID NO: 20, a HCDR2 of SEQ ID NO: 21, a HCDR3 of SEQ ID NO: 26, a LCDR1 of SEQ ID NO: 23, a LCDR2 of SEQ ID NO: 24, and a LCDR3 of SEQ ID NO: 27.

In some embodiments, an anti-IL1R1 antibody comprises a HCDR1 of SEQ ID NO: 20, a HCDR2 of SEQ ID NO: 21, a HCDR3 of SEQ ID NO: 28, a LCDR1 of SEQ ID NO: 45, a LCDR2 of SEQ ID NO: 24, and a LCDR3 of SEQ ID NO: 29.

In some embodiments, an anti-IL1R1 antibody comprises a HCDR1 of SEQ ID NO: 20, a HCDR2 of SEQ ID NO: 21, a HCDR3 of SEQ ID NO: 30, a LCDR1 of SEQ ID NO: 31, a LCDR2 of SEQ ID NO: 32, and a LCDR3 of SEQ ID NO: 33.

In some embodiments, an anti-IL1R1 antibody comprises a HCDR1 of SEQ ID NO: 20, a HCDR2 of SEQ ID NO: 21, a HCDR3 of SEQ ID NO: 30, a LCDR1 of SEQ ID NO: 31, a LCDR2 of SEQ ID NO: 32, and a LCDR3 of SEQ ID NO: 46.

In some embodiments, an anti-IL1R1 antibody comprises a HCDR1 of SEQ ID NO: 20, a HCDR2 of SEQ ID NO: 21, a HCDR3 of SEQ ID NO: 34, a LCDR1 of SEQ ID NO: 31, a LCDR2 of SEQ ID NO: 32, and a LCDR3 of SEQ ID NO: 35.

In some embodiments, an anti-IL1R1 antibody comprises a HCDR1 of SEQ ID NO: 47, a HCDR2 of SEQ ID NO: 21, a HCDR3 of SEQ ID NO: 36, a LCDR1 of SEQ ID NO: 31, a LCDR2 of SEQ ID NO: 32, and a LCDR3 of SEQ ID NO: 48.

In some embodiments, an anti-IL1R1 antibody comprises a HCDR1 of SEQ ID NO: 20, a HCDR2 of SEQ ID NO: 21, a HCDR3 of SEQ ID NO: 37, a LCDR1 of SEQ ID NO: 31, a LCDR2 of SEQ ID NO: 32, and a LCDR3 of SEQ ID NO: 38.

In some embodiments, an anti-IL1R1 antibody comprises a HCDR1 of SEQ ID NO: 20, a HCDR2 of SEQ ID NO: 21, a HCDR3 of SEQ ID NO: 39, a LCDR1 of SEQ ID NO: 31, a LCDR2 of SEQ ID NO: 32, and a LCDR3 of SEQ ID NO: 40.

In some embodiments, an anti-IL1R1 antibody comprises a HCDR1 of SEQ ID NO: 20, a HCDR2 of SEQ ID NO: 21, a HCDR3 of SEQ ID NO: 41, a LCDR1 of SEQ ID NO: 31, a LCDR2 of SEQ ID NO: 32, and a LCDR3 of SEQ ID NO: 42.

In some embodiments, an anti-IL1R1 antibody comprises a HCDR1 of SEQ ID NO: 20, a HCDR2 of SEQ ID NO: 21, a HCDR3 of SEQ ID NO: 41, a LCDR1 of SEQ ID NO: 31, a LCDR2 of SEQ ID NO: 32, and a LCDR3 of SEQ ID NO: 44.

In some embodiments, an anti-IL1R1 antibody comprises a HCDR1 of SEQ ID NO: 49, a HCDR2 of SEQ ID NO: 50, a HCDR3 of SEQ ID NO: 51, a LCDR1 of SEQ ID NO: 52, a LCDR2 of SEQ ID NO: 53, and a LCDR3 of SEQ ID NO: 54.

In some embodiments, an anti-IL1R1 antibody comprises a HCDR1 of SEQ ID NO: 55, a HCDR2 of SEQ ID NO: 56, a HCDR3 of SEQ ID NO: 57, a LCDR1 of SEQ ID NO: 58, a LCDR2 of SEQ ID NO: 59, and a LCDR3 of SEQ ID NO: 60.

In some embodiments, an anti-IL1R1 antibody comprises a HCDR1 of SEQ ID NO: 55, a HCDR2 of SEQ ID NO: 61, a HCDR3 of SEQ ID NO: 62, a LCDR1 of SEQ ID NO: 58, a LCDR2 of SEQ ID NO: 59, and a LCDR3 of SEQ ID NO: 60.

In some embodiments, an anti-IL1R1 antibody comprises an Fc region comprising an amino acid sequence of SEQ ID NO: 9.

Fc region of Anti-ILIRI Antibody:
(SEQ ID NO: 9)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

In some embodiments an Fc region comprises an amino acid sequence that is at least 85% identical to SEQ ID NO: 9. In some embodiments an Fc region comprises an amino acid sequence that is at least 90% identical to SEQ ID NO: 9. In some embodiments an Fc region comprises an amino acid sequence that is at least 92% identical to SEQ ID NO: 9. In some embodiments an Fc region comprises an amino acid sequence that is at least 93% identical to SEQ ID NO: 9. In some embodiments an Fc region comprises an amino acid sequence that is at least 94% identical to SEQ ID NO: 9. In some embodiments an Fc region comprises an amino acid sequence that is at least 95% identical to SEQ ID NO: 9. In some embodiments an Fc region comprises an amino acid sequence that is at least 96% identical to SEQ ID NO: 9. In some embodiments an Fc region comprises an amino acid sequence that is at least 97% identical to SEQ ID NO: 9. In some embodiments an Fc region comprises an amino acid sequence that is at least 98% identical to SEQ ID NO: 9. In some embodiments an Fc region comprises an amino acid sequence that is at least 99% identical to SEQ ID NO: 9.

In some embodiments, an anti-IL1R1 antibody comprises a heavy chain of SEQ ID NO: 10.

Anti-ILIRI Heavy Chain Amino Acid Sequence:
(SEQ ID NO: 10)
EVQLMQSGAEVKKPGESLKISCKGSGYSFSFHWIAWVRQMPGKGLEWMGI
IHPGASDTRYSPSFQGQVTISADNSNSATYLQWSSLKASDTAMYFCARQR
ELDYFDYWGQGTLVTVSSASTKGPSVFPLAPCSRSTSESTAALGCLVKDY
FPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMI
SRTPEVTCVVVDVSHEDPEVQFNWYVDGVEVHNAKTKPREEQFNSTFRVV
SVLTVVHQDWLNGKEYKCKVSNKGLPAPIEKTISKTKGQPREPQVYTLPP
SREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK

In some embodiments, an anti-IL1R1 antibody comprises a heavy chain that is at least 85% identical to SEQ ID NO: 10. In some embodiments, an anti-IL1R1 antibody comprises a heavy chain that is at least 90% identical to SEQ ID NO:10. In some embodiments, an anti-IL1R1 antibody comprises a heavy chain that is at least 92% identical to SEQ ID NO:10. In some embodiments, an anti-IL1R1 antibody comprises a heavy chain that is at least 93% identical to SEQ ID NO:10. In some embodiments, an anti-IL1R1 antibody comprises a heavy chain that is at least 94% identical to SEQ ID NO:10. In some embodiments, an anti-IL1R1 antibody comprises a heavy chain that is at least 95% identical to SEQ ID NO:10. In some embodiments, an anti-IL1R1 antibody comprises a heavy chain that is at least 96% identical to SEQ ID NO:10. In some embodiments, an anti-IL1R1 antibody comprises a heavy chain that is at least 97% identical to SEQ ID NO:10. In some embodiments, an anti-IL1R1 antibody comprises a heavy chain that is at least 98% identical to SEQ ID NO:10. In some embodiments, an anti-IL1R1 antibody comprises a heavy chain that is at least 99% identical to SEQ ID NO:10. In some embodiments, an anti-IL1R1 antibody comprises a heavy chain of SEQ ID NO:10.

In some embodiments, an anti-IL1R1 antibody comprises a light chain of SEQ ID NO: 11.

Anti-ILIRI Light Chain Amino Acid Sequence:
(SEQ ID NO: 11)
EIVLTQSPDFQSVTPKEKVTITCRASQSIGSSLHWYQQKPDQSPKLLIK
YASQSFSGVPSRFSGSGSGTDFTLTINSLEAEDAAAYYCHQSSSLPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, an anti-IL1R1 antibody comprises a light chain that is at least 85% identical to SEQ ID NO: 11. In some embodiments, an anti-IL1R1 antibody comprises a light chain that is at least 90% identical to SEQ ID NO: 11. In some embodiments, an anti-IL1R1 antibody comprises a light chain that is at least 92% identical to SEQ ID NO:11. In some embodiments, an anti-IL1R1 antibody comprises a light chain that is at least 93% identical to SEQ ID NO: 11. In some embodiments, an anti-IL1R1 antibody comprises a light chain that is at least 94% identical to SEQ ID NO: 11. In some embodiments, an anti-IL1R1 antibody comprises a light chain that is at least 95% identical to SEQ ID NO: 11. In some embodiments, an anti-IL1R1 antibody comprises a light chain that is at least 96% identical to SEQ ID NO: 11. In some embodiments, an anti-IL1R1 antibody comprises a light chain that is at least 97% identical to SEQ ID NO: 11. In some embodiments, an anti-IL1R1 antibody comprises a light chain that is at least 98% identical to SEQ ID NO: 11. In some embodiments, an anti-IL1R1 antibody comprises a light chain that is at least 99% identical to SEQ ID NO: 11. In some embodiments, an anti-IL1R1 antibody comprises a light chain of SEQ ID NO: 11.

### IL-1α and IL-1β Binding Proteins

IL-1α and IL-1β binding proteins also antagonize both IL-1α and IL-1β signaling and are beneficial for treating recurrent pericarditis according to methods described herein. Therefore, in the methods described herein, administration of an IL-1α and IL-1β binding protein (e.g., a dual variable domain binding protein that binds IL-1α and IL-1β) can be substituted for administration of an anti-IL1R1 antibody.

Dual variable domain binding proteins that bind IL-1α and IL-1β are described in U.S. Pat. No. 9,303,085 and U.S. Patent Application Publication 2015/0203592, each of which are incorporated herein by reference. In some embodiments, a dual variable domain binding protein that binds IL-1α and IL-1β comprises a light chain variable domain of SEQ ID NO: 63 and a heavy chain variable domain of SEQ ID NO: 64. A dual variable domain binding protein that binds IL-1α and IL-1β can comprise a heavy chain of SEQ ID NO: 65 and a light chain of SEQ ID NO: 66. In an embodiment, a dual variable domain binding protein that binds IL-1α and IL-1β is lutikizumab, which is also known as ABT-981.

TABLE 6
Amino Acid Sequences of an Exemplary Dual Variable Domain
Binding Protein that Binds IL-1α and IL-1β.

| | |
|---|---|
| Heavy chain | EVQLVESGGGVVQPGRSLRLSCSASGFIFSRYDMSWVRQAPGKGLE |
| variable domain | WVAYISHGGAGTYYPDSVKGRFTISRDNSKNTLFLQMDSLRPEDTGV |
| | YFCARGGVTKGYFDVWGQGTPVTVSSASTKGPQVQLVESGGGVVQP |
| | GRSLRLSCTASGFTFSMFGVHWVRQAPGKGLEWVAAVSYDGSNKY |
| | YAESVKGRFTISRDNSKNILFLQMDSLRLEDTAVYYCARGRPKVVIPA |
| | PLAHWGQGTLVTFSS (SEQ ID NO: 64) |
| Light chain | DIQMTQSPSSLSASVGDRVTITCRASGNIHNYLTWYQQTPGKAPKLLI |
| variable domain | YNAKTLADGVPSRFSGSGSGTDYTFTISSLQPEDIATYYCQHFWSIPYT |
| | FGQGTKLQITRTVAAPDIQMTQSPSSVSASVGDRVTITCRASQGISSW |
| | LAWYQQKPGKAPKLLIYEASNLETGVPSRFSGSGSGSDFTLTISSLQPE |
| | DFATYYCQQTSSFLLSFGGGTKVEHKR (SEQ ID NO: 63) |
| Heavy chain | EVQLVESGGGVVQPGRSLRLSCSASGFIFSRYDMSWVRQAPGKGLE |
| | WVAYISHGGAGTYYPDSVKGRFTISRDNSKNTLFLQMDSLRPEDTGV |
| | YFCARGGVTKGYFDVWGQGTPVTVSSASTKGPQVQLVESGGGVVQP |
| | GRSLRLSCTASGFTFSMFGVHWVRQAPGKGLEWVAAVSYDGSNKY |
| | YAESVKGRFTISRDNSKNILFLQMDSLRLEDTAVYYCARGRPKVVIPA |
| | PLAHWGQGTLVTFSSASTKGPSVFPLAPSSKSTSGGTAALGCLVKDYF |
| | PEPVTVSWNSGALTSGVHTFPAVLQSSGLYSLSSVVTVPSSSLGTQTYI |
| | CNVNHKPSNTKVDKKVEPKSCDKTHTCPPCPAPEAAGGPSVFLFPPK |
| | PKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALH |
| | NHYTQKSLSLSPGK (SEQ ID NO: 65) |
| Light chain | DIQMTQSPSSLSASVGDRVTITCRASGNIHNYLTWYQQTPGKAPKLLI |
| | YNAKTLADGVPSRFSGSGSGTDYTFTISSLQPEDIATYYCQHFWSIPYT |
| | FGQGTKLQITRTVAAPDIQMTQSPSSVSASVGDRVTITCRASQGISSW |
| | LAWYQQKPGKAPKLLIYEASNLETGVPSRFSGSGSGSDFTLTISSLQPE |
| | DFATYYCQQTSSFLLSFGGGTKVEHKRTVAAPSVFIFPPSDEQLKSGT |
| | ASVVCLLNNFYPREAKVQWKVDNALQSGNSQESVTEQDSKDSTYSL |
| | SSTLTLSKADYEKHKVYACEVTHQGLSSPVTKSFNRGEC (SEQ ID |
| | NO: 66) |

The dual variable domain binding proteins described above can be administered at a therapeutically effective dose. The dual variable binding proteins described above can be administered subcutaneously. In embodiments, the therapeutically effective dose is 250-500 mg. In embodiments, the therapeutically effective dose is 275-500 mg. In embodiments, the therapeutically effective dose is 300-500 mg. In embodiments, the therapeutically effective dose is 325-500 mg. In embodiments, the therapeutically effective dose is 350-500 mg. In embodiments, the therapeutically effective dose is 375-500 mg. In embodiments, the therapeutically effective dose is 400-500 mg. In embodiments, the therapeutically effective dose is 425-500 mg. In embodiments, the therapeutically effective dose is 450-500 mg. In embodiments, the therapeutically effective dose is 475-500 mg.

In embodiments, the therapeutically effective dose is 250-400 mg. In embodiments, the therapeutically effective dose is 250-375 mg. In embodiments, the therapeutically effective dose is 250-350 mg. In embodiments, the therapeutically effective dose is 250 to 325 mg. In embodiments, the therapeutically effective dose is 250-300 mg. In embodiments, the therapeutically effective dose is 150-300 mg. In embodiments, the therapeutically effective dose is 188-300 mg. In embodiments, the therapeutically effective dose is 225-300 mg. In embodiments, the therapeutically effective dose is 263-300 mg. In embodiments, the therapeutically effective dose is 275-300 mg. In embodiments, the therapeutically effective dose is 250-275 mg.

In embodiments, the therapeutically effective dose is 300-500 mg. In embodiments, the therapeutically effective dose is 300-475 mg. In embodiments, the therapeutically effective dose is 300-450 mg. In embodiments, the therapeutically effective dose is 300-425 mg. In embodiments, the therapeutically effective dose is 300-400 mg. In embodiments, the therapeutically effective dose is 300-375 mg. In embodiments, the therapeutically effective dose is 300-350 mg. In embodiments, the therapeutically effective dose is 300-325 mg.

In embodiments, the therapeutically effective dose is 188 mg. In embodiments, the therapeutically effective dose is 225 mg. In embodiments, the therapeutically effective dose is 250 mg. In embodiments, the therapeutically effective dose is 263 mg. In embodiments, the therapeutically effective dose is 275 mg. In embodiments, the therapeutically effective dose is 300 mg. In embodiments, the therapeutically effective dose is 325 mg. In embodiments, the therapeutically effective dose is 350 mg. In embodiments, the therapeutically effective dose is 375 mg. In embodiments, the therapeutically effective dose is 400 mg. In embodiments, the therapeutically effective dose is 425 mg. In embodiments, the therapeutically effective dose is 450 mg. In embodiments, the therapeutically effective dose is 475 mg. In embodiments, the therapeutically effective dose is 500 mg.

In embodiments, the binding protein is administered at any of the doses described above every week. For example, the binding protein can be administered at a dose of 300 mg every week. In embodiments, the binding protein is administered at any of the doses described above every two weeks. For example, the binding protein can be administered at a dose of 300 mg every two weeks.

In embodiments, the binding protein is administered every week for 16 weeks and every two weeks thereafter. For example, the binding protein can be administered at a dose of 300 mg every week for 16 weeks and at a dose of 300 mg every two weeks thereafter.

In one aspect, the present invention provides, among other things, a method of treating recurrent pericarditis, comprising administering subcutaneously to a patient a dual variable domain binding protein that binds IL-1α and IL-1β, e.g., lutikizumab, at a dose of 300 mg, wherein the dual variable domain binding protein is administered to the patient once every week or once every two weeks. In one embodiment, administration of a dual variable domain binding protein that binds IL-1α and IL-1β, e.g., lutikizumab, reduces the risk of pericarditis recurrence.

### Dosing Regimens

The present invention provides, among other things, a more efficacious and safer dosing regimens for treating recurrent pericarditis using an anti-IL1R1 antibody described herein. In some embodiments, a method comprises administering to a patient, an anti-interleukin-1 receptor type-1 (IL1R1) antibody at a therapeutically effective dose sufficient to improve, stabilize, prevent or reduce one or more symptoms of recurrent pericarditis relative to a control. In some embodiments, a method comprises administering to a patient, an anti-interleukin-1 receptor type-1 (IL1R1) antibody at a dose of greater than 10 mg. In some embodiments, a method comprises administering to a patient, an anti-interleukin-1 receptor type-1 (IL1R1) antibody at a therapeutically effective dose no more frequently than once every week.

### Dose

In some embodiments, the anti-IL1R1 antibody is administered at a dose of greater than 10 mg. In some embodiments, the anti-IL1R1 antibody is administered at a dose of greater than 15 mg. In some embodiments, the anti-IL1R1 antibody is administered at a dose of greater than 20 mg. In some embodiments, the anti-IL1R1 antibody is administered at a dose of greater than 25 mg. In some embodiments, the anti-IL1R1 antibody is administered at a dose of greater than 30 mg. In some embodiments, the anti-IL1R1 antibody is administered at a dose of greater than 35 mg. In some embodiments, the anti-IL1R1 antibody is administered at a dose of greater than 40 mg. In some embodiments, the anti-IL1R1 antibody is administered at a dose of greater than 45 mg. In some embodiments, the anti-IL1R1 antibody is administered at a dose of greater than 50 mg. In some embodiments, the anti-IL1R1 antibody is administered at a dose of greater than 55 mg. In some embodiments, the anti-IL1R1 antibody is administered at a dose of greater than 60 mg. In some embodiments, the anti-IL1R1 antibody is administered at a dose of greater than 65 mg. In some embodiments, the anti-IL1R1 antibody is administered at a dose of greater than 70 mg. In some embodiments, the anti-IL1R1 antibody is administered at a dose of greater than 75 mg. In some embodiments, the anti-IL1R1 antibody is administered at a dose of greater than 80 mg. In some embodiments, the anti-IL1R1 antibody is administered at a dose of greater than 85 mg. In some embodiments, the anti-IL1R1 antibody is administered at a dose of greater than 90 mg. In some embodiments, the anti-IL1R1 antibody is administered at a dose of greater than 95 mg. In some embodiments, the anti-IL1R1 antibody is administered at a dose of greater than 100 mg. In some embodiments, the anti-IL1R1 antibody is administered at a dose of greater than 110 mg. In some embodiments, the anti-IL1R1 antibody is administered at a dose of greater than 120 mg. In some embodiments, the anti-IL1R1 antibody is administered at a dose of greater than 130 mg. In some embodiments, the anti-IL1R1 antibody is administered at a dose of greater than 140 mg. In some embodiments, the anti-IL1R1 antibody is administered at a dose of greater than 150 mg. In some embodiments, the anti-IL1R1 antibody is administered at a dose of greater than 160 mg. In some embodiments, the anti-IL1R1 antibody is administered at a dose of greater than 170 mg. In some embodiments, the anti-IL1R1 antibody is administered at a dose of greater than 180 mg. In some embodiments, the anti-IL1R1 antibody is administered at a dose of greater than 188 mg. In some embodiments, the anti-IL1R1 antibody is administered at a dose of greater than 190 mg. In some embodiments, the anti-IL1R1 antibody is administered at a dose of greater than 200 mg. In some embodiments, the anti-IL1R1 antibody is administered at a dose of greater than 225 mg. In some embodiments, the anti-IL1R1 antibody is administered at a dose of greater than 250 mg. In some embodiments, the anti-IL1R1 antibody is administered at a dose of greater than 263 mg. In some embodiments, the anti-IL1R1 antibody is administered at a dose of greater than 275 mg. In some embodiments, the anti-IL1R1 antibody is administered at a dose of greater than 300 mg. In some embodiments, the anti-IL1R1 antibody is administered at a dose of greater than 400 mg. In some embodiments, the anti-IL1R1 antibody is administered at a dose of greater than 500 mg.

In some embodiments, the anti-IL1R1 antibody is administered at a dose of at least 5 mg. In some embodiments, the anti-IL1R1 antibody is administered at a dose of at least 10 mg. In some embodiments, the anti-IL1R1 antibody is administered at a dose of at least 15 mg. In some embodiments, the anti-IL1R1 antibody is administered at a dose of at least 20 mg. In some embodiments, the anti-IL1R1 antibody is administered at a dose of at least 25 mg. In some embodiments, the anti-IL1R1 antibody is administered at a dose of at least 30 mg. In some embodiments, the anti-IL1R1 antibody is administered at a dose of at least 40 mg. In some embodiments, the anti-IL1R1 antibody is administered at a dose of at least 50 mg. In some embodiments, the anti-IL1R1 antibody is administered at a dose of at least 60 mg. In some embodiments, the anti-IL1R1 antibody is administered at a dose of at least 70 mg. In some embodiments, the anti-IL1R1 antibody is administered at a dose of at least 80 mg. In some embodiments, the anti-IL1R1 antibody is administered at a dose of at least 90 mg. In some embodiments, the anti-IL1R1 antibody is administered at a dose of at least 100 mg. In some embodiments, the anti-IL1R1 antibody is administered at a dose of at least 110 mg. In some embodiments, the anti-IL1R1 antibody is administered at a dose of at least 125 mg. In some embodiments, the anti-IL1R1 antibody is administered at a dose of at least 150 mg. In some embodiments, the anti-IL1R1 antibody is administered at a dose of at least 175 mg. In some embodiments, the anti-IL1R1 antibody is administered at a dose of at least 200 mg. In some embodiments, the anti-IL1R1 antibody is administered at a dose of at least 250 mg. In some embodiments, the anti-IL1R1 antibody is administered at a dose of at least 300 mg. In some embodiments, the anti-IL1R1 antibody is administered at a dose of at least 350 mg. In some embodiments, the anti-IL1R1 antibody is administered at a dose of at least 400 mg. In some embodiments, the anti-IL1R1 antibody is administered at a dose of at least 500 mg.

In some embodiments, the anti-IL1R1 antibody is administered at a dose of between 10 mg and 1000 mg. In some embodiments, the anti-IL1R1 antibody is administered at a dose of between 10 mg and 500 mg. In some embodiments, the anti-IL1R1 antibody is administered at a dose of between 50 mg and 500 mg. In some embodiments, the anti-IL1R1 antibody is administered at a dose of between 50 mg and 400 mg. In some embodiments, the anti-IL1R1 antibody is administered at a dose of between 100 mg and 400 mg. In some embodiments, the anti-IL1R1 antibody is administered at a dose of between 75 and 350 mg. In some embodiments, the anti-IL1R1 antibody is administered at a dose of between 100 mg and 300 mg. In some embodiments, the anti-IL1R1 antibody is administered at a dose of between 10 and 100 mg. In some embodiments, the anti-IL1R1 antibody is administered at a dose of between 50 mg and 150 mg. In some embodiments, the anti-IL1R1 antibody is administered at a dose of between 75 mg and 125 mg. In some embodiments, the anti-IL1R1 antibody is administered at a dose of between 150 and 400 mg. In some embodiments, the anti-IL1R1 antibody is administered at a dose of between 150 and 300 mg. In some embodiments, the anti-IL1R1 antibody is administered at a dose of between 150 and 350 mg. In some embodiments, the anti-IL1R1 antibody is administered at a dose of between 175 mg and 325 mg.

In some embodiments, the anti-IL1R1 antibody is administered at a dose of 1 mg. In some embodiments, the anti-IL1R1 antibody is administered at a dose of 3 mg. In some embodiments, the anti-IL1R1 antibody is administered at a dose of 5 mg. In some embodiments, the anti-IL1R1 antibody is administered at a dose of 10 mg. In some embodiments, the anti-IL1R1 antibody is administered at a dose of 15 mg. In some embodiments, the anti-IL1R1 antibody is administered at a dose of 20 mg. In some embodiments, the anti-IL1R1 antibody is administered at a dose of 25 mg. In some embodiments, the anti-IL1R1 antibody is administered at a dose of 30 mg. In some embodiments, the anti-IL1R1 antibody is administered at a dose of 35 mg. In some embodiments, the anti-IL1R1 antibody is administered at a dose of 40 mg. In some embodiments, the anti-IL1R1 antibody is administered at a dose of 45 mg. In some embodiments, the anti-IL1R1 antibody is administered at a dose of 50 mg. In some embodiments, the anti-IL1R1 antibody is administered at a dose of 55 mg. In some embodiments, the anti-IL1R1 antibody is administered at a dose of 60 mg. In some embodiments, the anti-IL1R1 antibody is administered at a dose of 65 mg. In some embodiments, the anti-IL1R1 antibody is administered at a dose of 70 mg. In some embodiments, the anti-IL1R1 antibody is administered at a dose of 75 mg. In some embodiments, the anti-IL1R1 antibody is administered at a dose of 80 mg. In some embodiments, the anti-IL1R1 antibody is administered at a dose of 85 mg. In some embodiments, the anti-IL1R1 antibody is administered at a dose of 90 mg. In some embodiments, the anti-IL1R1 antibody is administered at a dose of 95 mg. In some embodiments, the anti-IL1R1 antibody is administered at a dose of 100 mg. In some embodiments, the anti-IL1R1 antibody is administered at a dose of 110 mg. In some embodiments, the anti-IL1R1 antibody is administered at a dose of 120 mg. In some embodiments, the anti-IL1R1 antibody is administered at a dose of 125 mg. In some embodiments, the anti-IL1R1 antibody is administered at a dose of 130 mg. In some embodiments, the anti-IL1R1 antibody is administered at a dose of 150 mg. In some embodiments, the anti-IL1R1 antibody is administered at a dose of 175 mg. In some embodiments, the anti-IL1R1 antibody is administered at a dose of 188 mg. In some embodiments, the anti-IL1R1 antibody is administered at a dose of 200 mg. In some embodiments, the anti-IL1R1 antibody is administered at a dose of 225 mg. In some embodiments, the anti-IL1R1 antibody is administered at a dose of 250 mg. In some embodiments, the anti-IL1R1 antibody is administered at a dose of 263 mg. In some embodiments, the anti-IL1R1 antibody is administered at a dose of 275 mg. In some embodiments, the anti-IL1R1 antibody is administered at a dose of 300 mg. In some embodiments, the anti-IL1R1 antibody is administered at a dose of 325 mg. In some embodiments, the anti-IL1R1 antibody is administered at a dose of 350 mg. In some embodiments, the anti-IL1R1 antibody is administered at a dose of 375 mg. In some embodiments, the anti-IL1R1 antibody is administered at a dose of 400 mg. In some embodiments, the anti-IL1R1 antibody is administered at a dose of 500 mg. In some embodiments, the anti-IL1R1 antibody is administered at a dose of 750 mg. In some embodiments, the anti-IL1R1 antibody is administered at a dose of 800 mg. In some embodiments, the anti-IL1R1 antibody is administered at a dose of 900 mg. In some embodiments, the anti-IL1R1 antibody is administered at a dose of 1000 mg.

### Administration Interval

In some embodiments, an anti-interleukin-1 receptor type-1 (IL1R1) antibody at a therapeutically effective dose at an administration interval sufficient to improve, stabilize, prevent or reduce one or more symptoms of recurrent pericarditis.

In some embodiments, an anti-IL1R1 antibody is administered no more frequently than once every 5 days. In some embodiments, an anti-IL1R1 antibody is administered no more frequently than once every 7 days. In some embodiments, an anti-IL1R1 antibody is administered no more frequently than once every 10 days. In some embodiments, an anti-IL1R1 antibody is administered no more frequently than once every 15 days. In some embodiments, an anti-IL1R1 antibody is administered no more frequently than once every 28 days. In some embodiments, an anti-IL1R1 antibody is administered no more frequently than once every week. In some embodiments, an anti-IL1R1 antibody is administered no more frequently than every other week. In some embodiments, an anti-IL1R1 antibody is administered no more frequently than once every 3 weeks. In some embodiments, an anti-IL1R1 antibody is administered no more frequently than once every 4 weeks. In some embodiments, an anti-IL1R1 antibody is administered no more frequently than once every 5 weeks. In some embodiments, an anti-IL1R1 antibody is administered no more frequently than once every 6 weeks. In some embodiments, an anti-IL1R1 antibody is administered no more frequently than once every 7 weeks. In some embodiments, an anti-IL1R1 antibody is administered no more frequently than once every 8 weeks. In some embodiments, an anti-IL1R1 antibody is administered no more frequently than once every month. In some embodiments, an anti-IL1R1 antibody is administered no more frequently than once every two months.

In some embodiments, an anti-IL1R1 antibody is administered once every 5 days. In some embodiments, an anti-IL1R1 antibody is administered once every 7 days. In some embodiments, an anti-IL1R1 antibody is administered once every 10 days. In some embodiments, an anti-IL1R1 antibody is administered once every 15 days. In some embodiments, an anti-IL1R1 antibody is administered once every 28 days. In some embodiments, an anti-IL1R1 antibody is administered once every week. In some embodiments, an anti-IL1R1 antibody is administered once every other week. In some embodiments, an anti-IL1R1 antibody is administered once every 3 weeks. In some embodiments, an anti-IL1R1 antibody is administered once every 4 weeks. In some embodiments, an anti-IL1R1 antibody is administered once every 5 weeks. In some embodiments, an anti-IL1R1 antibody is administered once every 6 weeks. In some embodiments, an anti-IL1R1 antibody is administered once every 7 weeks. In some embodiments, an anti-IL1R1 antibody is administered once every 8 weeks. In some embodiments, an anti-IL1R1 antibody is administered once every month. In some embodiments, an anti-IL1R1 antibody is administered once every two months.

In some embodiments, an anti-IL1R1 antibody is administered at a dose of greater than 10 mg once every 5 days. In some embodiments, an anti-IL1R1 antibody is administered at a dose of greater than 10 mg once every 7 days. In some embodiments, an anti-IL1R1 antibody is administered at a dose of greater than 10 mg once every 10 days. In some embodiments, an anti-IL1R1 antibody is administered at a dose of greater than 10 mg once every 15 days. In some embodiments, an anti-IL1R1 antibody is administered at a dose of greater than 10 mg once every 28 days. In some embodiments, an anti-IL1R1 antibody is administered at a dose of greater than 10 mg once every week. In some embodiments, an anti-IL1R1 antibody is administered at a dose of greater than 10 mg once every other week. In some embodiments, an anti-IL1R1 antibody is administered at a dose of greater than 10 mg once every 3 weeks. In some embodiments, an anti-IL1R1 antibody is administered at a dose of greater than 10 mg once every 4 weeks. In some embodiments, an anti-IL1R1 antibody is administered at a dose of greater than 10 mg once every 5 weeks. In some embodiments, an anti-IL1R1 antibody is administered at a dose of greater than 10 mg once every 6 weeks. In some embodiments, an anti-IL1R1 antibody is administered at a dose of greater than 10 mg once every 7 weeks. In some embodiments, an anti-IL1R1 antibody is administered at a dose of greater than 10 mg once every 8 weeks. In some embodiments, an anti-IL1R1 antibody is administered at a dose of greater than 10 mg once every month. In some embodiments, an anti-IL1R1 antibody is administered at a dose of greater than 10 mg once two months.

In some embodiments, an anti-IL1R1 antibody is administered at a dose of between 30 mg and 500 mg once every 5 days. In some embodiments, an anti-IL1R1 antibody is administered at a dose of between 30 mg and 500 mg once every 7 days. In some embodiments, an anti-IL1R1 antibody is administered at a dose of between 30 mg and 500 mg once every 10 days. In some embodiments, an anti-IL1R1 antibody is administered at a dose of between 30 mg and 500 mg once every 15 days. In some embodiments, an anti-IL1R1 antibody is administered at a dose of between 30 mg and 500 mg once every 28 days. In some embodiments, an anti-IL1R1 antibody is administered at a dose of between 30 mg and 500 mg once every week. In some embodiments, an anti-IL1R1 antibody is administered at a dose of between 30 mg and 500 mg once every other week. In some embodiments, an anti-IL1R1 antibody is administered at a dose of between 30 mg and 500 mg once every 3 weeks. In some embodiments, an anti-IL1R1 antibody is administered at a dose of between 30 mg and 500 mg once every 4 weeks. In some embodiments, an anti-IL1R1 antibody is administered at a dose of between 30 mg and 500 mg once every 5 weeks. In some embodiments, an anti-IL1R1 antibody is administered at a dose of between 30 mg and 500 mg once every 6 weeks. In some embodiments, an anti-IL1R1 antibody is administered at a dose of between 30 mg and 500 mg once every 7 weeks. In some embodiments, an anti-IL1R1 antibody is administered at a dose of between 30 mg and 500 mg once every 8 weeks. In some embodiments, an anti-IL1R1 antibody is administered at a dose of between 30 mg and 500 mg once every month. In some embodiments, an anti-IL1R1 antibody is administered at a dose of between 30 mg and 500 mg once two months.

In some embodiments, an anti-IL1R1 antibody is administered at a dose of between 100 mg and 300 mg once every 5 days. In some embodiments, an anti-IL1R1 antibody is administered at a dose of between 100 mg and 300 mg once every 7 days. In some embodiments, an anti-IL1R1 antibody is administered at a dose of between 100 mg and 300 mg once every 10 days. In some embodiments, an anti-IL1R1 antibody is administered at a dose of between 100 mg and 300 mg once every 15 days. In some embodiments, an anti-IL1R1 antibody is administered at a dose of between 100 mg and 300 mg once every 28 days. In some embodiments, an anti-IL1R1 antibody is administered at a dose of between 100 mg and 300 mg once every week. In some embodiments, an anti-IL1R1 antibody is administered at a dose of between 100 mg and 300 mg once every other week. In some embodiments, an anti-IL1R1 antibody is administered at a dose of between 100 mg and 300 mg once every 3 weeks. In some embodiments, an anti-IL1R1 antibody is administered at a dose of between 100 mg and 300 mg once every 4 weeks. In some embodiments, an anti-IL1R1 antibody is administered at a dose of between 100 mg and 300 mg once every 5 weeks. In some embodiments, an anti-IL1R1 antibody is administered at a dose of between 100 mg and 300 mg once every 6 weeks. In some embodiments, an anti-IL1R1 antibody is administered at a dose of between 100 mg and 300 mg once every 7 weeks. In some embodiments, an anti-IL1R1 antibody is administered at a dose of between 100 mg and 300 mg once every 8 weeks. In some embodiments, an anti-IL1R1 antibody is administered at a dose of between 100 mg and 300 mg once every month. In some embodiments, an anti-IL1R1 antibody is administered at a dose of between 100 mg and 300 mg once every two months.

In some embodiments, an anti-IL1R1 antibody is administered at a dose of 100 mg once every 5 days. In some embodiments, an anti-IL1R1 antibody is administered at a dose of 100 mg once every 7 days. In some embodiments, an anti-IL1R1 antibody is administered at a dose of 100 mg once every 10 days. In some embodiments, an anti-IL1R1 antibody is administered at a dose of 100 mg once every 15 days. In some embodiments, an anti-IL1R1 antibody is administered at a dose of 100 mg once every week. In some embodiments, an anti-IL1R1 antibody is administered at a dose of 100 mg once every other week. In some embodiments, an anti-IL1R1 antibody is administered at a dose of 100 mg once every 3 weeks. In some embodiments, an anti-IL1R1 antibody is administered at a dose of 100 mg once every 4 weeks. In some embodiments, an anti-IL1R1 antibody is administered at a dose of 100 mg once every 5 weeks. In some embodiments, an anti-IL1R1 antibody is administered at a dose of 100 mg once every 6 weeks. In some embodiments, an anti-IL1R1 antibody is administered at a dose of 100 mg once every 7 weeks. In some embodiments, an anti-IL1R1 antibody is administered at a dose of 100 mg once every 8 weeks. In some embodiments, an anti-IL1R1 antibody is administered at a dose of 100 mg once every month. In some embodiments, an anti-IL1R1 antibody is administered at a dose of 100 mg once every two months.

In some embodiments, an anti-IL1R1 antibody is administered at a dose of 300 mg once every 5 days. In some embodiments, an anti-IL1R1 antibody is administered at a dose of 300 mg once every 7 days. In some embodiments, an anti-IL1R1 antibody is administered at a dose of 300 mg once every 10 days. In some embodiments, an anti-IL1R1 antibody is administered at a dose of 300 mg once every 15 days. In some embodiments, an anti-IL1R1 antibody is administered at a dose of 300 mg once every 28 days. In some embodiments, an anti-IL1R1 antibody is administered at a dose of 300 mg once every week. In some embodiments, an anti-IL1R1 antibody is administered at a dose of 300 mg once every other week. In some embodiments, an anti-IL1R1 antibody is administered at a dose of 300 mg once every 3 weeks. In some embodiments, an anti-IL1R1 antibody is administered at a dose of 300 mg once every 4 weeks. In some embodiments, an anti-IL1R1 antibody is administered at a dose of 300 mg once every 5 weeks. In some embodiments, an anti-IL1R1 antibody is administered at a dose of 300 mg once every 6 weeks. In some embodiments, an anti-IL1R1 antibody is administered at a dose of 300 mg once every 7 weeks. In some embodiments, an anti-IL1R1 antibody is administered at a dose of 300 mg once every 8 weeks. In some embodiments, an anti-IL1R1 antibody is administered at a dose of 300 mg once every month. In some embodiments, an anti-IL1R1 antibody is administered at a dose of 300 mg once every 2 months.

In some embodiments, the anti-IL1R1 antibody is administered to the patient at a dose of 1 mg/kg. In some embodiments, the anti-IL1R1 antibody is administered to the patient at a dose of 1.5 mg/kg. In some embodiments, the anti-IL1R1 antibody is administered to the patient at a dose of 2 mg/kg. In some embodiments, the anti-IL1R1 antibody is administered to the patient at a dose of 2.5 mg/kg. In some embodiments, the anti-IL1R1 antibody is administered to the patient at a dose of 3 mg/kg. In some embodiments, the anti-IL1R1 antibody is administered to the patient at a dose of 3.5 mg/kg. In some embodiments, the anti-IL1R1 antibody is administered to the patient at a dose of 4 mg/kg. In some embodiments, the anti-IL1R1 antibody is administered to the patient at a dose of 4.5 mg/kg. In some embodiments, the anti-IL1R1 antibody is administered to the patient at a dose of 5 mg/kg. In some embodiments, the anti-IL1R1 antibody is administered to the patient at a dose of 5.5 mg/kg. In some embodiments, the anti-IL1R1 antibody is administered to the patient at a dose of 6 mg/kg. In some embodiments, the anti-IL1R1 antibody is administered to the patient at a dose of 6.5 mg/kg. In some embodiments, the anti-IL1R1 antibody is administered to the patient at a dose of 7 mg/kg. In some embodiments, the anti-IL1R1 antibody is administered to the patient at a dose of 7.5 mg/kg. In some embodiments, the anti-IL1R1 antibody is administered to the patient at a dose of 8 mg/kg. In some embodiments, the anti-IL1R1 antibody is administered to the patient at a dose of 8.5 mg/kg. In some embodiments, the anti-IL1R1 antibody is administered to the patient at a dose of 9 mg/kg. In some embodiments, the anti-IL1R1 antibody is administered to the patient at a dose of 9.5 mg/kg. In some embodiments, the anti-IL1R antibody is administered to the patient at a dose of 10 mg/kg.

In some embodiments, the anti-IL1R antibody is administered to the patient at a dose of 1 mg/kg once every two weeks. In some embodiments, the anti-IL1R1 antibody is administered to the patient at a dose of 1.5 mg/kg once every two weeks. In some embodiments, the anti-IL1R1 antibody is administered to the patient at a dose of 2 mg/kg once every two weeks. In some embodiments, the anti-IL1R antibody is administered to the patient at a dose of 2.5 mg/kg once every two weeks. In some embodiments, the anti-IL1R antibody is administered to the patient at a dose of 3 mg/kg once every two weeks. In some embodiments, the anti-IL1R1 antibody is administered to the patient at a dose of 3.5 mg/kg once every two weeks. In some embodiments, the anti-IL1R1 antibody is administered to the patient at a dose of 4 mg/kg once every two weeks. In some embodiments, the anti-IL1R1 antibody is administered to the patient at a dose of 4.5 mg/kg once every two weeks. In some embodiments, the anti-IL1R1 antibody is administered to the patient at a dose of 5 mg/kg once every two weeks. In some embodiments, the anti-IL1R1 antibody is administered to the patient at a dose of 5.5 mg/kg once every two weeks. In some embodiments, the anti-IL1R1 antibody is administered to the patient at a dose of 6 mg/kg once every two weeks. In some embodiments, the anti-IL1R1 antibody is administered to the patient at a dose of 6.5 mg/kg once every two weeks. In some embodiments, the anti-IL1R1 antibody is administered to the patient at a dose of 7 mg/kg once every two weeks. In some embodiments, the anti-IL1R1 antibody is administered to the patient at a dose of 7.5 mg/kg once every two weeks. In some embodiments, the anti-IL1R1 antibody is administered to the patient at a dose of 8 mg/kg once every two weeks. In some embodiments, the anti-IL1R1 antibody is administered to the patient at a dose of 8.5 mg/kg once every two weeks. In some embodiments, the anti-IL1R1 antibody is administered to the patient at a dose of 9 mg/kg once every two weeks. In some embodiments, the anti-IL1R1 antibody is administered to the patient at a dose of 9.5 mg/kg once every two weeks. In some embodiments, the anti-IL1R1 antibody is administered to the patient at a dose of 10 mg/kg once every two weeks.

In some embodiments, the anti-IL1R1 antibody is administered to the patient at a dose of 1 mg/kg once every four weeks. In some embodiments, the anti-IL1R1 antibody is administered to the patient at a dose of 1.5 mg/kg once every four weeks. In some embodiments, the anti-IL1R1 antibody is administered to the patient at a dose of 2 mg/kg once every four weeks. In some embodiments, the anti-IL1R1 antibody is administered to the patient at a dose of 2.5 mg/kg once every four weeks. In some embodiments, the anti-IL1R1 antibody is administered to the patient at a dose of 3 mg/kg once every four weeks. In some embodiments, the anti-IL1R1 antibody is administered to the patient at a dose of 3.5 mg/kg once every four weeks. In some embodiments, the anti-IL1R1 antibody is administered to the patient at a dose of 4 mg/kg once every four weeks. In some embodiments, the anti-IL1R1 antibody is administered to the patient at a dose of 4.5 mg/kg once every four weeks. In some embodiments, the anti-IL1R1 antibody is administered to the patient at a dose of 5 mg/kg once every four weeks. In some embodiments, the anti-IL1R1 antibody is administered to the patient at a dose of 5.5 mg/kg once every four weeks. In some embodiments, the anti-IL1R1 antibody is administered to the patient at a dose of 6 mg/kg once every four weeks. In some embodiments, the anti-IL1R1 antibody is administered to the patient at a dose of 6.5 mg/kg once every four weeks. In some embodiments, the anti-IL1R1 antibody is administered to the patient at a dose of 7 mg/kg once every four weeks. In some embodiments, the anti-IL1R1 antibody is administered to the patient at a dose of 7.5 mg/kg once every four weeks. In some embodiments, the anti-IL1R1 antibody is administered to the patient at a dose of 8 mg/kg once every four weeks. In some embodiments, the anti-IL1R1 antibody is administered to the patient at a dose of 8.5 mg/kg once every four weeks. In some embodiments, the anti-IL1R1 antibody is administered to the patient at a dose of 9 mg/kg once every four weeks. In some embodiments, the anti-IL1R1 antibody is administered to the patient at a dose of 9.5 mg/kg once every four weeks. In some embodiments, the anti-IL1R1 antibody is administered to the patient at a dose of 10 mg/kg once every four weeks.

In one aspect, the present invention provides, among other things, a method of treating recurrent pericarditis comprising administering to a patient, an anti-interleukin-1 receptor type-1 (IL1R1) antibody at a dose of 100 mg once every two weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 100 mg once every two weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every two weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every two weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every two weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 100 mg once every four weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 100 mg once every four weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every four weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every four weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 100 mg once every four weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 150 mg once every two weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 150 mg once every two weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every two weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every two weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every two weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 150 mg once every four weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 150 mg once every four weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every four weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every four weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 150 mg once every four weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 225 mg once every two weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 225 mg once every two weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every two weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every two weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every two weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 225 mg once every four weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 225 mg once every four weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every four weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every four weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 225 mg once every four weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 250 mg once every two weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 250 mg once every two weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every two weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every two weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every two weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 250 mg once every four weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 250 mg once every four weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every four weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every four weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 250 mg once every four weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 300 mg once every two weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 300 mg once every two weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every two weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every two weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every two weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

In one aspect, the present invention provides, among other things, a method of treating recurrent pericarditis comprising administering to a patient, an anti-interleukin-1 receptor type-1 (IL1R1) antibody at a dose of 300 mg once every four weeks, or an equivalent dosing regimen thereof.

In one aspect, the present invention provides, among other things, a method of treating recurrent pericarditis comprising administering to a patient, an anti-interleukin-1 receptor type-1 (IL1R1) antibody at a dose of 300 mg once every four weeks, or an equivalent dosing regimen thereof, wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every four weeks, or an equivalent dosing regimen thereof, wherein the anti-IL1R1 antibody comprises a HCDR1 of SEQ ID NO: 12, a HCDR2 of SEQ ID NO: 13, a HCDR3 of SEQ ID NO: 3, a LCDR1 of SEQ ID NO: 4, a LCDR2 of SEQ ID NO: 5, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every four weeks, or an equivalent dosing regimen thereof, wherein the anti-IL1R1 antibody comprises a HCDR1 of SEQ ID NO: 14, a HCDR2 of SEQ ID NO: 15, a HCDR3 of SEQ ID NO: 19, a LCDR1 of SEQ ID NO: 16, a LCDR2 of SEQ ID NO: 17, and a LCDR3 of SEQ ID NO: 6.

In one aspect, the present invention provides, among other things, a method of treating recurrent pericarditis comprising administering to a patient, an anti-interleukin-1 receptor type-1 (IL1R1) antibody at a dose of 300 mg once every four weeks, or an equivalent dosing regimen thereof, wherein the anti-IL1R1 antibody comprises a HCDR1, a HCDR2, and a HCDR3 in the variable heavy chain (VH) of SEQ ID NO: 7 and a LCDR1, a LCDR2, and a LCDR3 of the variable light chain (VL) of SEQ ID NO: 8.

### Pediatric

In some embodiments, the anti-IL1R1 antibody is administered to the pediatric patient at a dose of 1 mg/kg. In some embodiments, the anti-IL1R1 antibody is administered to the pediatric patient at a dose of 1.5 mg/kg. In some embodiments, the anti-IL1R1 antibody is administered to the pediatric patient at a dose of 2 mg/kg. In some embodiments, the anti-IL1R1 antibody is administered to the pediatric patient at a dose of 2.5 mg/kg. In some embodiments, the anti-IL1R1 antibody is administered to the pediatric patient at a dose of 3 mg/kg. In some embodiments, the anti-IL1R1 antibody is administered to the pediatric patient at a dose of 3.5 mg/kg. In some embodiments, the anti-IL1R1 antibody is administered to the pediatric patient at a dose of 4 mg/kg. In some embodiments, the anti-IL1R1 antibody is administered to the pediatric patient at a dose of 4.5 mg/kg. In some embodiments, the anti-IL1R1 antibody is administered to the pediatric patient at a dose of 5 mg/kg. In some embodiments, the anti-IL1R1 antibody is administered to the pediatric patient at a dose of 5.5 mg/kg. In some embodiments, the anti-IL1R1 antibody is administered to the pediatric patient at a dose of 6 mg/kg. In some embodiments, the anti-IL1R1 antibody is administered to the pediatric patient at a dose of 6.5 mg/kg. In some embodiments, the anti-IL1R1 antibody is administered to the pediatric patient at a dose of 7 mg/kg. In some embodiments, the anti-IL1R1 antibody is administered to the pediatric patient at a dose of 7.5 mg/kg. In some embodiments, the anti-IL1R1 antibody is administered to the pediatric patient at a dose of 8 mg/kg. In some embodiments, the anti-IL1R1 antibody is administered to the pediatric patient at a dose of 8.5 mg/kg. In some embodiments, the anti-IL1R1 antibody is administered to the pediatric patient at a dose of 9 mg/kg. In some embodiments, the anti-IL1R1 antibody is administered to the pediatric patient at a dose of 9.5 mg/kg. In some embodiments, the anti-IL1R1 antibody is administered to the pediatric patient at a dose of 10 mg/kg.

In some embodiments, the anti-IL1R1 antibody is administered to the pediatric patient at a dose of 1 mg/kg once every two weeks. In some embodiments, the anti-IL1R1 antibody is administered to the pediatric patient at a dose of 1.5 mg/kg once every two weeks. In some embodiments, the anti-IL1R1 antibody is administered to the pediatric patient at a dose of 2 mg/kg once every two weeks. In some embodiments, the anti-IL1R1 antibody is administered to the pediatric patient at a dose of 2.5 mg/kg once every two weeks. In some embodiments, the anti-IL1R1 antibody is administered to the pediatric patient at a dose of 3 mg/kg once every two weeks. In some embodiments, the anti-IL1R1 antibody is administered to the pediatric patient at a dose of 3.5 mg/kg once every two weeks. In some embodiments, the anti-IL1R1 antibody is administered to the pediatric patient at a dose of 4 mg/kg once every two weeks. In some embodiments, the anti-IL1R1 antibody is administered to the pediatric patient at a dose of 4.5 mg/kg once every two weeks. In some embodiments, the anti-IL1R1 antibody is administered to the pediatric patient at a dose of 5 mg/kg once every two weeks. In some embodiments, the anti-IL1R1 antibody is administered to the pediatric patient at a dose of 5.5 mg/kg once every two weeks. In some embodiments, the anti-IL1R1 antibody is administered to the pediatric patient at a dose of 6 mg/kg once every two weeks. In some embodiments, the anti-IL1R1 antibody is administered to the pediatric patient at a dose of 6.5 mg/kg once every two weeks. In some embodiments, the anti-IL1R1 antibody is administered to the pediatric patient at a dose of 7 mg/kg once every two weeks. In some embodiments, the anti-IL1R1 antibody is administered to the pediatric patient at a dose of 7.5 mg/kg once every two weeks. In some embodiments, the anti-IL1R1 antibody is administered to the pediatric patient at a dose of 8 mg/kg once every two weeks. In some embodiments, the anti-IL1R1 antibody is administered to the pediatric patient at a dose of 8.5 mg/kg once every two weeks. In some embodiments, the anti-IL1R1 antibody is administered to the pediatric patient at a dose of 9 mg/kg once every two weeks. In some embodiments, the anti-IL1R1 antibody is administered to the pediatric patient at a dose of 9.5 mg/kg once every two weeks. In some embodiments, the anti-IL1R1 antibody is administered to the pediatric patient at a dose of 10 mg/kg once every two weeks.

In some embodiments, the anti-IL1R1 antibody is administered to the pediatric patient at a dose of 1 mg/kg once every four weeks. In some embodiments, the anti-IL1R1 antibody is administered to the pediatric patient at a dose of 1.5 mg/kg once every four weeks. In some embodiments, the anti-IL1R1 antibody is administered to the pediatric patient at a dose of 2 mg/kg once every four weeks. In some embodiments, the anti-IL1R1 antibody is administered to the pediatric patient at a dose of 2.5 mg/kg once every four weeks. In some embodiments, the anti-IL1R1 antibody is administered to the pediatric patient at a dose of 3 mg/kg once every four weeks. In some embodiments, the anti-IL1R1 antibody is administered to the pediatric patient at a dose of 3.5 mg/kg once every four weeks. In some embodiments, the anti-IL1R1 antibody is administered to the pediatric patient at a dose of 4 mg/kg once every four weeks. In some embodiments, the anti-IL1R1 antibody is administered to the pediatric patient at a dose of 4.5 mg/kg once every four weeks. In some embodiments, the anti-IL1R1 antibody is administered to the pediatric patient at a dose of 5 mg/kg once every four weeks. In some embodiments, the anti-IL1R1 antibody is administered to the pediatric patient at a dose of 5.5 mg/kg once every four weeks. In some embodiments, the anti-IL1R1 antibody is administered to the pediatric patient at a dose of 6 mg/kg once every four weeks. In some embodiments, the anti-IL1R1 antibody is administered to the pediatric patient at a dose of 6.5 mg/kg once every four weeks. In some embodiments, the anti-IL1R1 antibody is administered to the pediatric patient at a dose of 7 mg/kg once every four weeks. In some embodiments, the anti-IL1R1 antibody is administered to the pediatric patient at a dose of 7.5 mg/kg once every four weeks. In some embodiments, the anti-IL1R1 antibody is administered to the pediatric patient at a dose of 8 mg/kg once every four weeks. In some embodiments, the anti-IL1R1 antibody is administered to the pediatric patient at a dose of 8.5 mg/kg once every four weeks. In some embodiments, the anti-IL1R1 antibody is administered to the pediatric patient at a dose of 9 mg/kg once every four weeks. In some embodiments, the anti-IL1R1 antibody is administered to the pediatric patient at a dose of 9.5 mg/kg once every four weeks. In some embodiments, the anti-IL1R1 antibody is administered to the pediatric patient at a dose of 10 mg/kg once every four weeks.

In some embodiments, the anti-IL1R1 antibody is administered to the pediatric patient at a dose of 10 mg. In some embodiments, the anti-IL1R1 antibody is administered to the pediatric patient at a dose of 20 mg. In some embodiments, the anti-IL1R1 antibody is administered to the pediatric patient at a dose of 30 mg. In some embodiments, the anti-IL1R1 antibody is administered to the pediatric patient at a dose of 40 mg. In some embodiments, the anti-IL1R1 antibody is administered to the pediatric patient at a dose of 50 mg. In some embodiments, the anti-IL1R1 antibody is administered to the pediatric patient at a dose of 60 mg. In some embodiments, the anti-IL1R1 antibody is administered to the pediatric patient at a dose of 70 mg. In some embodiments, the anti-IL1R1 antibody is administered to the pediatric patient at a dose of 80 mg. In some embodiments, the anti-IL1R1 antibody is administered to the pediatric patient at a dose of 90 mg. In some embodiments, the anti-IL1R1 antibody is administered to the pediatric patient at a dose of 100 mg.

In some embodiments, the anti-IL1R1 antibody is administered to the pediatric patient at a dose of 110 mg. In some embodiments, the anti-IL1R1 antibody is administered to the pediatric patient at a dose of 120 mg. In some embodiments, the anti-IL1R1 antibody is administered to the pediatric patient at a dose of 130 mg. In some embodiments, the anti-IL1R1 antibody is administered to the pediatric patient at a dose of 140 mg. In some embodiment, the anti-IL1R1 antibody is administered to the pediatric patient at a dose of 150 mg. In some embodiments, the anti-IL1R1 antibody is administered to the pediatric patient at a dose of 160 mg. In some embodiments, the anti-IL1R1 antibody is administered to the pediatric patient at a dose of 170 mg. In some embodiments, the anti-IL1R1 antibody is administered to the pediatric patient at a dose of 180 mg. In some embodiments, the anti-IL1R1 antibody is administered to the pediatric patient at a dose of 190 mg. In some embodiments, the anti-IL1R1 antibody is administered to the pediatric patient at a dose of 200 mg.

In some embodiments, the anti-IL1R1 antibody is administered to the pediatric patient at a dose of 210 mg. In some embodiments, the anti-IL1R1 antibody is administered to the pediatric patient at a dose of 220 mg. In some embodiments, the anti-IL1R1 antibody is administered to the pediatric patient at a dose of 230 mg. In some embodiments, the anti-IL1R1 antibody is administered to the pediatric patient at a dose of 240 mg. In some embodiment, the anti-IL1R1 antibody is administered to the pediatric patient at a dose of 250 mg. In some embodiments, the anti-IL1R1 antibody is administered to the pediatric patient at a dose of 260 mg. In some embodiments, the anti-IL1R1 antibody is administered to the pediatric patient at a dose of 270 mg. In some embodiments, the anti-IL1R1 antibody is administered to the pediatric patient at a dose of 280 mg. In some embodiments, the anti-IL1R1 antibody is administered to the pediatric patient at a dose of 290 mg. In some embodiments, the anti-IL1R1 antibody is administered to the pediatric patient at a dose of 300 mg.

In some embodiments, the anti-IL1R1 antibody is administered to the pediatric patient at a dose of 10 mg once every two weeks. In some embodiments, the anti-IL1R1 antibody is administered to the pediatric patient at a dose of 20 mg once every two weeks. In some embodiments, the anti-IL1R1 antibody is administered to the pediatric patient at a dose of 30 mg once every two weeks. In some embodiments, the anti-IL1R1 antibody is administered to the pediatric patient at a dose of 40 mg once every two weeks. In some embodiment, the anti-IL1R1 antibody is administered to the pediatric patient at a dose of 50 mg once every two weeks. In some embodiments, the anti-IL1R1 antibody is administered to the pediatric patient at a dose of 60 mg once every two weeks. In some embodiments, the anti-IL1R1 antibody is administered to the pediatric patient at a dose of 70 mg once every two weeks. In some embodiments, the anti-IL1R1 antibody is administered to the pediatric patient at a dose of 80 mg once every two weeks. In some embodiments, the anti-IL1R1 antibody is administered to the pediatric patient at a dose of 90 mg once every two weeks. In some embodiments, the anti-IL1R1 antibody is administered to the pediatric patient at a dose of 100 mg once every two weeks.

In some embodiments, the anti-IL1R1 antibody is administered to the pediatric patient at a dose of 110 mg once every two weeks. In some embodiments, the anti-IL1R1 antibody is administered to the pediatric patient at a dose of 120 mg once every two weeks. In some embodiments, the anti-IL1R1 antibody is administered to the pediatric patient at a dose of 130 mg once every two weeks. In some embodiments, the anti-IL1R1 antibody is administered to the pediatric patient at a dose of 140 mg once every two weeks. In some embodiment, the anti-IL1R1 antibody is administered to the pediatric patient at a dose of 150 mg once every two weeks. In some embodiments, the anti-IL1R1 antibody is administered to the pediatric patient at a dose of 160 mg once every two weeks. In some embodiments, the anti-IL1R1 antibody is administered to the pediatric patient at a dose of 170 mg once every two weeks. In some embodiments, the anti-IL1R1 antibody is administered to the pediatric patient at a dose of 180 mg once every two weeks. In some embodiments, the anti-IL1R1 antibody is administered to the pediatric patient at a dose of 190 mg once every two weeks. In some embodiments, the anti-IL1R1 antibody is administered to the pediatric patient at a dose of 200 mg once every two weeks.

In some embodiments, the anti-IL1R1 antibody is administered to the pediatric patient at a dose of 210 mg once every two weeks. In some embodiments, the anti-IL1R1 antibody is administered to the pediatric patient at a dose of 220 mg once every two weeks. In some embodiments, the anti-IL1R1 antibody is administered to the pediatric patient at a dose of 230 mg once every two weeks. In some embodiments, the anti-IL1R1 antibody is administered to the pediatric patient at a dose of 240 mg once every two weeks. In some embodiment, the anti-IL1R1 antibody is administered to the pediatric patient at a dose of 250 mg once every two weeks. In some embodiments, the anti-IL1R1 antibody is administered to the pediatric patient at a dose of 260 mg once every two weeks. In some embodiments, the anti-IL1R1 antibody is administered to the pediatric patient at a dose of 270 mg once every two weeks. In some embodiments, the anti-IL1R1 antibody is administered to the pediatric patient at a dose of 280 mg once every two weeks. In some embodiments, the anti-IL1R1 antibody is administered to the pediatric patient at a dose of 290 mg once every two weeks. In some embodiments, the anti-IL1R1 antibody is administered to the pediatric patient at a dose of 300 mg once every two weeks.

In some embodiments, the anti-IL1R1 antibody is administered to the pediatric patient at a dose of 10 mg once every four weeks. In some embodiments, the anti-IL1R1 antibody is administered to the pediatric patient at a dose of 20 mg once every four weeks. In some embodiments, the anti-IL1R1 antibody is administered to the pediatric patient at a dose of 30 mg once every four weeks. In some embodiments, the anti-IL1R1 antibody is administered to the pediatric patient at a dose of 40 mg once every four weeks. In some embodiment, the anti-IL1R1 antibody is administered to the pediatric patient at a dose of 50 mg once every four weeks. In some embodiments, the anti-IL1R1 antibody is administered to the pediatric patient at a dose of 60 mg once every four weeks. In some embodiments, the anti-IL1R1 antibody is administered to the pediatric patient at a dose of 70 mg once every four weeks. In some embodiments, the anti-IL1R1 antibody is administered to the pediatric patient at a dose of 80 mg once every four weeks. In some embodiments, the anti-IL1R1 antibody is administered to the pediatric patient at a dose of 90 mg once every four weeks. In some embodiments, the anti-IL1R1 antibody is administered to the pediatric patient at a dose of 100 mg once every four weeks.

In some embodiments, the anti-IL1R1 antibody is administered to the pediatric patient at a dose of 110 mg once every four weeks. In some embodiments, the anti-IL1R1 antibody is administered to the pediatric patient at a dose of 120 mg once every four weeks. In some embodiments, the anti-IL1R1 antibody is administered to the pediatric patient at a dose of 130 mg once every four weeks. In some embodiments, the anti-IL1R1 antibody is administered to the pediatric patient at a dose of 140 mg once every four weeks. In some embodiment, the anti-IL1R1 antibody is administered to the pediatric patient at a dose of 150 mg once every four weeks. In some embodiments, the anti-IL1R1 antibody is administered to the pediatric patient at a dose of 160 mg once every four weeks. In some embodiments, the anti-IL1R1 antibody is administered to the pediatric patient at a dose of 170 mg once every four weeks. In some embodiments, the anti-IL1R1 antibody is administered to the pediatric patient at a dose of 180 mg once every four weeks. In some embodiments, the anti-IL1R1 antibody is administered to the pediatric patient at a dose of 190 mg once every four weeks. In some embodiments, the anti-IL1R1 antibody is administered to the pediatric patient at a dose of 200 mg once every four weeks.

In some embodiments, the anti-IL1R1 antibody is administered to the pediatric patient at a dose of 210 mg once every four weeks. In some embodiments, the anti-IL1R1 antibody is administered to the pediatric patient at a dose of 220 mg once every four weeks. In some embodiments, the anti-IL1R1 antibody is administered to the pediatric patient at a dose of 230 mg once every four weeks. In some embodiments, the anti-IL1R1 antibody is administered to the pediatric patient at a dose of 240 mg once every four weeks. In some embodiment, the anti-IL1R1 antibody is administered to the pediatric patient at a dose of 250 mg once every four weeks. In some embodiments, the anti-IL1R1 antibody is administered to the pediatric patient at a dose of 260 mg once every four weeks. In some embodiments, the anti-IL1R1 antibody is administered to the pediatric patient at a dose of 270 mg once every four weeks. In some embodiments, the anti-IL1R1 antibody is administered to the pediatric patient at a dose of 280 mg once every four weeks. In some embodiments, the anti-IL1R1 antibody is administered to the pediatric patient at a dose of 290 mg once every four weeks. In some embodiments, the anti-IL1R1 antibody is administered to the pediatric patient at a dose of 300 mg once every four weeks.

### Treatment Period

The present invention provides, among other things, a method comprising administering an anti-IL1R1 antibody for a treatment period sufficient to improve, stabilize, prevent or reduce one or more symptoms of recurrent pericarditis.

In some embodiments, an anti-IL1R1 antibody is administered for at least 4 weeks. In some embodiments, an anti-IL1R1 antibody is administered for at least 5 weeks. In some embodiments, an anti-IL1R1 antibody is administered for at least 6 weeks. In some embodiments, an anti-IL1R1 antibody is administered for at least 8 weeks. In some embodiments, an anti-IL1R1 antibody is administered for at least 10 weeks. In some embodiments, an anti-IL1R1 antibody is administered for at least 12 weeks. In some embodiments, an anti-IL1R1 antibody is administered for at least 14 weeks. In some embodiments, an anti-IL1R1 antibody is administered for at least 16 weeks. In some embodiments, an anti-IL1R1 antibody is administered for at least 18 weeks. In some embodiments, an anti-IL1R1 antibody is administered for at least 20 weeks. In some embodiments, an anti-IL1R1 antibody is administered for at least 24 weeks. In some embodiments, an anti-IL1R1 antibody is administered for at least 28 weeks. In some embodiments, an anti-IL1R1 antibody is administered for at least 30 weeks.

In some embodiments, an anti-IL1R1 antibody is administered for at least 1 month. In some embodiments, an anti-IL1R1 antibody is administered for at least 2 months. In some embodiments, an anti-IL1R1 antibody is administered for at least 3 months. In some embodiments, an anti-IL1R1 antibody is administered for at least 4 months. In some embodiments, an anti-IL1R1 antibody is administered for at least 6 months. In some embodiments, an anti-IL1R1 antibody is administered for at least 8 months. In some embodiments, an anti-IL1R1 antibody is administered for at least 10 months. In some embodiments, an anti-IL1R1 antibody is administered for at least 12 months. In some embodiments, an anti-IL1R1 antibody is administered for at least 14 months. In some embodiments, an anti-IL1R1 antibody is administered for at least 16 months. In some embodiments, an anti-IL1R1 antibody is administered for at least 20 months. In some embodiments, an anti-IL1R1 antibody is administered for at least 24 months.

### Exemplary Medical Uses

In one aspect, there is provided an anti-interleukin-1 receptor type-1 (IL1R1) antibody for use in a method of treating recurrent pericarditis (e.g., idiopathic recurrent pericarditis) in a subject in need thereof.

In another aspect, there is provided an anti-interleukin-1 receptor type-1 (IL1R1) antibody for use in method of reducing the risk of a pericarditis recurrence in a subject in need thereof. For instance, the subject may have been previously diagnosed with or treated for recurrent pericarditis (e.g., idiopathic recurrent pericarditis).

In a further aspect, there is provided an anti-interleukin-1 receptor type-1 (IL1R1) antibody for use in method of reducing symptoms of recurrent pericarditis in a subject in need thereof.

In a further aspect, there is provided an anti-interleukin-1 receptor type-1 (IL1R1) antibody for use in a method of treating recurrent pericarditis or reducing the risk of a pericarditis recurrence in a subject in need thereof.

In a further aspect, there is provided an anti-interleukin-1 receptor type-1 (IL1R1) antibody for use in a method of reducing symptoms or risk of recurrent pericarditis in a subject in need thereof.

In a further aspect, there is provided an anti-interleukin-1 receptor type-1 (IL1R1) antibody for use in a method for treatment of idiopathic recurrent pericarditis or reduction in risk of recurrence in a subject in need thereof.

Such uses comprise administering a therapeutically effective amount of the anti-ILR1 antibody (e.g., KPL-387) to the subject to treat the recurrent pericarditis or reduce the risk of recurrence. The therapeutically effective amount may be administered as a single dose or multiple doses and may comprise 50 mg to 400 mg of the anti-IL1R1 antibody. For example, a single dose of 50 mg to 150 mg, or 150 mg to 300 mg, of the anti-IL1R1 antibody may be administered to the subject. In some embodiments, about 50 mg, about 75 mg, about 100 mg, about 125 mg, about 150 mg, about 175 mg, about 200 mg, about 225 mg, about 250 mg, about 275 mg, or about 300 mg may be administered in a single dose. For instance, two single doses of 150 mg may be administered in a single doctor's visit to achieve a total dose of 300 mg per administration interval. A suitable administration interval may be once every two weeks or once every four weeks.

Typically, the anti-IL1R1 antibody is administered for at least four weeks, e.g., for at least eight weeks, at least twelve weeks, at least 16 weeks, at least 20 weeks, or at least 24 weeks. For instance, a four-to-eight-week treatment period may reduce symptoms of recurrent pericarditis (e.g., symptoms of idiopathic recurrent pericarditis) such as C-reactive protein levels and/or pain (e.g., as determined by a numerical rating scale score) to normal or close to normal levels. Continued administration of the IL1R1 antibody for a period of 12 to 24 weeks may prevent recurrence of pericarditis (e.g., idiopathic recurrent pericarditis).

In an exemplary embodiment, a dose of 50-150 mg (e.g., a 100 mg dose) of the anti-IL1R1 antibody is administered to the subject once every two weeks, e.g., for a period of at least four weeks (for instance, for up to 12 weeks or 24 weeks).

In another exemplary embodiment, a dose of 50-150 mg (e.g., a 100 mg dose) of the anti-IL1R1 antibody is administered to the subject once every four weeks, e.g., for a period of at least eight weeks (for instance, for up to 12 weeks or 24 weeks).

In a further exemplary embodiment, a dose of 200-400 mg (e.g., a 300 mg dose) of the anti-IL1R1 antibody is administered to the subject once every two weeks, e.g., for at least four weeks (for instance, for up to 12 weeks or 24 weeks).

In another exemplary embodiment, a dose of 200-400 mg (e.g., a 300 mg dose) of the anti-IL1R1 antibody is administered to the subject once every four weeks, e.g., for at least eight weeks (for instance, for up to 12 weeks or 24 weeks).

The anti-IL1R1 antibody (e.g., KPL-387) may be administered subcutaneously or intravenously. Typically, the anti-IL1R1 antibody is administered subcutaneously, especially when repeated dosing over a longer period (e.g., 12-24 weeks) is required.

A suitable anti-IL1R1 antibody for the medical uses described herein is KPL-387, or another IgG antibody (e.g., an IgG2 antibody) with the same or substantially the same complementarity determining regions (CDRs) as KPL-387 that competes with KPL-387 for binding of IL-1R1. The CDRs of the anti-IL1R1 antibody may vary depending on the numbering system (e.g., Kabat, Chothia or IMGT) used to determine the CDRs.

For example, the anti-IL1R1 antibody may comprise a heavy chain complementarity determining region (HCDR) 1 comprising an amino acid sequence of FHWIA (SEQ ID NO: 1), an HCDR2 of IIHPGASDTRYSPSFQG (SEQ ID NO: 2), and an HCDR3 of QRELDYFDY (SEQ ID NO: 3); and a light chain complementarity determining region (LCDR) 1 comprising an amino acid sequence of RASQSIGSSLH (SEQ ID NO: 4), an LCDR2 of YASQSFS (SEQ ID NO: 5), and an LCDR3 of HQSSSLPLT (SEQ ID NO: 6) as determined using Kabat.

Alternatively, the anti-IL1R1 antibody may comprise an HCDR1 comprising an amino acid sequence of GYSFSFH (SEQ ID NO: 12), an HCDR2 of HPGASD (SEQ ID NO: 13), and an HCDR3 of QRELDYFDY (SEQ ID NO: 3); and an LCDR1 comprising an amino acid sequence of RASQSIGSSLH (SEQ ID NO: 4), an LCDR2 of YASQSFS (SEQ ID NO: 5), and an LCDR3 of HQSSSLPLT (SEQ ID NO: 6) as determined using Chothia.

Alternatively, the anti-IL1R1 antibody may comprise an HCDR1 comprising an amino acid sequence of GYSFSFHW (SEQ ID NO: 14), an HCDR2 of IHPGASDT (SEQ ID NO: 15), and an HCDR3 of ARQRELDYFDY (SEQ ID NO: 19); and an LCDR1 of QSIGSS (SEQ ID NO: 16), an LCDR2 of YAS (SEQ ID NO: 17), and an LCDR3 of HQSSSLPLT (SEQ ID NO: 6) as determined using IMGT.

The anti-IL1R1 antibody may comprise an immunoglobulin heavy chain variable (V_{H}) region comprising an amino acid sequence that is at least about 85% (e.g., at least 90%, 95%, or 99%) identical to, or identical to, the sequence of SEQ ID NO: 7 and an immunoglobulin light chain variable (V_{L}) region comprising an amino acid sequence that is at least about 85% (e.g., at least 90%, 95%, or 99%) identical to, or identical to, the sequence of SEQ ID NO: 8.

For example, the anti-IL1R1 antibody may comprise an immunoglobulin heavy chain variable (V_{H}) region comprising an amino acid sequence that is at least about 97% (e.g., at least 98% or 99%) identical to the sequence of SEQ ID NO: 7 and an immunoglobulin light chain variable (V_{L}) region comprising an amino acid sequence that is at least about 97% (e.g., at least 98% or 99%) identical to the sequence of SEQ ID NO: 8. In some embodiments, the anti-IL1R1 antibody comprises a V_{H} region comprising the amino acid sequence of SEQ ID NO: 7 and a V_{L} region comprising the amino acid sequence of SEQ ID NO: 8. The anti-IL1R1 antibody may comprise an immunoglobulin heavy chain comprising an amino acid sequence that is at least about 85% (e.g., at least 90%, 95%, or 99%) identical to, or identical to, the sequence of SEQ ID NO: 10 and an immunoglobulin light chain comprising an amino acid sequence that is at least about 85% (e.g., at least 90%, 95%, or 99%) identical to, or identical to, the sequence of SEQ ID NO: 11.

For example, the anti-IL1R1 antibody may comprise an immunoglobulin heavy chain comprising an amino acid sequence that is at least about 97% (e.g., at least 98% or 99%) identical to the sequence of SEQ ID NO: 10 and an immunoglobulin light chain comprising an amino acid sequence that is at least about 97% (e.g., at least 98% or 99%) identical to the sequence of SEQ ID NO: 11.

In a specific embodiment, the anti-IL1R1 antibody comprises a heavy chain comprising the amino acid sequence of SEQ ID NO: 10, and a light chain comprising the amino acid sequence of SEQ ID NO: 11.

In an exemplary embodiment, provided is an anti-interleukin-1 receptor type-1 (IL1R1) antibody comprising a heavy chain comprising the amino acid sequence that is at least about 97% (e.g., at least 98% or 99%) identical, or identical, to the sequence of SEQ ID NO: 10, and a light chain comprising the amino acid sequence that is at least about 97% (e.g., at least 98% or 99%) identical to, or identical to, the sequence of SEQ ID NO: 11 for use in a method of treating recurrent pericarditis (e.g., idiopathic recurrent pericarditis) in a subject in need thereof, wherein the anti-IL-1R1 is administered to the subject (e.g., subcutaneously) at a single dose of 50-400 mg (e.g., a 100 mg dose, or 300 mg dose) once every two weeks or once every four weeks.

Administration of the anti-IL1R1 antibody to the subject may improve, stabilize, prevent, or reduce one or more symptoms of recurrent pericarditis (e.g., idiopathic recurrent pericarditis) in the subject relative to a control subject. The control subject may be the subject prior to the administration of the anti-IL1R1 antibody. Alternatively, the control subject may be a comparable recipient to which the anti-IL1R1 antibody has not been administered (e.g., a comparable recipient who was administered a placebo).

### Pharmacokinetics and Pharmacodynamics

Evaluation of an anti-IL1R1 antibody concentration-time profiles in serum of subjects with pericarditis may be evaluated directly by measuring systemic serum anti-IL1R1 antibody concentration-time profiles. Typically, anti-IL1R1 antibody pharmacokinetic and pharmacodynamic profiles are evaluated by sampling the blood of treated subjects periodically. The following standard abbreviations are used to represent the associated pharmacokinetic parameters.
Cₘₐₓ maximum concentration
tₘₐₓ time to maximum concentration
AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity calculated using the formula:

$${AUC}_{0 - ∞} = {AUC}_{0 - t} + \frac{{C}_{t}}{{λ}_{z}}$$

where Cₜ is the last measurable concentration and λ_{Z} is the apparent terminal elimination rate constant
λ_{Z} apparent terminal elimination rate constant, where λ_{Z} is the magnitude of the slope of the linear regression of the log concentration versus time profile during the terminal phase
t_{1/2} apparent terminal elimination half-life (whenever possible),
   where

t_{1/2}=natural log(ln)(2)/λ_{Z}
CL clearance
Vd volume of distribution (IV doses only)
Vd/F apparent volume of distribution (SC doses only)

Typically, actual blood sample collection times relative to the start of anti-IL1R1 antibody administration are used in PK analysis. For example, blood samples are typically collected, for example, within 15 or 30 minutes prior to anti-IL1R1 antibody administration (pre-injection baseline or time 0) and at periodic intervals following administration, e.g., hours 1, 4, 8 or 12, or days 1 (24 hours), 2, 3, 4, 5, 6, 7, 10, 14, 17, 21, 24, 28, 31, 38, 45, 52, 60, 70 or 90 days, following administration. In some embodiments, the blood samples are collected prior to an administration timepoint.

Various methods may be used to measure anti-IL1R1 antibody concentration in serum. As a non-limiting example, enzyme-linked immunosorbent assay (ELISA) methods are used.

Pharmacokinetic parameters may be evaluated at any stage during the treatment, for example, at day 1, day 2, day 3, day 4, day 5, day 6, week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or later. In some embodiments, pharmacokinetic parameters may be evaluated at month 1, month 2, month 3, month 4, month 5, month 6, month 7, month 8, month 9, month 10, month 11, month 12, month 13, month 14, month 15, month 16, month 17, month 18, month 19, month 20, month 21, month 22, month 23, month 24, or later during the treatment.

In one aspect, the present invention provides, among other things, a method of treating recurrent pericarditis comprising administering a patient, an anti-interleukin-1 receptor type-1 (IL1R1) antibody at a therapeutically effective dose that produces a median Cₘₐₓ of at least 0.1 μg/mL. In some embodiments, the therapeutically effective dose produces a median Cₘₐₓ of at least 0.2 μg/mL. In some embodiments, the therapeutically effective dose produces a median Cₘₐₓ of at least 0.3 μg/mL. In some embodiments, the therapeutically effective dose produces a median Cₘₐₓ of at least 0.5 μg/mL. In some embodiments, the therapeutically effective dose produces a median Cₘₐₓ of at least 0.6 μg/mL. In some embodiments, the therapeutically effective dose produces a median Cₘₐₓ of at least 0.8 μg/mL. In some embodiments, the therapeutically effective dose produces a median Cₘₐₓ of at least 0.9 μg/mL. In some embodiments, the therapeutically effective dose produces a median Cₘₐₓ of at least 1.0 μg/mL. In some embodiments, the therapeutically effective dose produces a median Cₘₐₓ of at least 3 μg/mL. In some embodiments, the therapeutically effective dose produces a median Cₘₐₓ of at least 5 μg/mL. In some embodiments, the therapeutically effective dose produces a median Cₘₐₓ of at least 7 μg/mL. In some embodiments, the therapeutically effective dose produces a median Cₘₐₓ of at least 8 μg/mL. In some embodiments, the therapeutically effective dose produces a median Cₘₐₓ of at least 9 μg/mL. In some embodiments, the therapeutically effective dose produces a median Cₘₐₓ of at least 10 μg/mL. In some embodiments, the therapeutically effective dose produces a median Cₘₐₓ of at least 12 μg/mL. In some embodiments, the therapeutically effective dose produces a median Cₘₐₓ of at least 15 μg/mL. In some embodiments, the therapeutically effective dose produces a median Cₘₐₓ of at least 20 μg/mL. In some embodiments, the therapeutically effective dose produces a median Cₘₐₓ of at least 25 μg/mL. In some embodiments, the therapeutically effective dose produces a median Cₘₐₓ of at least 30 μg/mL. In some embodiments, the therapeutically effective dose produces a median Cₘₐₓ of at least 40 μg/mL. In some embodiments, the therapeutically effective dose produces a median Cₘₐₓ of at least 50 μg/mL.

In one aspect, the present invention provides, among other things, a method of treating recurrent pericarditis comprising administering a patient, an anti-interleukin-1 receptor type-1 (IL1R1) antibody at a therapeutically effective dose that maintains mean serum concentration greater than EC₉₀. Typically, EC₉₀ is a concentration of an anti-interleukin-1 receptor type-1 (IL1R1) antibody that results in 90% of the maximal effective activity. In some embodiments, effective activity is inhibition of IL-1-stimulated IL-6 production in primary human cells. In some embodiments, administration of an anti-IL1R1 antibody results in a decrease in IL-6 levels in the subject. In some embodiments, administration of an anti-IL1R1 antibody results in the normalization of IL-6 levels in the subject.

In some embodiments, EC₉₀ is at least 0.01 μg/mL. In some embodiments, EC₉₀ is at least 0.02 μg/mL. In some embodiments, EC₉₀ is at least 0.03 μg/mL. In some embodiments, EC₉₀ is at least 0.04 μg/mL. In some embodiments, EC₉₀ is at least 0.05 μg/mL. In some embodiments, EC₉₀ is at least 0.06 μg/mL. In some embodiments, EC₉₀ is at least 0.07 μg/mL. In some embodiments, EC₉₀ is at least 0.08 μg/mL. In some embodiments, EC₉₀ is at least 0.09 μg/mL. In some embodiments, EC₉₀ is at least 0.1 μg/mL. In some embodiments, EC₉₀ is at least 0.2 μg/mL. In some embodiments, EC₉₀ is at least 0.3 μg/mL. In some embodiments, EC₉₀ is at least 0.4 μg/mL. In some embodiments, EC₉₀ is at least 0.5 μg/mL. In some embodiments, EC₉₀ is at least 0.6 μg/mL. In some embodiments, EC₉₀ is at least 0.7 μg/mL. In some embodiments, EC₉₀ is at least 0.8 μg/mL. In some embodiments, EC₉₀ is at least 0.9 μg/mL. In some embodiments, EC₉₀ is at least 1.0 μg/mL. In some embodiments, EC₉₀ is at least 1.5 μg/mL. In some embodiments, EC₉₀ is at least 2.0 μg/mL. In some embodiments, EC₉₀ is at least 2.5 μg/mL. In some embodiments, EC₉₀ is at least 3 μg/mL. In some embodiments, EC₉₀ is at least 4 μg/mL. In some embodiments, EC₉₀ is at least 5 μg/mL. In some embodiments, EC₉₀ is at least 6 μg/mL. In some embodiments, EC₉₀ is at least 7 μg/mL. In some embodiments, EC₉₀ is at least 8 μg/mL. In some embodiments, EC₉₀ is at least 9 μg/mL. In some embodiments, EC₉₀ is at least 10 μg/mL. In some embodiments, EC₉₀ is at least 12 μg/mL. In some embodiments, EC₉₀ is at least 15 μg/mL. In some embodiments, EC₉₀ is at least 18 μg/mL. In some embodiments, EC₉₀ is at least 20 μg/mL. In some embodiments, EC₉₀ is at least 22 μg/mL. In some embodiments, EC₉₀ is at least 25 μg/mL. In some embodiments, EC₉₀ is at least 28 μg/mL. In some embodiments, EC₉₀ is at least 30 μg/mL. In some embodiments, EC₉₀ is at least 32 μg/mL. In some embodiments, EC₉₀ is at least 35 μg/mL. In some embodiments, EC₉₀ is at least 38 μg/mL. In some embodiments, EC₉₀ is at least 40 μg/mL. In some embodiments, EC₉₀ is at least 42 μg/mL. In some embodiments, EC₉₀ is at least 45 μg/mL. In some embodiments, EC₉₀ is at least 48 μg/mL. In some embodiments, EC₉₀ is at least 50 μg/mL.

In some embodiments, EC₉₀ is between 0.01 and 50 μg/mL. In some embodiments, EC₉₀ is between 0.02 and 40 μg/mL. In some embodiments, EC₉₀ is between 0.03 and 40 μg/mL. In some embodiments, EC₉₀ is between 0.03 and 30 μg/mL. In some embodiments, EC₉₀ is between 0.05 and 25 μg/mL. In some embodiments, EC₉₀ is between 0.05 and 20 μg/mL. In some embodiments, EC₉₀ is between 0.1 and 20 μg/mL. In some embodiments, EC₉₀ is between 0.1 and 15 μg/mL. In some embodiments, EC₉₀ is between 0.1 and 10 μg/mL. In some embodiments, EC₉₀ is between 0.3 and 10 μg/mL. In some embodiments, EC₉₀ is between 0.4 and 8 μg/mL. In some embodiments, EC₉₀ is between 0.5 and 5 μg/mL. In some embodiments, EC₉₀ is between 0.1 and 5 μg/mL. In some embodiments, EC₉₀ is between 0.1 and 3 μg/mL. In some embodiments, EC₉₀ is between 0.3 and 3 μg/mL. In some embodiments, EC₉₀ is between 0.5 and 1 μg/mL.

In some embodiments, EC₉₀ is 0.01 μg/mL. In some embodiments, EC₉₀ is 0.02 μg/mL. In some embodiments, EC₉₀ is 0.03 μg/mL. In some embodiments, EC₉₀ is 0.04 μg/mL. In some embodiments, EC₉₀ is 0.05 μg/mL. In some embodiments, EC₉₀ is 0.06 μg/mL. In some embodiments, EC₉₀ is 0.07 μg/mL. In some embodiments, EC₉₀ is 0.08 μg/mL. In some embodiments, EC₉₀ is 0.09 μg/mL. In some embodiments, EC₉₀ is 0.1 μg/mL. In some embodiments, EC₉₀ is 0.2 μg/mL. In some embodiments, EC₉₀ is 0.3 μg/mL. In some embodiments, EC₉₀ is 0.4 μg/mL. In some embodiments, EC₉₀ is 0.5 μg/mL. In some embodiments, EC₉₀ is 0.6 μg/mL. In some embodiments, EC₉₀ is 0.7 μg/mL. In some embodiments, EC₉₀ is 0.8 μg/mL. In some embodiments, EC₉₀ is 0.9 μg/mL. In some embodiments, EC₉₀ is 1.0 μg/mL. In some embodiments, EC₉₀ is 1.5 μg/mL. In some embodiments, EC₉₀ is 2.0 μg/mL. In some embodiments, EC₉₀ is 2.5 μg/mL. In some embodiments, EC₉₀ is 3 μg/mL. In some embodiments, EC₉₀ is 4 μg/mL. In some embodiments, EC₉₀ is 5 μg/mL. In some embodiments, EC₉₀ is 6 μg/mL. In some embodiments, EC₉₀ is 7 μg/mL. In some embodiments, EC₉₀ is 8 μg/mL. In some embodiments, EC₉₀ is 9 μg/mL. In some embodiments, EC₉₀ is 10 μg/mL. In some embodiments, EC₉₀ is 12 μg/mL. In some embodiments, EC₉₀ is 15 μg/mL. In some embodiments, EC₉₀ is 18 μg/mL. In some embodiments, EC₉₀ is 20 μg/mL. In some embodiments, EC₉₀ is 22 μg/mL. In some embodiments, EC₉₀ is 25 μg/mL. In some embodiments, EC₉₀ is 28 μg/mL. In some embodiments, EC₉₀ is 30 μg/mL. In some embodiments, EC₉₀ is 32 μg/mL. In some embodiments, EC₉₀ is 35 μg/mL. In some embodiments, EC₉₀ is 38 μg/mL. In some embodiments, EC₉₀ is 40 μg/mL. In some embodiments, EC₉₀ is 42 μg/mL. In some embodiments, EC₉₀ is 45 μg/mL. In some embodiments, EC₉₀ is 48 μg/mL. In some embodiments, EC₉₀ is 50 μg/mL.

In some embodiments, the therapeutically effective dose maintains mean serum concentration greater than EC₉₀ for at least 3 days. In some embodiments, the therapeutically effective dose maintains mean serum concentration greater than EC₉₀ for at least 5 days. In some embodiments, the therapeutically effective dose maintains mean serum concentration greater than EC₉₀ for at least 7 days. In some embodiments, the therapeutically effective dose maintains mean serum concentration greater than EC₉₀ for at least 10 days. In some embodiments, the therapeutically effective dose maintains mean serum concentration greater than EC₉₀ for at least 12 days. In some embodiments, the therapeutically effective dose maintains mean serum concentration greater than EC₉₀ for at least 14 days. In some embodiments, the therapeutically effective dose maintains mean serum concentration greater than EC₉₀ for at least 15 days. In some embodiments, the therapeutically effective dose maintains mean serum concentration greater than EC₉₀ for at least 18 days. In some embodiments, the therapeutically effective dose maintains mean serum concentration greater than EC₉₀ for at least 20 days. In some embodiments, the therapeutically effective dose maintains mean serum concentration greater than EC₉₀ for at least 21 days. In some embodiments, the therapeutically effective dose maintains mean serum concentration greater than EC₉₀ for at least 25 days. In some embodiments, the therapeutically effective dose maintains mean serum concentration greater than EC₉₀ for at least 28 days. In some embodiments, the therapeutically effective dose maintains mean serum concentration greater than EC₉₀ for at least 30 days. In some embodiments, the therapeutically effective dose maintains mean serum concentration greater than EC₉₀ for at least 35 days. In some embodiments, the therapeutically effective dose maintains mean serum concentration greater than EC₉₀ for at least 40 days. In some embodiments, the therapeutically effective dose maintains mean serum concentration greater than EC₉₀ for at least 42 days. In some embodiments, the therapeutically effective dose maintains mean serum concentration greater than EC₉₀ for at least 45 days. In some embodiments, the therapeutically effective dose maintains mean serum concentration greater than EC₉₀ for at least 50 days. In some embodiments, the therapeutically effective dose maintains mean serum concentration greater than EC₉₀ for at least 56 days. In some embodiments, the therapeutically effective dose maintains mean serum concentration greater than EC₉₀ for at least 60 days.

In some embodiments, the therapeutically effective dose maintains mean serum concentration greater than EC₉₀ for at least 1 week. In some embodiments, the therapeutically effective dose maintains mean serum concentration greater than EC₉₀ for at least 2 weeks. In some embodiments, the therapeutically effective dose maintains mean serum concentration greater than EC₉₀ for at least 3 weeks. In some embodiments, the therapeutically effective dose maintains mean serum concentration greater than EC₉₀ for at least 4 weeks. In some embodiments, the therapeutically effective dose maintains mean serum concentration greater than EC₉₀ for at least 5 weeks. In some embodiments, the therapeutically effective dose maintains mean serum concentration greater than EC₉₀ for at least 6 weeks. In some embodiments, the therapeutically effective dose maintains mean serum concentration greater than EC₉₀ for at least 7 weeks.

### Clinical Efficacy Metrics/Endpoints

### Numerical Rating Scale (NRS)

In some embodiments, the effectiveness of the treatment using an anti-IL1R1 antibody is determined by assessment of pain in the subject, such as by determination of 0-10 point NRS score.

In some embodiments, the patient has at least 1 day with pericarditis pain measurement 10 on NRS prior to the treatment. In some embodiments, the patient has at least 1 day with pericarditis pain measurement 9 on NRS prior to the treatment. In some embodiments, the patient has at least 1 day with pericarditis pain measurement 8 on NRS prior to the treatment. In some embodiments, the patient has at least 1 day with pericarditis pain measurement 7 on NRS prior to the treatment. In some embodiments, the patient has at least 1 day with pericarditis pain measurement 6 on NRS prior to the treatment. In some embodiments, the patient has at least 1 day with pericarditis pain measurement 5 on NRS prior to the treatment. In some embodiments, the patient has at least 1 day with pericarditis pain measurement ≥4 on NRS prior to the treatment. In some embodiments, the patient has at least 1 day with pericarditis pain measurement ≥3 on NRS prior to the treatment. In some embodiments, the patient has at least 1 day with pericarditis pain measurement 2 on NRS prior to the treatment.

In some embodiments, the administration of the anti-IL1R1 antibody results in NRS score of 8 or less. In some embodiments, the administration of the anti-IL1R1 antibody results in NRS score of 7 or less. In some embodiments, the administration of the anti-IL1R1 antibody results in NRS score of 6 or less. In some embodiments, the administration of the anti-IL1R1 antibody results in NRS score of 5 or less. In some embodiments, the administration of the anti-IL1R1 antibody results in NRS score of 4 or less. In some embodiments, the administration of the anti-IL1R1 antibody results in NRS score of 3 or less. In some embodiments, the administration of the anti-IL1R1 antibody results in NRS score of 2 or less. In some embodiments, the administration of the anti-IL1R1 antibody results in NRS score of 1 or less. In some embodiments, the administration of the anti-IL1R1 antibody results in NRS score of 0.

In some embodiments, NRS score of 4 or less is achieved within 12 weeks from the first administration of the anti-IL1R1 antibody. In some embodiments, NRS score of 4 or less is achieved within 10 weeks from the first administration of the anti-IL1R1 antibody. In some embodiments, NRS score of 4 or less is achieved within 8 weeks from the first administration of the anti-IL1R1 antibody. In some embodiments, NRS score of 4 or less is achieved within 7 weeks from the first administration of the anti-IL1R1 antibody. In some embodiments, NRS score of 4 or less is achieved within 6 weeks from the first administration of the anti-IL1R1 antibody. In some embodiments, NRS score of 4 or less is achieved within 5 weeks from the first administration of the anti-IL1R1 antibody. In some embodiments, NRS score of 4 or less is achieved within 4 weeks from the first administration of the anti-IL1R1 antibody. In some embodiments, NRS score of 4 or less is achieved within 3 weeks from the first administration of the anti-IL1R1 antibody. In some embodiments, NRS score of 4 or less is achieved within 2 weeks from the first administration of the anti-IL1R1 antibody. In some embodiments, NRS score of 4 or less is achieved within 1 week from the first administration of the anti-IL1R1 antibody. In some embodiments, NRS score of 4 or less is achieved within 7 days from the first administration of the anti-IL1R1 antibody. In some embodiments, NRS score of 4 or less is achieved within 5 days from the first administration of the anti-IL1R1 antibody. In some embodiments, NRS score of 4 or less is achieved within 3 days from the first administration of the anti-IL1R1 antibody. In some embodiments, NRS score of 4 or less is achieved within 2 days from the first administration of the anti-IL1R1 antibody. In some embodiments, NRS score of 4 or less is achieved within 1 day from the first administration of the anti-IL1R1 antibody. In the foregoing embodiments, the NRS score is determined while the patient continues to receive a therapeutic dose of anti-IL1R1 antibody at an administration interval and treatment period according to the invention.

In some embodiments, NRS score of 2 or less is achieved within 12 weeks from the first administration of the anti-IL1R1 antibody. In some embodiments, NRS score of 2 or less is achieved within 10 weeks from the first administration of the anti-IL1R1 antibody. In some embodiments, NRS score of 2 or less is achieved within 8 weeks from the first administration of the anti-IL1R1 antibody. In some embodiments, NRS score of 2 or less is achieved within 7 weeks from the first administration of the anti-IL1R1 antibody. In some embodiments, NRS score of 2 or less is achieved within 6 weeks from the first administration of the anti-IL1R1 antibody. In some embodiments, NRS score of 2 or less is achieved within 5 weeks from the first administration of the anti-IL1R1 antibody. In some embodiments, NRS score of 2 or less is achieved within 4 weeks from the first administration of the anti-IL1R1 antibody. In some embodiments, NRS score of 2 or less is achieved within 3 weeks from the first administration of the anti-IL1R1 antibody. In some embodiments, NRS score of 2 or less is achieved within 2 weeks from the first administration of the anti-IL1R1 antibody. In some embodiments, NRS score of 2 or less is achieved within 1 week from the first administration of the anti-IL1R1 antibody. In some embodiments, NRS score of 2 or less is achieved within 7 days from the first administration of the anti-IL1R1 antibody. In some embodiments, NRS score of 2 or less is achieved within 5 days from the first administration of the anti-IL1R1 antibody. In some embodiments, NRS score of 2 or less is achieved within 3 days from the first administration of the anti-IL1R1 antibody. In some embodiments, NRS score of 2 or less is achieved within 2 days from the first administration of the anti-IL1R1 antibody. In some embodiments, NRS score of 2 or less is achieved within 1 day from the first administration of the anti-IL1R1 antibody. In the foregoing embodiments, the NRS score is determined while the patient continues to receive a therapeutic dose of anti-IL1R1 antibody at an administration interval and treatment period according to the invention.

In some embodiments, NRS score of 1 or less is achieved within 12 weeks from the first administration of the anti-IL1R1 antibody. In some embodiments, NRS score of 1 or less is achieved within 10 weeks from the first administration of the anti-IL1R1 antibody. In some embodiments, NRS score of 1 or less is achieved within 8 weeks from the first administration of the anti-IL1R1 antibody. In some embodiments, NRS score of 1 or less is achieved within 7 weeks from the first administration of the anti-IL1R1 antibody. In some embodiments, NRS score of 1 or less is achieved within 6 weeks from the first administration of the anti-IL1R1 antibody. In some embodiments, NRS score of 1 or less is achieved within 5 weeks from the first administration of the anti-IL1R1 antibody. In some embodiments, NRS score of 1 or less is achieved within 4 weeks from the first administration of the anti-IL1R1 antibody. In some embodiments, NRS score of 1 or less is achieved within 3 weeks from the first administration of the anti-IL1R1 antibody. In some embodiments, NRS score of 1 or less is achieved within 2 weeks from the first administration of the anti-IL1R1 antibody. In some embodiments, NRS score of 1 or less is achieved within 1 week from the first administration of the anti-IL1R1 antibody. In some embodiments, NRS score of 1 or less is achieved within 7 days from the first administration of the anti-IL1R1 antibody. In some embodiments, NRS score of 1 or less is achieved within 5 days from the first administration of the anti-IL1R1 antibody. In some embodiments, NRS score of 1 or less is achieved within 3 days from the first administration of the anti-IL1R1 antibody. In some embodiments, NRS score of 1 or less is achieved within 2 days from the first administration of the anti-IL1R1 antibody. In some embodiments, NRS score of 1 or less is achieved within 1 day from the first administration of the anti-IL1R1 antibody. In the foregoing embodiments, the NRS score is determined while the patient continues to receive a therapeutic dose of anti-IL1R1 antibody at an administration interval and treatment period according to the invention.

In some embodiments, the NRS score is maintained at 4 or less for longer than 1 week. In some embodiments, the NRS score is maintained at 4 or less for longer than 2 weeks. In some embodiments, the NRS score is maintained at 4 or less for longer than 3 weeks. In some embodiments, the NRS score is maintained at 4 or less for longer than 4 weeks. In some embodiments, the NRS score is maintained at 4 or less for longer than 5 weeks. In some embodiments, the NRS score is maintained at 4 or less for longer than 6 weeks. In some embodiments, the NRS score is maintained at 4 or less for longer than 8 weeks. In some embodiments, the NRS score is maintained at 4 or less for longer than 10 weeks. In some embodiments, the NRS score is maintained at 4 or less for longer than 12 weeks. In some embodiments, the NRS score is maintained at 4 or less for longer than 1 month. In some embodiments, the NRS score is maintained at 4 or less for longer than 2 months. In some embodiments, the NRS score is maintained at 4 or less for longer than 3 months. In some embodiments, the NRS score is maintained at 4 or less for longer than 4 months. In some embodiments, the NRS score is maintained at 4 or less for longer than 5 months. In some embodiments, the NRS score is maintained at 4 or less for longer than 6 months. In some embodiments, the NRS score is maintained at 4 or less for longer than 7 months. In some embodiments, the NRS score is maintained at 4 or less for longer than 8 months. In some embodiments, the NRS score is maintained at 4 or less for longer than 9 months. In some embodiments, the NRS score is maintained at 4 or less for longer than 10 months. In some embodiments, the NRS score is maintained at 4 or less for longer than 11 months. In some embodiments, the NRS score is maintained at 4 or less for longer than 12 months. In some embodiments, the NRS score is maintained at 4 or less for longer than 1 year. In some embodiments, the NRS score is maintained at 4 or less for the above-indicated periods while the patient receives anti-IL1R1 antibody in absence of any concurrent therapy. In the foregoing embodiments, the NRS score is determined while the patient continues to receive a therapeutic dose of anti-IL1R1 antibody at an administration interval and treatment period according to the invention.

In some embodiments, the NRS score is maintained at 2 or less for longer than 1 week. In some embodiments, the NRS score is maintained at 2 or less for longer than 2 weeks. In some embodiments, the NRS score is maintained at 2 or less for longer than 3 weeks. In some embodiments, the NRS score is maintained at 2 or less for longer than 4 weeks. In some embodiments, the NRS score is maintained at 2 or less for longer than 5 weeks. In some embodiments, the NRS score is maintained at 2 or less for longer than 6 weeks. In some embodiments, the NRS score is maintained at 2 or less for longer than 8 weeks. In some embodiments, the NRS score is maintained at 2 or less for longer than 10 weeks. In some embodiments, the NRS score is maintained at 2 or less for longer than 12 weeks. In some embodiments, the NRS score is maintained at 2 or less for longer than 1 month. In some embodiments, the NRS score is maintained at 2 or less for longer than 2 months. In some embodiments, the NRS score is maintained at 2 or less for longer than 3 months. In some embodiments, the NRS score is maintained at 2 or less for longer than 4 months. In some embodiments, the NRS score is maintained at 2 or less for longer than 5 months. In some embodiments, the NRS score is maintained at 2 or less for longer than 6 months. In some embodiments, the NRS score is maintained at 2 or less for longer than 7 months. In some embodiments, the NRS score is maintained at 2 or less for longer than 8 months. In some embodiments, the NRS score is maintained at 2 or less for longer than 9 months. In some embodiments, the NRS score is maintained at 2 or less for longer than 10 months. In some embodiments, the NRS score is maintained at 2 or less for longer than 11 months. In some embodiments, the NRS score is maintained at 2 or less for longer than 12 months. In some embodiments, the NRS score is maintained at 2 or less for longer than 1 year. In some embodiments, the NRS score is maintained at 2 or less for the above-indicated periods while the patient receives anti-IL1R1 antibody in absence of any concurrent therapy. In the foregoing embodiments, the NRS score is determined while the patient continues to receive a therapeutic dose of anti-IL1R1 antibody at an administration interval and treatment period according to the invention.

In some embodiments, the NRS score is maintained at 1 or less for longer than 1 week. In some embodiments, the NRS score is maintained at 1 or less for longer than 2 weeks. In some embodiments, the NRS score is maintained at 1 or less for longer than 3 weeks. In some embodiments, the NRS score is maintained at 1 or less for longer than 4 weeks. In some embodiments, the NRS score is maintained at 1 or less for longer than 5 weeks. In some embodiments, the NRS score is maintained at 1 or less for longer than 6 weeks. In some embodiments, the NRS score is maintained at 1 or less for longer than 8 weeks. In some embodiments, the NRS score is maintained at 1 or less for longer than 10 weeks. In some embodiments, the NRS score is maintained at 1 or less for longer than 12 weeks. In some embodiments, the NRS score is maintained at 1 or less for longer than 1 month. In some embodiments, the NRS score is maintained at 1 or less for longer than 2 months. In some embodiments, the NRS score is maintained at 1 or less for longer than 3 months. In some embodiments, the NRS score is maintained at 1 or less for longer than 4 months. In some embodiments, the NRS score is maintained at 1 or less for longer than 5 months. In some embodiments, the NRS score is maintained at 1 or less for longer than 6 months. In some embodiments, the NRS score is maintained at 1 or less for longer than 7 months. In some embodiments, the NRS score is maintained at 1 or less for longer than 8 months. In some embodiments, the NRS score is maintained at 1 or less for longer than 9 months. In some embodiments, the NRS score is maintained at 1 or less for longer than 10 months. In some embodiments, the NRS score is maintained at 1 or less for longer than 11 months. In some embodiments, the NRS score is maintained at 1 or less for longer than 12 months. In some embodiments, the NRS score is maintained at 1 or less for longer than 1 year. In some embodiments, the NRS score is maintained at 1 or less for the above-indicated periods while the patient receives anti-IL1R1 antibody in absence of any concurrent therapy. In the foregoing embodiments, the NRS score is determined while the patient continues to receive a therapeutic dose of anti-IL1R1 antibody at an administration interval and treatment period according to the invention.

In some embodiments, the patient remains pain free for the duration of the extension period of the study. In some embodiments, the NRS score is maintained at 2 or less for while the patient continues to receive a therapeutic dose of anti-IL1R1 antibody at an administration interval and treatment period according to the invention. In some embodiments, the NRS score is maintained at 2 or less for while the patient continues to receive a therapeutic dose of anti-IL1R1 antibody in absence of any concurrent therapy.

### C-Reactive Protein (CRP)

C-Reactive Protein (CRP) is a recognized indicator of inflammation in pericarditis and recurrent pericarditis. In some embodiments, the effectiveness of the treatment using an anti-IL1R1 antibody is determined by measuring inflammation, such as measuring the CRP level in peripheral blood.

In some embodiments, the patient has C-reactive protein (CRP) value >0.2 mg/dL prior to the treatment. In some embodiments, the patient has C-reactive protein (CRP) value >0.5 mg/dL prior to the treatment. In some embodiments, the patient has C-reactive protein (CRP) value >0.8 mg/dL prior to the treatment. In some embodiments, the patient has C-reactive protein (CRP) value >1 mg/dL prior to the treatment. In some embodiments, the patient has C-reactive protein (CRP) value >2 mg/dL prior to the treatment. In some embodiments, the patient has C-reactive protein (CRP) value >3 mg/dL prior to the treatment. In some embodiments, the patient has C-reactive protein (CRP) value >4 mg/dL prior to the treatment. In some embodiments, the patient has C-reactive protein (CRP) value >5 mg/dL prior to the treatment. In some embodiments, the patient has C-reactive protein (CRP) value >6 mg/dL prior to the treatment. In some embodiments, the patient has C-reactive protein (CRP) value >8 mg/dL prior to the treatment. In some embodiments, the patient has C-reactive protein (CRP) value >10 mg/dL prior to the treatment.

In some embodiments, administration of the anti-IL1R1 antibody results in a reduced CRP value. In some embodiments, administration of the anti-IL1R1 antibody results in a reduced CRP value of ≤5.0 mg/dL. In some embodiments, administration of the anti-IL1R1 antibody results in a reduced CRP value of 4.0 mg/dL. In some embodiments, administration of the anti-IL1R1 antibody results in a reduced CRP value of 3.0 mg/dL. In some embodiments, administration of the anti-IL1R1 antibody results in a reduced CRP value of 2.0 mg/dL. In some embodiments, administration of the anti-IL1R1 antibody results in a reduced CRP value of 1.5 mg/dL. In some embodiments, administration of the anti-IL1R1 antibody results in a reduced CRP value of ≤1.0 mg/dL. In some embodiments, administration of the anti-IL1R1 antibody results in a reduced CRP value of 0.8 mg/dL. In some embodiments, administration of the anti-IL1R1 antibody results in a reduced CRP value of 0.6 mg/dL. In some embodiments, administration of the anti-IL1R1 antibody results in a reduced CRP value of 0.5 mg/dL. In some embodiments, administration of the anti-IL1R1 antibody results in a reduced CRP value of ≤0.4 mg/dL. In some embodiments, administration of the anti-IL1R1 antibody results in a reduced CRP value of 0.3 mg/dL. In some embodiments, administration of the anti-IL1R1 antibody results in a reduced CRP value of 0.2 mg/dL. In some embodiments, administration of the anti-IL1R1 antibody results in a reduced CRP value of 0.1 mg/dL. In some embodiments, administration of the anti-IL1R1 antibody results in a reduced CRP value of substantially 0.

In some embodiments, the CRP value ≤0.5 mg/dL is achieved within 12 weeks from the first administration of the anti-IL1R1 antibody. In some embodiments, the CRP value ≤0.5 mg/dL is achieved within 10 weeks from the first administration of the anti-IL1R1 antibody. In some embodiments, the CRP value ≤0.5 mg/dL is achieved within 8 weeks from the first administration of the anti-IL1R1 antibody. In some embodiments, the CRP value ≤0.5 mg/dL is achieved within 6 weeks from the first administration of the anti-IL1R1 antibody. In some embodiments, the CRP value ≤0.5 mg/dL is achieved within 5 weeks from the first administration of the anti-IL1R1 antibody. In some embodiments, the CRP value ≤0.5 mg/dL is achieved within 4 weeks from the first administration of the anti-IL1R1 antibody. In some embodiments, the CRP value ≤0.5 mg/dL is achieved within 3 weeks from the first administration of the anti-IL1R1 antibody. In some embodiments, the CRP value ≤0.5 mg/dL is achieved within 2 weeks from the first administration of the anti-IL1R1 antibody. In some embodiments, the CRP value ≤0.5 mg/dL is achieved within 1 week from the first administration of the anti-IL1R1 antibody. In some embodiments, the CRP value ≤0.5 mg/dL is achieved within 10 days from the first administration of the anti-IL1R1 antibody. In some embodiments, the CRP value ≤0.5 mg/dL is achieved within 7 days from the first administration of the anti-IL1R1 antibody. In some embodiments, the CRP value ≤0.5 mg/dL is achieved within 5 days from the first administration of the anti-IL1R1 antibody. In some embodiments, the CRP value ≤0.5 mg/dL is achieved within 3 days from the first administration of the anti-IL1R1 antibody. In some embodiments, the CRP value ≤0.5 mg/dL is achieved within 2 days from the first administration of the anti-IL1R1 antibody. In some embodiments, the CRP value ≤0.5 mg/dL is achieved within 1 day from the first administration of the anti-IL1R1 antibody. In the foregoing embodiments, the CRP value is determined while the patient continues to receive a therapeutic dose of anti-IL1R1 antibody at an administration interval and treatment period according to the invention.

In some embodiments, the CRP value ≤1.0 mg/dL is achieved within 12 weeks from the first administration of the anti-IL1R1 antibody. In some embodiments, the CRP value ≤1.0 mg/dL is achieved within 10 weeks from the first administration of the anti-IL1R1 antibody. In some embodiments, the CRP value ≤1.0 mg/dL is achieved within 8 weeks from the first administration of the anti-IL1R1 antibody. In some embodiments, the CRP value ≤1.0 mg/dL is achieved within 6 weeks from the first administration of the anti-IL1R1 antibody. In some embodiments, the CRP value 1.0 mg/dL is achieved within 5 weeks from the first administration of the anti-IL1R1 antibody. In some embodiments, the CRP value ≤1.0 mg/dL is achieved within 4 weeks from the first administration of the anti-IL1R1 antibody. In some embodiments, the CRP value ≤1.0 mg/dL is achieved within 3 weeks from the first administration of the anti-IL1R1 antibody. In some embodiments, the CRP value ≤1.0 mg/dL is achieved within 2 weeks from the first administration of the anti-IL1R1 antibody. In some embodiments, the CRP value ≤1.0 mg/dL is achieved within 1 week from the first administration of the anti-IL1R1 antibody. In some embodiments, the CRP value ≤1.0 mg/dL is achieved within 10 days from the first administration of the anti-IL1R1 antibody. In some embodiments, the CRP value ≤1.0 mg/dL is achieved within 7 days from the first administration of the anti-IL1R1 antibody. In some embodiments, the CRP value ≤1.0 mg/dL is achieved within 5 days from the first administration of the anti-IL1R1 antibody. In some embodiments, the CRP value ≤1.0 mg/dL is achieved within 3 days from the first administration of the anti-IL1R1 antibody. In some embodiments, the CRP value ≤1.0 mg/dL is achieved within 2 days from the first administration of the anti-IL1R1 antibody. In some embodiments, the CRP value ≤1.0 mg/dL is achieved within 1 day from the first administration of the anti-IL1R1 antibody. In the foregoing embodiments, the CRP value is determined while the patient continues to receive a therapeutic dose of anti-IL1R1 antibody at an administration interval and treatment period according to the invention.

In some embodiments, the CRP value ≤2.0 mg/dL is achieved within 12 weeks from the first administration of the anti-IL1R1 antibody. In some embodiments, the CRP value ≤2.0 mg/dL is achieved within 10 weeks from the first administration of the anti-IL1R1 antibody. In some embodiments, the CRP value ≤2.0 mg/dL is achieved within 8 weeks from the first administration of the anti-IL1R1 antibody. In some embodiments, the CRP value ≤2.0 mg/dL is achieved within 6 weeks from the first administration of the anti-IL1R1 antibody. In some embodiments, the CRP value ≤2.0 mg/dL is achieved within 5 weeks from the first administration of the anti-IL1R1 antibody. In some embodiments, the CRP value ≤2.0 mg/dL is achieved within 4 weeks from the first administration of the anti-IL1R1 antibody. In some embodiments, the CRP value ≤2.0 mg/dL is achieved within 3 weeks from the first administration of the anti-IL1R1 antibody. In some embodiments, the CRP value ≤2.0 mg/dL is achieved within 2 weeks from the first administration of the anti-IL1R1 antibody. In some embodiments, the CRP value ≤2.0 mg/dL is achieved within 1 week from the first administration of the anti-IL1R1 antibody. In some embodiments, the CRP value ≤2.0 mg/dL is achieved within 10 days from the first administration of the anti-IL1R1 antibody. In some embodiments, the CRP value ≤2.0 mg/dL is achieved within 7 days from the first administration of the anti-IL1R1 antibody. In some embodiments, the CRP value ≤2.0 mg/dL is achieved within 6 days from the first administration of the anti-IL1R1 antibody. In some embodiments, the CRP value ≤2.0 mg/dL is achieved within 5 days from the first administration of the anti-IL1R1 antibody. In some embodiments, the CRP value ≤2.0 mg/dL is achieved within 4 days from the first administration of the anti-IL1R1 antibody. In some embodiments, the CRP value ≤2.0 mg/dL is achieved within 3 days from the first administration of the anti-IL1R1 antibody. In some embodiments, the CRP value ≤2.0 mg/dL is achieved within 2 days from the first administration of the anti-IL1R1 antibody. In some embodiments, the CRP value ≤2.0 mg/dL is achieved within 1 day from the first administration of the anti-IL1R1 antibody. In the foregoing embodiments, the CRP value is determined while the patient continues to receive a therapeutic dose of anti-IL1R1 antibody at an administration interval and treatment period according to the invention.

In some embodiments, the CRP value is maintained at ≤2.0 mg/dL for longer than 2 weeks. In some embodiments, the CRP value is maintained at ≤2.0 mg/dL for longer than 3 weeks. In some embodiments, the CRP value is maintained at ≤2.0 mg/dL for longer than 4 weeks. In some embodiments, the CRP value is maintained at ≤2.0 mg/dL for longer than 5 weeks. In some embodiments, the CRP value is maintained at ≤2.0 mg/dL for longer than 6 weeks. In some embodiments, the CRP value is maintained at ≤2.0 mg/dL for longer than 8 weeks. In some embodiments, the CRP value is maintained at ≤2.0 mg/dL for longer than 10 weeks. In some embodiments, the CRP value is maintained at ≤2.0 mg/dL for longer than 12 weeks. In some embodiments, the CRP value is maintained at ≤2.0 mg/dL for longer than 1 month. In some embodiments, the CRP value is maintained at ≤2.0 mg/dL for longer than 2 months. In some embodiments, the CRP value is maintained at ≤2.0 mg/dL for longer than 3 months. In some embodiments, the CRP value is maintained at ≤2.0 mg/dL for longer than 4 months. In some embodiments, the CRP value is maintained at ≤2.0 mg/dL for longer than 5 months. In some embodiments, the CRP value is maintained at ≤2.0 mg/dL for longer than 6 months. In some embodiments, the CRP value is maintained at ≤2.0 mg/dL for longer than 7 months. In some embodiments, the CRP value is maintained at ≤2.0 mg/dL for longer than 8 months. In some embodiments, the CRP value is maintained at ≤2.0 mg/dL for longer than 9 months. In some embodiments, the CRP value is maintained at ≤2.0 mg/dL for longer than 10 months. In some embodiments, the CRP value is maintained at ≤2.0 mg/dL for longer than 11 months. In some embodiments, the CRP value is maintained at ≤2.0 mg/dL for longer than 12 months. In some embodiments, the CRP value is maintained at ≤2.0 mg/dL for longer than 1 year. In some embodiments, the CRP level is maintained at ≤2.0 mg/dL for the above-indicated periods while the patient receives anti-IL1R1 antibody in absence of any concurrent therapy. In the foregoing embodiments, the CRP value is determined while the patient continues to receive a therapeutic dose of anti-IL1R1 antibody at an administration interval and treatment period according to the invention.

In some embodiments, the CRP value is maintained at ≤1.0 mg/dL for longer than 2 weeks. In some embodiments, the CRP value is maintained at ≤1.0 mg/dL for longer than 3 weeks. In some embodiments, the CRP value is maintained at ≤1.0 mg/dL for longer than 4 weeks. In some embodiments, the CRP value is maintained at ≤1.0 mg/dL for longer than 5 weeks. In some embodiments, the CRP value is maintained at ≤1.0 mg/dL for longer than 6 weeks. In some embodiments, the CRP value is maintained at ≤1.0 mg/dL for longer than 8 weeks. In some embodiments, the CRP value is maintained at ≤1.0 mg/dL for longer than 10 weeks. In some embodiments, the CRP value is maintained at ≤1.0 mg/dL for longer than 12 weeks. In some embodiments, the CRP value is maintained at ≤1.0 mg/dL for longer than 1 month. In some embodiments, the CRP value is maintained at ≤1.0 mg/dL for longer than 2 months. In some embodiments, the CRP value is maintained at ≤1.0 mg/dL for longer than 3 months. In some embodiments, the CRP value is maintained at ≤1.0 mg/dL for longer than 4 months. In some embodiments, the CRP value is maintained at ≤1.0 mg/dL for longer than 5 months. In some embodiments, the CRP value is maintained at ≤1.0 mg/dL for longer than 6 months. In some embodiments, the CRP value is maintained at ≤1.0 mg/dL for longer than 7 months. In some embodiments, the CRP value is maintained at ≤1.0 mg/dL for longer than 8 months. In some embodiments, the CRP value is maintained at ≤1.0 mg/dL for longer than 9 months. In some embodiments, the CRP value is maintained at ≤1.0 mg/dL for longer than 10 months. In some embodiments, the CRP value is maintained at ≤1.0 mg/dL for longer than 11 months. In some embodiments, the CRP value is maintained at ≤1.0 mg/dL for longer than 12 months. In some embodiments, the CRP value is maintained at ≤1.0 mg/dL for longer than 1 year. In some embodiments, the CRP level is maintained at ≤1.0 mg/dL for the above-indicated periods while the patient receives anti-IL1R1 antibody in absence of any concurrent therapy. In the foregoing embodiments, the CRP value is determined while the patient continues to receive a therapeutic dose of anti-IL1R1 antibody at an administration interval and treatment period according to the invention.

In some embodiments, the CRP value is maintained at ≤0.5 mg/dL for longer than 2 weeks. In some embodiments, the CRP value is maintained at ≤0.5 mg/dL for longer than 3 weeks. In some embodiments, the CRP value is maintained at ≤0.5 mg/dL for longer than 4 weeks. In some embodiments, the CRP value is maintained at ≤0.5 mg/dL for longer than 5 weeks. In some embodiments, the CRP value is maintained at ≤0.5 mg/dL for longer than 6 weeks. In some embodiments, the CRP value is maintained at ≤0.5 mg/dL for longer than 8 weeks. In some embodiments, the CRP value is maintained at ≤0.5 mg/dL for longer than 10 weeks. In some embodiments, the CRP value is maintained at ≤0.5 mg/dL for longer than 12 weeks. In some embodiments, the CRP value is maintained at ≤0.5 mg/dL for longer than 1 month. In some embodiments, the CRP value is maintained at ≤0.5 mg/dL for longer than 2 months. In some embodiments, the CRP value is maintained at ≤0.5 mg/dL for longer than 3 months. In some embodiments, the CRP value is maintained at ≤0.5 mg/dL for longer than 4 months. In some embodiments, the CRP value is maintained at ≤0.5 mg/dL for longer than 5 months. In some embodiments, the CRP value is maintained at ≤0.5 mg/dL for longer than 6 months. In some embodiments, the CRP value is maintained at ≤0.5 mg/dL for longer than 7 months. In some embodiments, the CRP value is maintained at ≤0.5 mg/dL for longer than 8 months. In some embodiments, the CRP value is maintained at ≤0.5 mg/dL for longer than 9 months. In some embodiments, the CRP value is maintained at ≤0.5 mg/dL for longer than 10 months. In some embodiments, the CRP value is maintained at ≤0.5 mg/dL for longer than 11 months. In some embodiments, the CRP value is maintained at ≤0.5 mg/dL for longer than 12 months. In some embodiments, the CRP value is maintained at ≤0.5 mg/dL for longer than 1 year. In some embodiments, the CRP level is maintained at ≤0.5 mg/dL for the above-indicated periods while the patient receives anti-IL1R1 antibody in absence of any concurrent therapy. In the foregoing embodiments, the CRP value is determined while the patient continues to receive a therapeutic dose of anti-IL1R1 antibody at an administration interval and treatment period according to the invention.

### Recurrence Rate or Recurrence-Free Period

In some embodiments, the effectiveness of treatment with an anti-IL1R1 antibody is determined by reduction of the risk of recurrence.

A recurrence-free period of survival typically means that the subject does not experience an episode or occurrence of one or more symptoms of pericarditis or a flare of inflammation during the period. A pericarditis recurrence or flare is usually indicated by any one or more of the following: an increase in the CRP level of ≥1 mg/dL in peripheral blood; or an increase in pain, determined in an NRS scale of ≥4; or occurrence of pericardial effusion (e.g., as determined by ECG or ECHO); a pericardial rub; or fever or any other symptomatic indication of the pericardial disease (e.g., as determined by PGIPS, PGA-PA, SF-36, EQ-5D-5L, or ISI).

In some embodiments, the administration of the anti-IL1R1 antibody results in a recurrence rate of 1% or less. In some embodiments, the administration of the anti-IL1R1 antibody results in a recurrence rate of 2% or less. In some embodiments, the administration of the anti-IL1R1 antibody results in a recurrence rate of 3% or less. In some embodiments, the administration of the anti-IL1R1 antibody results in a recurrence rate of 4% or less. In some embodiments, the administration of the anti-IL1R1 antibody results in a recurrence rate of 5% or less. In some embodiments, the administration of the anti-IL1R1 antibody results in a recurrence rate of 8% or less. In some embodiments, the administration of the anti-IL1R1 antibody results in a recurrence rate of 10% or less. In some embodiments, the administration of the anti-IL1R1 antibody results in a recurrence rate of 12% or less. In some embodiments, the administration of the anti-IL1R1 antibody results in a recurrence rate of 15% or less. In some embodiments, the administration of the anti-IL1R1 antibody results in a recurrence rate of 18% or less. In some embodiments, the administration of the anti-IL1R1 antibody results in a recurrence rate of 20% or less. In some embodiments, the administration of the anti-IL1R1 antibody results in a recurrence rate of 25% or less. In some embodiments, the administration of the anti-IL1R1 antibody results in a recurrence rate of 30% or less. In some embodiments, the administration of the anti-IL1R1 antibody results in a recurrence rate of 35% or less. In some embodiments, the administration of the anti-IL1R1 antibody results in a recurrence rate of 40% or less. In some embodiments, the administration of the anti-IL1R1 antibody results in a recurrence rate of 45% or less. In some embodiments, the administration of the anti-IL1R1 antibody results in a recurrence rate of 50% or less. In some embodiments, the administration of the anti-IL1R1 antibody results in a recurrence rate of substantially 0%.

In some embodiments, administration of the anti-IL1R1 antibody results in a median time-to-recurrence of greater than 80 weeks. In some embodiments, administration of the anti-IL1R1 antibody results in a median time-to-recurrence of greater than 70 weeks. In some embodiments, administration of the anti-IL1R1 antibody results in a median time-to-recurrence of greater than 60 weeks. In some embodiments, administration of the anti-IL1R1 antibody results in a median time-to-recurrence of greater than 55 weeks. In some embodiments, administration of the anti-IL1R1 antibody results in a median time-to-recurrence of greater than 50 weeks. In some embodiments, administration of the anti-IL1R1 antibody results in a median time-to-recurrence of greater than 45 weeks. In some embodiments, administration of the anti-IL1R1 antibody results in a median time-to-recurrence of greater than 40 weeks. In some embodiments, administration of the anti-IL1R1 antibody results in a median time-to-recurrence of greater than 35 weeks. In some embodiments, administration of the anti-IL1R1 antibody results in a median time-to-recurrence of greater than 30 weeks. In some embodiments, administration of the anti-IL1R1 antibody results in a median time-to-recurrence of greater than 25 weeks. In some embodiments, administration of the anti-IL1R1 antibody results in a median time-to-recurrence of greater than 20 weeks. In some embodiments, administration of the anti-IL1R1 antibody results in a median time-to-recurrence of greater than 15 weeks. In some embodiments, administration of the anti-IL1R1 antibody results in a median time-to-recurrence of greater than 10 weeks. In some embodiments, administration of the anti-IL1R1 antibody results in a median time-to-recurrence of greater than 8 weeks. In some embodiments, administration of the anti-IL1R1 antibody results in a median time-to-recurrence of greater than 4 weeks. In some embodiments, administration of the anti-IL1R1 antibody results in a median time-to-recurrence of greater than 1 week. In the foregoing embodiments, the medium time-to-recurrence is determined while the patient continues to receive a therapeutic dose of anti-IL1R1 antibody at an administration interval and treatment period according to the invention.

In some embodiments, administration of the anti-IL1R1 antibody results in a median time-to-recurrence of greater than 24 months. In some embodiments, administration of the anti-IL1R1 antibody results in a median time-to-recurrence of greater than 20 months. In some embodiments, administration of the anti-IL1R1 antibody results in a median time-to-recurrence of greater than 18 months. In some embodiments, administration of the anti-IL1R1 antibody results in a median time-to-recurrence of greater than 16 months. In some embodiments, administration of the anti-IL1R1 antibody results in a median time-to-recurrence of greater than 14 months. In some embodiments, administration of the anti-IL1R1 antibody results in a median time-to-recurrence of greater than 12 months. In some embodiments, administration of the anti-IL1R1 antibody results in a median time-to-recurrence of greater than 10 months. In some embodiments, administration of the anti-IL1R1 antibody results in a median time-to-recurrence of greater than 8 months. In some embodiments, administration of the anti-IL1R1 antibody results in a median time-to-recurrence of greater than 6 months. In some embodiments, administration of the anti-IL1R1 antibody results in a median time-to-recurrence of greater than 5 months. In some embodiments, administration of the anti-IL1R1 antibody results in a median time-to-recurrence of greater than 4 months. In some embodiments, administration of the anti-IL1R1 antibody results in a median time-to-recurrence of greater than 3 months. In some embodiments, administration of the anti-IL1R1 antibody results in a median time-to-recurrence of greater than 2 months. In some embodiments, administration of the anti-IL1R1 antibody results in a median time-to-recurrence of greater than 1 month.

In some embodiments, administration of the anti-IL1R1 antibody results in a median time-to-recurrence of greater than 5 years. In some embodiments, administration of the anti-IL1R1 antibody results in a median time-to-recurrence of greater than 4 years. In some embodiments, administration of the anti-IL1R1 antibody results in a median time-to-recurrence of greater than 3 years. In some embodiments, administration of the anti-IL1R1 antibody results in a median time-to-recurrence of greater than 2 years. In some embodiments, administration of the anti-IL1R1 antibody results in a median time-to-recurrence of greater than 1 year. In the foregoing embodiments, the medium time-to-recurrence is determined while the patient continues to receive a therapeutic dose of anti-IL1R1 antibody at an administration interval and treatment period according to the invention.

In some embodiments, the median time-to-recurrence could not be determined due to a limited number of recurrence events. In some embodiments, the limited number of recurrence events is less than 3. In some embodiments, the limited number of recurrence events is less than 2. In some embodiments, the limited number of recurrence events is less than 1. In some embodiments, the limited number of recurrence events is substantially 0.

In some embodiments, the effectiveness of treatment with an anti-IL1R1 antibody is determined by a hazard ratio for recurrence when comparing a treatment group to a placebo group. A low hazard ratio indicates a significantly reduced risk of pericarditis recurrence with the treatment group; meaning the treatment (with an anti-IL1R1) significantly lowers the chance of a pericarditis flare-up compared to placebo or control.

In some embodiments, the administration of the anti-IL1R1 antibody as compared to the control results in a hazard ratio of less than 0.5. In some embodiments, the administration of the anti-IL1R1 antibody as compared to the control results in a hazard ratio of less than 0.4. In some embodiments, the administration of the anti-IL1R1 antibody as compared to the control results in a hazard ratio of less than 0.3. In some embodiments, the administration of the anti-IL1R1 antibody as compared to the control results in a hazard ratio of less than 0.2. In some embodiments, the administration of the anti-IL1R1 antibody as compared to the control results in a hazard ratio of less than 0.1. In some embodiments, the administration of the anti-IL1R1 antibody as compared to the control results in a hazard ratio of less than 0.05. In some embodiments, the administration of the anti-IL1R1 antibody as compared to the control results in a hazard ratio of less than 0.04. In some embodiments, the administration of the anti-IL1R1 antibody as compared to the control results in a hazard ratio of less than 0.03. In some embodiments, the administration of the anti-IL1R1 antibody as compared to the control results in a hazard ratio of less than 0.02. In some embodiments, the administration of the anti-IL1R1 antibody as compared to the control results in a hazard ratio of less than 0.01. In some embodiments, the administration of the anti-IL1R1 antibody as compared to the control results in a hazard ratio of less than 0.005.

In some embodiments, the effectiveness of treatment with an anti-IL1R1 antibody is determined by a recurrence-free period during or subsequent to the treatment. In some embodiments, a subject continues to receive a therapeutic dose of anti-IL1R1 antibody at an administrative interval and treatment period in accordance with the invention during the recurrence-free period. In some embodiments, a subject may be gradually weaned off an anti-IL1R1 antibody treatment during the recurrence-free period.

In some embodiments, the effectiveness of treatment with an anti-IL1R1 antibody is determined by the time period of recurrence-free survival (for examples, Days to flare). In some embodiments, the recurrence-free survival period is at least 30 days, 40 days, 50 days, 60 days, 70 days, 80 days, or 90 days from the first administration of the anti-IL1R1 antibody. In some embodiments, the recurrence-free survival period is at least 91 days, 92 days, 93 days, 94 days, 95 days, 96 days, 97 days, 98 days, 99 days, 100 days from the first administration of the anti-IL1R1 antibody. In some embodiments, the recurrence-free survival period is at least 105 days, or at least 110 days, or at least 115 days, or at least 120 days, or at least 130 days, or at least 140 days, or at least 150 days. In some embodiments, the recurrence-free survival period is at least 200 days or more, while receiving anti-IL1R1 antibody at an administration interval (e.g., a weekly dose) and treatment period according to the invention.

In some embodiments, the recurrence-free survival period is at least 30 days, 40 days, 50 days, 60 days, 70 days, or at least 80 days from the withdrawal of a pain medicine, or NSAID, while receiving a therapeutically effective dose of the anti-IL1R1 antibody according to the invention. In some embodiments, the recurrence-free survival period from the withdrawal of NSAID is at least 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 110 days, 120 days, 130 days, 140 days, 150 days, 160 days, 170 days, 180 days, 190 days or 200 days or more, while receiving anti-IL1R1 antibody at an administration interval (e.g., a weekly dose) and treatment period according to the invention.

In some embodiments, the recurrence-free survival period is at least 50 days from the withdrawal of a corticosteroid, while receiving a therapeutically effective dose of the anti-IL1R1 antibody according to the invention. In some embodiments the flare-free survival period from the withdrawal of NSAID is at least 55 days, 60 days, 65 days, 70 days, 75 days, 80 days, 85 days, 90 days, 95 days, 100 days, 110 days, 120 days, 130 days, 140 days, 150 days, 160 days, 170 days, 180 days, 190 days or 200 days or more, while receiving anti-IL1R1 antibody at an administration interval (e.g., a weekly dose) and treatment period according to the invention.

### Median Time to Monotherapy

In some embodiments, administration of an anti-IL1R1 antibody in successful taper of steroids and other concurrent therapies, while the subject continues to receive an anti-IL1R1 antibody protein at an administration interval and treatment period according to the invention. In some embodiments the median time to anti-IL1R1 antibody monotherapy refers to successful taper of background medications, concurrent therapies or standard of care.

In some embodiments, the median time to anti-IL1R1 antibody monotherapy is 1 week or less. In some embodiments, the median time to anti-IL1R1 antibody monotherapy is 2 weeks or less. In some embodiments, the median time to anti-IL1R1 antibody monotherapy is 3 weeks or less. In some embodiments, the median time to anti-IL1R1 antibody monotherapy is 4 weeks or less. In some embodiments, the median time to anti-IL1R1 antibody monotherapy is 6 weeks or less. In some embodiments, the median time to anti-IL1R1 antibody monotherapy is 8 weeks or less. In some embodiments, the median time to anti-IL1R1 antibody monotherapy is 10 weeks or less. In some embodiments, the median time to anti-IL1R1 antibody monotherapy is 12 weeks or less. In some embodiments, the median time to anti-IL1R1 antibody monotherapy is 14 weeks or less. In some embodiments, the median time to anti-IL1R1 antibody monotherapy is 16 weeks or less. In some embodiments, the median time to anti-IL1R1 antibody monotherapy is 18 weeks or less. In some embodiments, the median time to anti-IL1R1 antibody monotherapy is 20 weeks or less. In some embodiments, the median time to anti-IL1R1 antibody monotherapy is 22 weeks or less. In some embodiments, the median time to anti-IL1R1 antibody monotherapy is 24 weeks or less.

In some embodiments, administration of an anti-IL1R1 antibody results in tapering off of background medications, concurrent therapies, or standard of care treatment (e.g., NSAID, colchicine, corticosteroids in any combination) while receiving an anti-IL1R1 antibody monotherapy.

In some embodiments, administration of an anti-IL1R1 antibody results in cessation of background medications, concurrent therapies, or standard of care treatment at 1 week from the first administration of the anti-IL1R1 antibody. In some embodiments, administration of an anti-IL1R1 antibody results in cessation of background medications, concurrent therapies, or standard of care treatment at 2 weeks from the first administration of the anti-IL1R1 antibody. In some embodiments, administration of an anti-IL1R1 antibody results in cessation of background medications, concurrent therapies, or standard of care treatment at 3 weeks from the first administration of the anti-IL1R1 antibody. In some embodiments, administration of an anti-IL1R1 antibody results in cessation of background medications, concurrent therapies, or standard of care treatment at 4 weeks from the first administration of the anti-IL1R1 antibody. In some embodiments, administration of an anti-IL1R1 antibody results in cessation of background medications, concurrent therapies, or standard of care treatment at 5 weeks from the first administration of the anti-IL1R1 antibody. In some embodiments, administration of an anti-IL1R1 antibody results in cessation of background medications, concurrent therapies, or standard of care treatment at 6 weeks from the first administration of the anti-IL1R1 antibody. In some embodiments, administration of an anti-IL1R1 antibody results in cessation of background medications, concurrent therapies, or standard of care treatment at 7 weeks from the first administration of the anti-IL1R1 antibody. In some embodiments, administration of an anti-IL1R1 antibody results in cessation of background medications, concurrent therapies, or standard of care treatment at 8 weeks from the first administration of the anti-IL1R1 antibody. In some embodiments, administration of an anti-IL1R1 antibody results in cessation of background medications, concurrent therapies, or standard of care treatment at 10 weeks from the first administration of the anti-IL1R1 antibody. In some embodiments, administration of an anti-IL1R1 antibody results in cessation of background medications, concurrent therapies, or standard of care treatment at 12 weeks from the first administration of the anti-IL1R1 antibody. In some embodiments, administration of an anti-IL1R1 antibody results in cessation of background medications, concurrent therapies, or standard of care treatment at 14 weeks from the first administration of the anti-IL1R1 antibody. In some embodiments, administration of an anti-IL1R1 antibody results in cessation of background medications, concurrent therapies, or standard of care treatment at 16 weeks from the first administration of the anti-IL1R1 antibody. In some embodiments, administration of an anti-IL1R1 antibody results in cessation of background medications, concurrent therapies, or standard of care treatment at 18 weeks from the first administration of the anti-IL1R1 antibody. In some embodiments, administration of an anti-IL1R1 antibody results in cessation of background medications, concurrent therapies, or standard of care treatment at 20 weeks from the first administration of the anti-IL1R1 antibody. In some embodiments, administration of an anti-IL1R1 antibody results in cessation of background medications, concurrent therapies, or standard of care treatment at 22 weeks from the first administration of the anti-IL1R1 antibody. In some embodiments, administration of an anti-IL1R1 antibody results in cessation of background medications, concurrent therapies, or standard of care treatment at 24 weeks from the first administration of the anti-IL1R1 antibody. In the foregoing embodiments, the time to cessation of background medications, concurrent therapies, or standard of care treatment is determined while the patient continues to receive a therapeutic dose of anti-IL1R1 antibody at an administration interval and treatment period according to the invention.

In some embodiments, administration of an anti-IL1R1 antibody results in a successful cessation of background medications, concurrent therapies, or standard of care treatment for longer than 2 weeks. In some embodiments, administration of an anti-IL1R1 antibody results in a successful cessation of background medications, concurrent therapies, or standard of care treatment for longer than 4 weeks. In some embodiments, administration of an anti-IL1R1 antibody results in a successful cessation of background medications, concurrent therapies, or standard of care treatment for longer than 6 weeks. In some embodiments, administration of an anti-IL1R1 antibody results in a successful cessation of background medications, concurrent therapies, or standard of care treatment for longer than 8 weeks. In some embodiments, administration of an anti-IL1R1 antibody results in a successful cessation of background medications, concurrent therapies, or standard of care treatment for longer than 10 weeks. In some embodiments, administration of an anti-IL1R1 antibody results in a successful cessation of background medications, concurrent therapies, or standard of care treatment for longer than 12 weeks. In some embodiments, administration of an anti-IL1R1 antibody results in a successful cessation of background medications, concurrent therapies, or standard of care treatment for longer than 1 month. In some embodiments, administration of an anti-IL1R1 antibody results in a successful cessation of background medications, concurrent therapies, or standard of care treatment for longer than 2 months. In some embodiments, administration of an anti-IL1R1 antibody results in a successful cessation of background medications, concurrent therapies, or standard of care treatment for longer than 4 months. In some embodiments, administration of an anti-IL1R1 antibody results in a successful cessation of background medications, concurrent therapies, or standard of care treatment for longer than 6 months. In some embodiments, administration of an anti-IL1R1 antibody results in a successful cessation of background medications, concurrent therapies, or standard of care treatment for longer than 8 months. In some embodiments, administration of an anti-IL1R1 antibody results in a successful cessation of background medications, concurrent therapies, or standard of care treatment for longer than 10 months. In some embodiments, administration of an anti-IL1R1 antibody results in a successful cessation of background medications, concurrent therapies, or standard of care treatment for longer than 12 months. In some embodiments, administration of an anti-IL1R1 antibody results in a successful cessation of background medications, concurrent therapies, or standard of care treatment for longer than 1 year. In the foregoing embodiments, the duration of successful cessation of background medications, concurrent therapies, or standard of care treatment is determined while the patient continues to receive a therapeutic dose of anti-IL1R1 antibody at an administration interval and treatment period according to the invention.

In some embodiments, the patient continues to receive an anti-IL1R1 antibody at a therapeutic dose and administration interval in accordance to the invention. In some embodiments, the subject continues to receive an anti-IL1R1 antibody without any concurrent therapy.

### Short Form Health Survey (SF-36)

In some embodiments, the effectiveness of treatment with an anti-IL1R1 antibody is determined by SF-36.

The Short Form Health Survey (SF-36) is a widely used tool to measure a person's overall health status and quality of life. It consists of 36 questions that assess eight different dimensions of health, which can help provide a comprehensive view of an individual's physical and mental well-being. The SF-36 scores are weighted sums of the questions in each dimension, and they from 0-100. The eight dimensions are:
Vitality
Physical functioning
Bodily pain
General health perceptions
Physical role functioning
Emotional role functioning
Social role functioning
Mental Health

In some embodiments, the administration of the anti-IL1R1 antibody results in an improvement as compared to a control, as assessed by SF-36. In some embodiments, an improvement in the SF-36 is indicated by a higher score.

In some embodiments, the administration of the anti-IL1R1 antibody results in an improvement in vitality as compared to a control. In some embodiments, the administration of the anti-IL1R1 antibody results in an improvement in physical functioning as compared to a control. In some embodiments, the administration of the anti-IL1R1 antibody results in an improvement in bodily pain as compared to a control. In some embodiments, the administration of the anti-IL1R1 antibody results in an improvement in general health perceptions as compared to a control. In some embodiments, the administration of the anti-IL1R1 antibody results in an improvement in physical role functioning as compared to a control. In some embodiments, the administration of the anti-IL1R1 antibody results in an improvement in emotional role functioning as compared to a control. In some embodiments, the administration of the anti-IL1R1 antibody results in an improvement in social role functioning as compared to a control. In some embodiments, the administration of the anti-IL1R1 antibody results in an improvement in mental health as compared to a control.

### Adverse Events

Adverse effects related to the treatment of pericarditis can include injection-site reaction, upper respiratory tract infection, headache, nausea, vomiting, diarrhea, sinusitis, arthralgia, flu-like symptoms, abdominal pain, pyrexia, herpes, transaminase elevation, ischemic optic neuropathy and nasopharyngitis.

In some embodiments, administration of an anti-IL1R1 antibody does not result in an adverse event in the patient. In some embodiments, the administration of the anti-IL1R1 antibody does not result in a dose-limiting adverse events (AE). In some embodiments, the administration of the anti-IL1R1 antibody does not result in a dose-related adverse events (AE). In some embodiments, the administration of the anti-IL1R1 antibody does not result in a dose-limiting or dose-related adverse events (AE). In some embodiments, administration of an anti-IL1R1 antibody does not result in serious adverse events in the patient. In some embodiments, administration of an anti-IL1R1 antibody does not result in one or more of injection-site reaction, worsening of rheumatoid arthritis, upper respiratory tract infection, headache, nausea, vomiting, diarrhea, sinusitis, ischemic optic neuropathy, arthralgia, flu-like symptoms, abdominal pain, pyrexia, herpes, transaminase elevation and nasopharyngitis.

In some embodiments, administration of an anti-IL1R1 antibody does not result in an injection-site reaction. In some embodiments, administration of an anti-IL1R1 antibody does not result in upper respiratory tract infection. In some embodiments, administration of an anti-IL1R1 antibody does not result in headache. In some embodiments, administration of an anti-IL1R1 antibody does not result in nausea. In some embodiments, administration of an anti-IL1R1 antibody does not result in vomiting. In some embodiments, administration of an anti-IL1R1 antibody does not result in diarrhea. In some embodiments, administration of an anti-IL1R1 antibody does not result in sinusitis. In some embodiments, administration of an anti-IL1R1 antibody does not result in arthralgia. In some embodiments, administration of an anti-IL1R1 antibody does not result in flu-like symptoms. In some embodiments, administration of an anti-IL1R1 antibody does not result in abdominal pain. In some embodiments, administration of an anti-IL1R1 antibody does not result in pyrexia. In some embodiments, administration of an anti-IL1R1 antibody does not result in herpes. In some embodiments, administration of an anti-IL1R1 antibody does not result in transaminase elevation. In some embodiments, administration of an anti-IL1R1 antibody does not result in X. In some embodiments, administration of an anti-IL1R1 antibody does not result in nasopharyngitis. In some embodiments, administration of an anti-IL1R1 antibody does not result in ischemic optic neuropathy.

In some embodiments, the various safety assessments include pharmacokinetic and pharmacodynamic monitoring, including but not limited to: physical examination, measurement of vital signs, monitoring adverse event (AE), monitoring chest X-ray and screening for signs of tuberculosis.

### Other Measurement

In some embodiments, administration of an anti-IL1R1 antibody results in improved cardiac function, such as decreased or ameliorated pericardiac effusion, compared to a control. Pericardial effusion (also known as “fluid around the heart”) is an abnormal accumulation of fluid in the pericardial cavity that is indicative of inflammation, which leads to an increased intrapericardial pressure and can negatively affect heart function. Cardiac health parameters, particularly pericardiac effusion, may be evaluated by echocardiography (ECHO), cardiac magnetic resonance imaging (MRI), and/or CT scanning. In some embodiments, administration of an anti-IL1R1 antibody results in a decrease, absence or amelioration of pericardiac effusion.

A control is a reference cardiac parameter such as a reference pericardiac effusion level measured in a patient with comparable disease status but without treatment. For example, a control may be a baseline cardiac function such as a baseline pericardiac effusion measured in the patient being treated prior to the treatment. A control may also be a reference cardiac parameter such as a reference pericardiac effusion level measured in a patient with comparable disease status but treated with a placebo. In some embodiments, a control may be a reference value or graph indicative of cardiac parameters such as pericardiac effusion in a patient with comparable disease status without treatment based on historical data. In some embodiments, a control may be a reference value or graph indicative of a cardiac parameter such as pericardiac effusion in a subject with comparable disease status who receives a standard of care therapy, in absence of an anti-IL1R1 antibody administration.

In some embodiments, administration of an anti-IL1R1 antibody results in improvement of cardiac parameters as indicated by ECG measurements. A reduction in the ST elevation and/or reduction in the depression after administration of the in an anti-IL1R1 antibody is considered an improvement in the cardiac parameter.

In some embodiments, administration of an anti-IL1R1 antibody results in improvement of cardiac effusion as determined by echocardiography (ECHO).

In some embodiments, administration of an anti-IL1R1 antibody results in improvement of cardiac parameters as determined by CT scanning.

In some embodiments, administration of an anti-IL1R1 antibody results in improvement of cardiac parameters as determined by cardiac magnetic resonance imaging (MRI). In some embodiments the cardiac parameters as determined by cardiac magnetic resonance imaging include pericardial delayed hyperenhancement, pericardial effusion or pericardial effusion size, and combinations thereof.

In some embodiments, administration of an anti-IL1R1 antibody results in improvement of QoL scores. Typically, QoL scores comprise one or more assessments selected from: Patient Global Impression of Pericarditis Severity (PGIPS); Physician Global Assessment of Pericarditis Activity (PGA-PA); 36-Item Short Form Health Survey (SF-36); 5-Level EuroQoL-5D (EQ-5D-5L) and Insomnia severity Index (ISI).

In some embodiments, the effectiveness of treatment using an anti-IL1R1 antibody is determined by Patient Global Impression of Pericarditis Severity (PGIPS), and/or Physician Global Assessment of Pericarditis Activity (PGA-PA). The PGIPS is a single-item patient reported outcome (PRO) measure that assesses the subject's impression of overall severity of pericarditis symptoms at the time the questionnaire is administered, using a 7-point rating scale ranging from absent (no recurrent pericarditis symptoms) to very severe (recurrent pericarditis symptoms cannot be ignored). The PGA-PA is a single-item clinician-reported outcome measure that investigators use to rate their impression of the patient's overall pericarditis disease activity at the time the assessment is completed, using a 7-point rating scale ranging from absent to very severe. In some embodiments, administration of an anti-IL1R1 antibody results in an improved PGIPS and/or PGA-PA compared to a control.

In some embodiments, at least 50% of the subjects undergoing treatment with an anti-IL1R1 antibody report an absence or minimal pericarditis symptoms as determined by PGIPS. In some embodiments, at least 75% of the subjects undergoing treatment with an anti-IL1R1 antibody report an absence or minimal pericarditis symptoms as determined by PGIPS. In some embodiments, at least 80% of the subjects undergoing treatment with an anti-IL1R1 antibody report an absence or minimal pericarditis symptoms as determined by PGIPS. In some embodiments, at least 90% of the subjects undergoing treatment with an anti-IL1R1 antibody report an absence or minimal pericarditis symptoms as determined by PGIPS. In some embodiments, at least 95% of the subjects undergoing treatment with an anti-IL1R1 antibody report an absence or minimal pericarditis symptoms as determined by PGIPS. In some embodiments, 97%, 98%, 99% or 100% of the subjects undergoing treatment with an anti-IL1R1 antibody report an absence or minimal pericarditis symptoms as determined by PGIPS.

In some embodiments, at least 50% of the subjects receiving an anti-IL1R1 antibody show an absence or minimal pericarditis symptoms as determined by PGA-PA. In some embodiments, at least 75% of the subjects undergoing treatment with an anti-IL1R1 antibody show an absence or minimal pericarditis symptoms as determined by PGA-PA. In some embodiments, at least 80% of the subjects undergoing treatment with an anti-IL1R1 antibody show an absence or minimal pericarditis symptoms as determined by PGA-PA. In some embodiments, at least 90% of the subjects undergoing treatment with an anti-IL1R1 antibody show an absence or minimal pericarditis symptoms as determined by PGA-PA. In some embodiments, at least 95% of the subjects undergoing treatment with an anti-IL1R1 antibody show an absence or minimal pericarditis symptoms as determined by PGA-PA. In some embodiments, 96%, 97%, 98%, 99% or 100% of the subjects undergoing treatment with an anti-IL1R1 antibody show an absence or minimal pericarditis symptoms as determined by PGA-PA.

In some embodiments, the effectiveness of the treatment using an anti-IL1R1 antibody is determined by a 5-Level EuroQoL-5D (EQ-5D-5L) (additional information in: www.euroqol.org). The EQ-5D-5L is a standardized instrument developed by the EuroQol Group as a measure of health-related quality of life that can be used in assessing a wide range of health conditions and treatments. The EQ-5D-5L includes a descriptive system and the EQ VAS. The descriptive system comprises 5 dimensions: mobility, self-care, usual activities, pain/discomfort and anxiety/depression. The rating scale records the subject's self-rated health on a vertical Visual Analog Scale (VAS). The scores on these 5 dimensions can be presented as a health profile or can be converted to a single summary index number (utility) reflecting preferability compared to other health profiles (euroqol.org/eq-5d-instruments).

In some embodiments, the EQ-5D-5L is collected in subjects ≥18 years or older.

In some embodiments, administration of an anti-IL1R1 antibody results in an improved EQ-5D-5L. In some embodiments, at least 50% of the subjects receiving an anti-IL1R1 antibody show a clinically relevant improved EQ-5D-5L score. In some embodiments, at least 75% of the subjects undergoing treatment with an anti-IL1R1 antibody show a clinically relevant improved EQ-5D-5L score. In some embodiments, at least 80% of the subjects undergoing treatment with an anti-IL1R1 antibody show a clinically relevant improved EQ-5D-5L score. In some embodiments, at least 90% of the subjects undergoing treatment with an anti-IL1R1 antibody show a clinically relevant improved EQ-5D-5L score. In some embodiments, at least 95% of the subjects undergoing treatment with an anti-IL1R1 antibody show a clinically relevant improved EQ-5D-5L score. In some embodiments, 96%, 97%, 98%, 99% or 100% of the subjects undergoing treatment with an anti-IL1R1 antibody a clinically relevant improved EQ-5D-5L score.

In some embodiments, a control for a QoL measurements indicated in the preceding sections, is a baseline QoL scores determined in the subject prior to the treatment. In some embodiments, a control is reference QoL scores in a subject with comparable disease status but treated with a placebo. In some embodiments, a control is a reference indicative of QoL scores in a subject with comparable disease status without treatment. In some embodiments, a control is indicative of the QoL when a subject having the disease receives a standard of care therapy, in absence of IL an anti-IL1R1 antibody administration.

In some embodiments, administration of an anti-IL1R1 antibody results in an improved Insomnia Severity Index (ISI). The ISI is a 7-item self-report questionnaire assessing the nature, severity, and impact of insomnia. The usual recall period is the “last 2 weeks” and the dimensions evaluated are severity of sleep onset, sleep maintenance, early morning awakening problems, sleep dissatisfaction, interference of sleep difficulties with daytime functioning, noticeability of sleep problems by others, and distress caused by the sleep difficulties. A 5-point Likert scale is used to rate each item (e.g., 0=no problem; 4=very severe problem), yielding a total score ranging from 0 to 28. The total score is interpreted as follows: no clinically significant insomnia (0-7); subthreshold insomnia (8-14); clinical (moderate) insomnia (15-21); and clinical (severe) insomnia (22-28) (Morin et al., Sleep. 2011; 34(5):601-608). In some embodiments, the ISI is collected in subjects ≥18 years or older. In some embodiments, the ISI in a subject after administration of an anti-IL1R1 antibody is improved to less than 14, or between 8-14, or less than 7. In some embodiments, administration of an anti-IL1R1 antibody results in the ISI of less than or equal to 7 in the subject being treated. In some embodiments, ISI score is less than or equal to 7 in at least 50% of the subjects who receive an anti-IL1R1 antibody. In some embodiments, ISI score is less than or equal to 7 in at least 75% of the subjects who receive an anti-IL1R1 antibody. In some embodiments, ISI score is less than or equal to 7 in at least 80%, 90%, 95%, 96%, 97%, 98%, 99% or 100% of the subjects who receive an anti-IL1R1 antibody.

In some embodiments, the effectiveness of treatment with an anti-IL1R1 antibody is determined by a reduced risk of pericarditis recurrence compared to a no-treatment or placebo control. In some embodiments, administration of an anti-IL1R1 antibody results in reduced risk of pericarditis recurrence of less than 50%, less than 40%, less than 30%, less than 20%, less than 10%, less than 5%, less than 4%, less than 3%, less than 2%, or less than 1% or less than 0.5% or less than 0.25% or less than 0.1% based on statistical analysis of patients population being treated with an anti-IL1R1 antibody according to the present invention.

### EXAMPLES

While certain methods of the present invention have been described with specificity in accordance with certain embodiments, the following examples serve only to illustrate the methods of the invention and are not intended to limit the same.

### Example 1. Single Ascending Dose Study of KPL-387 for Treatment of Recurrent Pericarditis

The study in this example was a Phase I single ascending dose (SAD) clinical trial designed to evaluate the safety, tolerability, pharmacokinetics, and pharmacodynamics of subcutaneously administered KPL-387, an anti-IL1R1 antibody, for the treatment of recurrent pericarditis. The CDR sequences of KPL-387 are shown in Table 7. The heavy and light chain amino acid sequences of KPL-387 are shown in Table 8.

TABLE 7
KPL-387 CDR sequences

| CDR | | | | | | |
|---|---|---|---|---|---|---|
| numbering | SEQ ID NO | | | | | |
| system | HCDR1 | HCDR2 | HCDR3 | LCDR1 | LCDR2 | LCDR3 |
| Kabat | 1 | 2 | 3 | 4 | 5 | 6 |
| Chothia | 12 | 13 | 3 | 4 | 5 | 6 |
| IMGT | 14 | 15 | 19 | 16 | 17 | 6 |

TABLE 8
KPL-387 heavy and light chain sequences

| Amino acid sequence | SEQ ID NO |
|---|---|
| V_{H} | 7 |
| V_{L} | 8 |
| Heavy chain | 10 |
| Light chain | 11 |

Single-dose subcutaneous administrations of KPL-387 were given at dosages of 1 mg, 3 mg, 10 mg, 30 mg, 100 mg, 300 mg and 600 mg in Cohorts 1, 2, 3, 4, 5, 6, and 7 respectively in healthy human participants. No dose-limiting adverse events (AE) and no dose-related AE were observed.

Pharmacokinetic (PK) and pharmacodynamic (PD) analyses were also performed. The pharmacodynamic profile was assessed by evaluating the duration of maintaining a KPL-387 serum concentration resulting in 90% (EC₉₀) inhibition of IL-1-stimulated IL-6 production in primary human cells (0.5 μg/mL). Of note, the target of KPL-387 is not expected to be highly expressed, nor is IL-6 production anticipated to be present, in healthy volunteers; as such this value is theoretical as envisioned in the future setting of a potential inflammatory disease state.

Table 9 shows the observed PK and predicted PD parameter. As can be seen from the data, the lower dose cohorts (1 mg, 3 mg, 10 mg, and 30 mg) showed low to undetectable concentrations of KPL-387 in serum and low to undetectable time maxima in serum. The observed PK profiles were compared to the predicted PK profiles derived from a non-human primate (non-clinical) population PK (NHP Pop PK) model, which was built by incorporating target-mediated drug disposition (TMDD) values from patients with active inflammatory diseases. IL-1R1 value (2.23 nM) was imputed into the NHP Pop PK model to reflect magnitude of TMDD in active inflammation (Ngo et al., 2020).

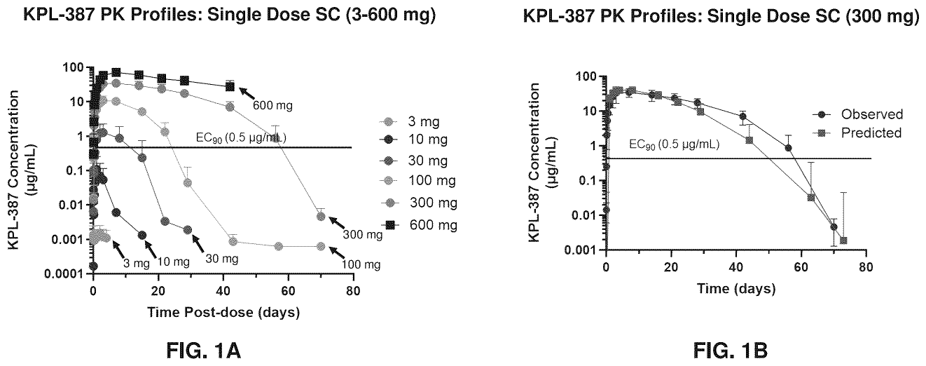

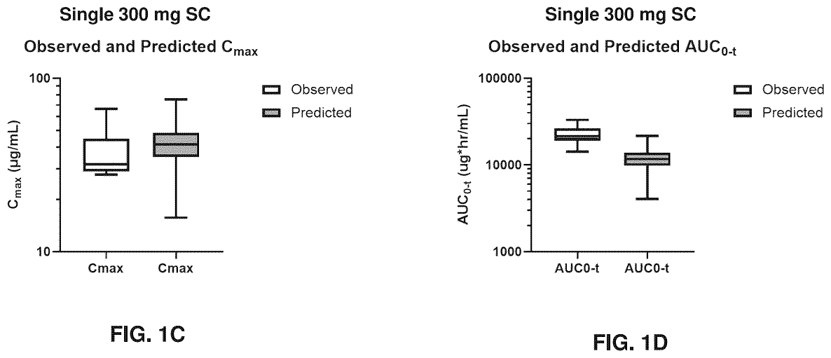

As illustrated in Table 9 and FIG. 1A, a single 30 mg SC dose of KPL-387 maintained mean serum concentrations of KPL-387 above the EC₉₀ for approximately 7 days. A single 100 mg SC dose of KPL-387 maintained mean serum concentrations of KPL-387 above the EC₉₀ for approximately 21 days. A single 300 mg SC dose of KPL-387 maintained mean serum concentrations of KPL-387 above the EC₉₀ for greater than 56 days. A single 300 mg SC dose of KPL-387 maintained serum concentrations of KPL-387 above the EC₉₀ for a minimum of 42 days in all 6 subjects tested, and the KPL-387 serum concentrations remained above the EC₉₀ at Day 56 in 3 of 6 subjects. In the other 3 subjects that received a 300 mg dose, serum concentrations of KPL-387 fell below EC₉₀ at approximately Day 45 to 56. A single 600 mg SC dose of KPL-387 maintained mean serum concentrations of KPL-387 above the EC₉₀ for greater a minimum of 42 days in all 6 subjects. As shown in FIG. 1B, in the 300 mg cohort, observed mean KPL-387 serum concentration-time values were higher than predicted values at later time points (e.g., Day 28 to Day 56). The slower “observed” KPL-387 clearance (longer persistence) at later timepoints in FIG. 1B is likely attributable to lower TMDD and lower IL-1R levels inherent in healthy volunteers, compared to the “predicted” NHP Pop PK-predicted model, which was built incorporating TMDD values from patients with active inflammatory diseases. FIG. 1C and FIG. 1D show box and whisker plots of observed PK parameters and NHP Pop PK-predicted values in the 300 mg cohort up to 70 days. As shown in FIG. 1C, the predicted mean KPL-387 Cₘₐₓ is in good agreement with observed value. FIG. 1D shows the higher observed KPL-387 AUC₀₋ₜ, compared to the predicted value. The mean and median observed AUC₀₋ₜ was approximately 2-fold higher compared to NHP Pop PK model-predicted values. The mean and median observed t_{1/2} was approximately 2-fold higher compared to NHP Pop PK model-predicted values.

TABLE 9
KPL-387-C101 First in Human Study Dose Levels and Observed PK and
Predicted PD Parameters

| | | | | | | Predicted |
|---|---|---|---|---|---|---|
| | | | | | | Stimulated |
| | | | | | | IL-6 |
| | Frequency | | | | | Production |
| | (single or | Route of | Dose | Median | Median | at Median |
| | multiple | Adminis- | level | Cₘₐₓ | Tₘₐₓ | Cₘₐₓ |
| Cohort | dose) | tration | (mg) | (μg/mL) | (h) | (%) |
| 1 | Single | SC | 1 mg | ND | ND | ND |
| 2 | Single | SC | 3 mg | ND | ND | ND |
| 3 | Single | SC | 10 mg | 0.05 | 48 | 57 |
| 4 | Single | SC | 30 mg | 0.94 | 48 | 14.1 |
| 5 | Single | SC | 100 mg | 10.0 | 168 | 10.1 |
| 6 | Single | SC | 300 mg | 32.0 | 168 | 9.9 |

Abbreviations: Cₘₐₓ = maximal concentration, IL-6 = interleukin 6, ND = not determined (insufficient values needed to derive parameter), SC = subcutaneous, Tₘₐₓ = time to maximal concentration.
Predicted Stimulated IL-6 production at median Cₘₐₓ = 100 − (90.42 × Cₘₐₓ/0.05476 + Cₘₐₓ)

Based on the existing data, the KPL-387 300 mg SC q2 wk, 300 mg SC q4 wk, and potentially the 100 mg SC q2 wk dose regimens are predicted to maintain EC₉₀ levels in most participants throughout the respective dosing cycles, in support of the objective of q4 wk administration, anticipated to be at the 300 mg SC dose level. The 100 mg SC q4 wk dose level is predicted to saturate the IL-1 receptor for the first 2 weeks of the 4-week dose interval, and assessment of pericardial pain, subclinical CRP elevations, or even pericarditis recurrences will be assessed and managed as part of the assessment of the minimum efficacious dose level for multiple-ascending dose (MAD) study.

### Example 2. Multiple Ascending Dose Study of KPL-387 for Treatment of Recurrent Pericarditis

The study in this example was a Phase I multiple ascending dose (MAD) clinical trial to evaluate the safety, tolerability, pharmacokinetics, and pharmacodynamics of multiple, subcutaneously administered doses of KPL-387, an anti-IL1R1 antibody, for the treatment of recurrent pericarditis.

Subcutaneous administrations of KPL-387 were given at dosages of 100 mg once every four weeks for 3 doses, 300 mg once every four weeks for 3 doses, and 300 mg once every two weeks for 6 doses in healthy human participants, and pharmacokinetic and pharmacodynamic analyses were performed.

The pharmacodynamic profile was assessed by evaluating the duration of maintaining a KPL-387 serum concentration resulting in 90% (EC₉₀) inhibition of IL-1-stimulated IL-6 production in primary human cells (0.5 μg/mL).

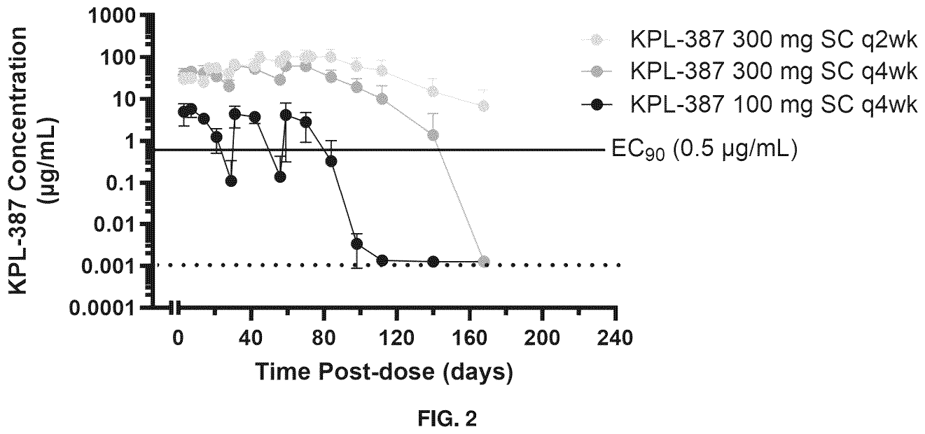

FIG. 2 shows the observed KPL-387 serum concentration over time for administrations of 100 mg once every four weeks (3 doses), 300 mg once every four weeks (3 doses), and 300 mg once every two weeks (6 doses). As shown, 300 mg SC doses of KPL-387 once every two weeks and once every four weeks observed serum concentrations of KPL-387 were maintained above the EC₉₀ for a minimum of 160 days and 140 days, respectively.

### Example 3. Multi-Ascending Dose (MAD) Study of KPL-387 for Treatment in Participants with Recurrent Pericarditis

This example outlines the exemplary Phase 2/3 clinical studies (Part A and Part B, respectively) to assess efficacy and safety of KPL-387 for treating patients with recurrent pericarditis (RP). The Long-Term Extension (LTE) study to assess the long-term efficacy of KPL-387 is also outlined as Part C.

### Objectives and Primary & Secondary Endpoints—Part A (Phase 2)

| Primary Objective - Part A | Primary Efficacy Endpoint - Part A |
|---|---|
| To assess the efficacy of KPL-387 | Time to Treatment Response by Week 24. Treatment |
| treatment in participants with | Response is defined as Pain Response (Numerical |
| recurrent pericarditis (RP). | Rating Scale [NRS] score ≤2 on the 11-point daily |
| | pericarditis pain NRS) and at least one C-reactive |
| | protein (CRP) level ≤0.5 mg/dL within 7 days before |
| | or after the Pain Response. |
| Secondary Objective - Part A | Secondary Efficacy Endpoints - Part A |
| To further assess the efficacy of | Time to Pain Response by Week 24. |
| KPL-387 treatment in participants | Time to CRP normalization (CRP ≤0.5 |
| with RP. | mg/dL) by Week 24. |
| | Percentage of days with no or minimal |
| | pericarditis pain (NRS ≤2) while on |
| | KPL-387 treatment over time through Week |
| | 24. No or minimal pain is defined as non- |
| | missing NRS score ≤2. |
| | Proportion of participants with normalization of |
| | CRP (CRP ≤0.5 mg/dL) over time through |
| | Week 24. |
| | Time to resolution of pericarditis manifestations |
| | (i.e. echocardiogram [ECHO] abnormalities, |
| | electrocardiogram [ECG] abnormalities, and |
| | pericardial rub), when present at baseline. |
| | Change from Baseline in SF-36v2 ® Physical |
| | Component Summary score through Week 24. |
| | Change from Baseline in SF-36v2 Mental |
| | Component Summary core through Week 24. |
| | Proportion of participants with Protocol- |
| | Defined Pericarditis Recurrence by Week 24. |
| | Change from Baseline in pericarditis pain |
| | NRS score through Week 24. |
| | Change from Baseline in CRP through Week 24. |
| Safety/PK Objective - Part A | Safety/PK Analyses - Part A |
| To assess the safety, tolerability, | Safety and Tolerability: |
| pharmacokinetics (PK), and | Treatment Emergent Adverse Events (TEAEs) |
| immunogenicity of KPL-387. | Clinical laboratory evaluations |
| | Vital sign measurements, |
| | electrocardiograms (ECGs), and physical |
| | examination findings |
| | PK parameters (e.g., Cₘₐₓ, tₘₐₓ, AUC₀₋ₜ, |
| | AUC₀₋ₜₐᵤ, AUC_{0-∞}, □Z, t_{1/2}, CL/F, V_{d}/F). |
| | Antidrug antibody (ADA) titers over time. |

### Objectives and Primary & Secondary Endpoints—Part B (Phase 3)

| Primary Objective - Part B | Primary Efficacy Endpoint | |
|---|---|---|
| To assess the efficacy of KPL-387 | Time to Pericarditis Recurrence, defined as the time | |
| treatment in participants with RP | from randomization in the Randomized Withdrawal | |
| | (RW) Period to the date of the first Pericarditis | |
| | Recurrence for each participant. | |
| | Only CEC-confirmed Pericarditis Recurrences will be | |
| | considered as events for the primary efficacy analysis | |
| | in Part B. | |
| Secondary Objective(s) - Part B | Secondary Efficacy Endpoint(s) - Part B | |
| To further assess the efficacy of | Secondary efficacy endpoints for the RI Period include: | |
| KPL-387 treatment in participants | | Time to Treatment Response by end of the RI |
| with RP. | | Period. Time to Treatment Response is defined as |
| | | the time from RI Baseline to the first day of Pain |
| | | Response (NRS ≤2 on the 11-point scale) and at |
| | | least 1 CRP level ≤0.5 mg/dL within 7 days |
| | | before or after the Pain Response. |
| | | Time to Clinical Response by end of RI Period. |
| | | Clinical Response is defined as the weekly average |
| | | of daily pericarditis pain score ≤2.0 on the 11-point |
| | | NRS and a CRP level ≤0.5 mg/dL, while receiving |
| | | study drug monotherapy, within the 7 days prior to |
| | | and including the day of assessment of Clinical |
| | | Response. |
| | | Time to Pain Response by end of RI Period. Time to |
| | | Pain Response is defined as time from RI Baseline |
| | | to the first day of daily pericarditis pain NRS ≤2. |
| | | Time to CRP normalization (CRP ≤0.5 mg/dL) by |
| | | end of RI Period. |
| | | Change from RI Baseline in SF-36v2 Physical |
| | | Component Summary score through end of RI |
| | | Period. |
| | | Change from RI Baseline in SF-36v2 Mental |
| | | Component Summary score through end of RI |
| | | Period. |
| | | Time to resolution of pericarditis manifestations |
| | | (i.e., ECHO abnormalities, ECG abnormalities, and |
| | | pericardial rub), when present at RI Baseline, by end |
| | | of RI Period. |
| | | Change from RI Baseline in daily pericarditis pain |
| | | NRS score through end of RI Period. |
| | | Change from RI Baseline in CRP through end of RI |
| | | Period. |
| | Secondary efficacy endpoints for the RW Period include: | |
| | | Proportion of participants who maintained |
| | | Clinical Response at RW Week 16. Clinical |
| | | Response is defined as the weekly average of |
| | | daily pericarditis pain on the 11-point NRS ≤2.0 |
| | | and CRP level ≤0.5 mg/dL, and on |
| | | monotherapy randomized study drug at RW |
| | | Week 16. |
| | | Percentage of days with no or minimal |
| | | pericarditis pain through RW Week 16. No or |
| | | minimal pericarditis pain is defined as non- |
| | | missing daily pericarditis pain NRS ≤2. |
| | | Change from RW Baseline in SF-36v2 Physical |
| | | Component Summary score at RW Week 16. |
| | | Change from RW Baseline in SF-36v2 Mental |
| | | Component Summary score at RW Week 16. |
| | | Change from RW Baseline in pain NRS |
| | | score during the RW Period. |
| | | Change from RW Baseline in CRP during the RW |
| | | Period. |
| Safety/PK Objective - Part B | | Safety/PK Endpoints - Part B |
| To assess the safety, tolerability, | | Safety and Tolerability: |
| pharmacokinetics (PK), and | | Treatment Emergent Adverse Events (TEAEs) |
| immunogenicity of KPL-387. | | Clinical laboratory evaluations |
| | | Vital sign measurements, electrocardiograms |
| | | (ECGs), and physical examination findings |
| | | KPL-387 serum concentrations over time. |
| | | Antidrug antibody (ADA) titers over time. |
| | | PK parameters (e.g., Cₘₐₓ, tₘₐₓ, AUC₀₋ₜ, |
| | | AUC₀₋ₜₐᵤ, AUC_{0-∞}, □Z, t_{1/2}, CL/F, V_{d}/F). |

### Objectives and Primary & Secondary Endpoints of Long-Term Extension (LTE; Part C)

| Primary Objective - Part C | Primary Efficacy Endpoint - Part C |
|---|---|
| To assess the long-term efficacy | Annualized rate of Pericarditis Recurrence through the |
| of KPL-387. | end of Part C (LTE). Only CEC-confirmed Pericarditis |
| | Recurrences will be considered as events for the |
| | analysis. |
| Secondary Objective - Part C | Secondary Efficacy Endpoints - Part C |
| To assess the long-term efficacy | Proportion of participants who maintained Clinical |
| of KPL-387. | Response through the end of the LTE. Clinical |
| | Response is defined as a weekly average of daily |
| | pericarditis pain on the 11-point NRS ≤2 and CRP |
| | level ≤0.5 mg/dL, while receiving KPL-387 |
| | monotherapy within the 7 days prior to and |
| | including the day of assessment of Clinical |
| | Response. |
| | Percentage of days with no or minimal pericarditis |
| | pain through the end of the LTE Period. No or |
| | minimal pericarditis pain is defined as non-missing |
| | daily pericarditis pain NRS ≤2. |
| | Change from LTE Baseline in SF-36v2 ® Physical |
| | Component Summary score through the end of the |
| | LTE Period. |
| | Change from LTE Baseline in SF-36v2 ® Mental |
| | Component Summary score through the end of the |
| | LTE Period. |
| | Change from the LTE Baseline in daily pericarditis |
| | pain NRS scores through the end of the LTE period. |
| Safety/PK Objectives - Part C | Safety/PK Endpoints - Part C |
| To assess the safety, tolerability, | Safety and Tolerability: |
| PK, and immunogenicity of KPL-387. | Treatment Emergent Adverse Events (TEAEs) |
| | Clinical laboratory evaluations |
| | Vital sign measurements, electrocardiograms |
| | (ECGs), and physical examination findings |
| | KPL-387 serum concentrations over time during the |
| | LTE Period. |
| | PK parameters (e.g., Cₘₐₓ, tₘₐₓ, AUC₀₋ₜ, AUC₀₋ₜₐᵤ, |
| | AUC_{0-∞}, lZ, t_{1/2}, CL/F, V_{d}/F). |
| | Antidrug antibody (ADA) titers over time during |
| | the LTE Period. |

### Study Design

This Phase 2/3 study comprised of three separate study parts: Part A, Part B, and Part C.

Part A is a dose-focusing evaluation of four different KPL-387 regimens (300 mg subcutaneous [SC] every two weeks [q2 wk], 300 mg SC every 4 weeks [q4 wk], 100 mg SC q2 wk, and 100 mg SC q4 wk). The totality of the data available from Part A will be used to confirm dose selection for Part B.

Part B will further evaluate efficacy and safety of the selected dose of KPL-387 in a blinded, placebo-controlled, randomized withdrawal study. Participants randomized in Part A are not eligible for participation in Part B.

Part C is a long-term extension (LTE) study to evaluate the long-term efficacy and safety of KPL-387 over time in participants continuing from both Part A and Part B.

### Part A Study Design:

Part A is a Phase 2, randomized, double-blind (participant and Investigator/site), multiple-dose, dose-focusing study to evaluate the dose response and safety of different KPL-387 dosing regimens.

To enroll in the study, all participants must meet predefined criteria for a Qualifying Pericarditis Episode. Eligible participants will be randomized to receive 1 of 4 different dose regimens of KPL-387 for treatment of up to 24 weeks. KPL-387 administration will be added to background pericarditis therapies (NSAIDs and/or colchicine) for assessment of Treatment Response, defined as a daily pericarditis pain NRS ≤2 and at least 1 CRP level ≤0.5 mg/dL within 7 days before or after the Pain Response. If there has been Treatment Response, all standard background treatments should be discontinued by Day 15 (Week 2 Visit) after study drug has been started; KPL-387 study drug will be administered as monotherapy or with sequential oral therapy (sORT) as needed for the remainder of the Treatment Period.

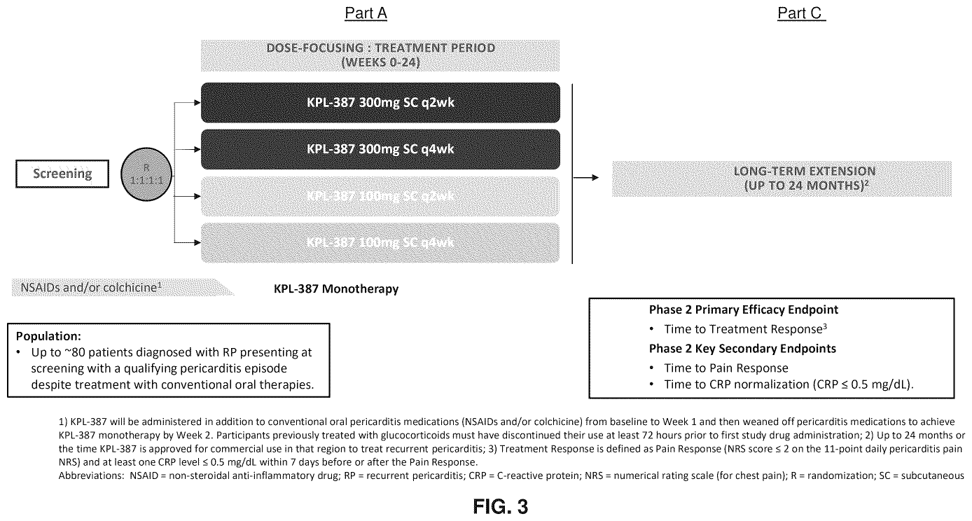

A study schematic for Part A (Phase 2) and Part C (LTE) is shown in FIG. 3.

### Part A Screening Period and Treatment Period

Participants will enter the study Screening Period of up to 4 weeks (Day −28 up to Day −1). During the Screening Period, screening laboratory evaluations, and clinical tests and evaluations will be performed, including assessment of disease characteristics. Qualifying NRS and CRP results must be confirmed within 7 days prior to the Baseline Visit. During the Screening Period, analgesics (including non-opioid and opioid) can be used for pain control. Glucocorticoids are permitted during the Screening Period, but participants must be able to discontinue these at least 72 hours prior to first study drug administration. NSAIDs and/or colchicine should be maintained at a stable dose for the 72 hours prior to the first study drug administration (and continued for the first 7 days of study drug treatment).

Participants will be randomized 1:1:1:1 to receive KPL-387 administered subcutaneously (300 mg SC q2 wk, 300 mg SC q4 wk, 100 mg SC q2 wk, or 100 mg SC q4 wk) during the 24-week Treatment Period. Participants and Investigators and site personnel will be blinded to the administered dose level and frequency. Study drug will be administered every two weeks; KPL-387 will be administered either every 2 weeks (q2 wk) or every 4 weeks (q4 wk). Participants receiving KPL-387 q4 wk will receive matching placebo on alternate dose administration days (at the 2-week interval) to maintain the blind.

The Treatment Period will consist of 3 periods:
Stabilization Period (Baseline and Week 1 Visit): During the Stabilization Period, KPL-387 will be administered in addition to Baseline concomitant pericarditis medications (NSAIDs and/or colchicine)
On-Treatment Weaning (After Week 1 Visit until Week 2 Visit): KPL-387 administration will continue as participants are weaned off background pericarditis therapies (i.e., NSAIDS and/or colchicine), as applicable, with the goal to achieve KPL-387 monotherapy by the Week 2 Visit (Day 15).
Q KPL-387 Monotherapy Treatment (After Week 2 Visit until Week 24 Visit): During the monotherapy treatment, participants will continue to receive study drug throughout the Treatment Period.

### Closure of Part A and Transition to Part C

Part B may be initiated before Part A is closed. Participants receiving KPL-387 in Part A are not eligible to participate in Part B, but participants in both Part A and Part B may be eligible to transition to Part C (LTE).

When the decision is made to close Part A, all active participants in Part A who have not completed an EOT Visit (i.e., are not in Safety Follow-Up), will be considered for eligibility to transition into Part C (LTE). Participants who are in the Treatment Period will return to the clinic or their next scheduled Part A Treatment Period visit, which will serve as both the Part A EOS Visit and as the Baseline Visit for Part C (LTE) participation. If a participant chooses not to participate in Part C (LTE), they will complete an EOS Visit (if >8 weeks since their last study drug dose) or an EOT Visit followed by a Safety Follow-Up until 8 weeks after their last dose of KPL-387.

### Part B Study Design

Part B is a Phase 3, double-blind (participant, Investigator/site, Sponsor), placebo-controlled, randomized, withdrawal study with long-term extension to evaluate efficacy and safety of KPL-387. Participants previously enrolled in Part A are not eligible for participation in Part B.

To be eligible for enrollment in Part B of the study, all participants must be experiencing a Qualifying Pericarditis Episode (defined previously).

During the Run-In (RI) Period participants will receive KPL-387; then, during which time KPL-387 administration will be added to background pericarditis therapies (NSAIDs and/or colchicine and/or glucocorticoids, in any combination) for assessment of Treatment Response (Pain Response and at least 1 CRP ≤0.5 mg/dL within 7 days before or after the Pain Response). All background standard treatments must be discontinued by the Week 8 Visit; KPL-387 will be administered as monotherapy for the remainder of the RI period.

All participants in the RI period will be blinded to the duration of the RI period and the timing of randomization. Participants who have achieved Clinical Response (defined as the weekly average of daily pericarditis pain score ≤2 on the 11-point NRS and a CRP level ≤0.5 mg/dL, while receiving study drug monotherapy, within the 7 days prior to and including the day of assessment of Clinical Response) throughout the RI Period will then be randomized 1:1 to receive KPL-387 or placebo during the RW Period. The RW Period will be closed after at least 22 CEC-confirmed Pericarditis Recurrence events have accrued and at least 40 participants have completed the RW Week 16 Visit or have received Bailout Treatment with KPL-387 prior to the RW Week 16 Visit.

Closure of the RW period will trigger the End of the Randomized Withdrawal (EORW) Visit for all participants in the RW Period. The EORW Visit should occur as soon as possible but a minimum of 14 days from the last study drug dose. The EORW Visit will also serve as the Part C (LTE) baseline for Part B participants in the RW Period who choose to participate in Part C (LTE).

Those participants in the RI Period at the time of closure of the RW Period will have a Part B EOS Visit and, at their choosing, either transition to the Part C LTE (if eligible based on the ability to have achieved Clinical Response on or before the end of the RI Period) as soon as possible but a minimum of 14 days from the last study drug dose or exit the study.

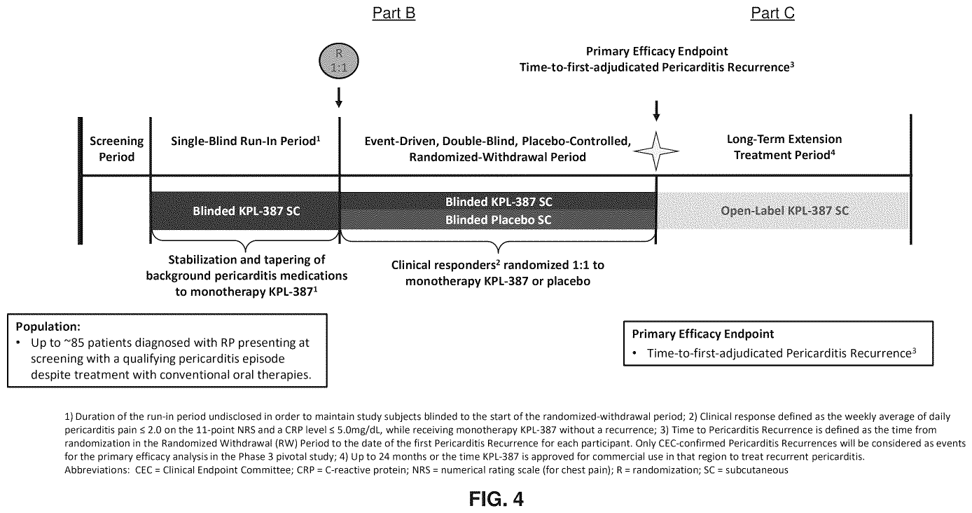

A study schematic for Part B (Phase 3) and Part C (LTE) is shown in FIG. 4.

### Part B Screening Period

Participants will enter the study Screening Period of up to 2 weeks (Day −14 up to Day −1).

During the Screening period, assessment of disease characteristics, baseline therapy, and the pretreatment clinical evaluation will be completed. During the Screening Period, analgesics (including non-opioid and opioid) can be used for pain control. Background pericarditis therapies should be maintained at a stable dose for at least 72 hours prior to first study drug administration. As appropriate, participants who screen fail may be rescreened once for this study with Medical Monitor approval. Rescreened participants will be assigned a new participant number.

### Part B Run-In (RI) Period

The planned dose level and frequency are KPL-387 300 mg SC q4 wk, pending confirmation in Part A

During the RI Period, all participants will initiate and receive single-blind KPL-387 and wean off background pericarditis medications to achieve KPL-387 monotherapy. Participants will be aware that they are receiving KPL-387, but they will not be aware of the duration of the Run-In period and the transition to the RW Period. Participants who have achieved Clinical Response, defined as the weekly average of daily pericarditis pain score ≤2 on the 11-point NRS and a CRP level 0.5 mg/dL, while receiving study drug monotherapy, within the 7 days prior to and including the day of assessment of Clinical Response, in the RI Period and are in Clinical Response, at the end of the RI Period will proceed into the double-blind, placebo-controlled RW Period. The RI Period will consist of the following:
Stabilization Period (RI Baseline through RI Week 1 Visit): KPL-387 will be administered in addition to background pericarditis therapies of NSAIDS, colchicine, and/or corticosteroids.
Weaning Period (After RI Week 1 Visit through RI Week 8 Visit): KPL-387 administration will continue as participants are weaned off background pericarditis therapies (i.e., NSAIDS, colchicine, and/or corticosteroids), as applicable, to achieve KPL-387 monotherapy at Week 8.
   Participants receiving NSAIDs and/or colchicine at Baseline will begin tapering/weaning at the RI Week 1 Visit and should complete withdrawal of these therapies by the RI Week 2 Visit (i.e., over a total of 1 week), but no later than RI Week 8.
   Participants receiving concomitant corticosteroids at Baseline will begin tapering/weaning at the RI Week 1 Visit with a goal of tapering off corticosteroid by the RI Week 8.
KPL-387 Monotherapy Period (After RI Week 8 Visit to end of RI Period): Participants who have successfully weaned off background pericarditis therapies and have achieved Clinical Response will continue to receive single-blind KPL-387 as a monotherapy through end of the RI Period. A minimum of 8 weeks of monotherapy (and completion of the RI Period) is required to move to the RW Period.

Participants who have not achieved/maintained Clinical Response at end of the RI Period will discontinue study drug, complete an EOT Visit, transition to pericarditis therapy according to local practice and at the Investigator's discretion, and complete their study participation with Safety Follow-Up until 8 weeks after their last administration of KPL-387.

### Part B Withdrawal (RW) Period

Participants who have achieved Clinical Response by RI Week 8 and maintained it at the end of the RI Period will proceed into the double-blind, placebo-controlled RW Period.

For the RW period, participants will be randomized 1:1 in a double-blind manner to receive KPL-387 300 mg Sc q4 wk (dose level and frequency pending confirmation in Part A) or matching placebo SC. Randomization will be stratified by (1) the Qualifying Pericarditis Episode representing the second or more recurrence after a symptom-free interval and (2) concomitant or prior corticosteroid or IL-1 pathway inhibitor use versus no prior steroid or IL-1 pathway inhibitor use (e.g., concomitant or prior NSAID and/or colchicine use).

The RW Period will be closed after at least 22 CEC-confirmed Pericarditis Recurrence events have occurred and at least 40 participants have completed the RW Week 16 Visit or have received bailout prior to the RW Week 16 Visit. For all active participants in the RW Period, including those who have received bail out, the End of RW (EORW) Visit will be scheduled as soon as possible, but no sooner than 14 days after the prior dose of study drug. The EORW Visit will also serve as the Part C (LTE) Baseline Visit for Part B participants in the RW Period who choose to participate in the Part C (LTE) of the study. Participants who choose not to participate in Part C (LTE) should also return for Safety Follow-Up until 8 weeks after their last dose of study drug.

### Closure of Part B and Transition to Part C

The RW Period will be closed after at least 22 CEC-confirmed Pericarditis Recurrences events have occurred and at least 40 participants have completed the RW Week 16 Visit or received bailout prior to the RW Week 16 Visit. For all active participants in the RW period (including those who have received bailout KPL-387), the EORW Visit will be scheduled as soon as possible, but no sooner than 14 days after the prior dose of study drug. The EORW Visit will also serve as the Part C (LTE) Baseline Visit for Part B participants in the RW Period who choose to participate in the Part C (LTE). Upon closure of Part B, participants in the RI Period that achieve Clinical Response at a clinic visit prior to or at the end of RI Period are eligible for LTE. These participants should complete the Part B RI EOS Visit (at least 14 days after last study drug dose), which will also serve as the Part C (LTE) Baseline Visit who choose to participate in the Part C (LTE). If a participant chooses not to participate in Part C (LTE), they will complete an EOT Visit followed by a Safety Follow-Up Visit 8 weeks after their last dose of KPL-387.

### Part C Study Design

Part C is an open-label, long-term extension study to evaluate the efficacy and safety of KPL-387. For the LTE study, participants will receive open-label KPL-387. Treatment in Part C (LTE) may continue until the participant has received 24 months of treatment in the LTE or KPL-387 is approved for commercial use to treat pericarditis. Exemplary schematics for transitioning to the LTE study from Parts A and B are shown in FIG. 3 and FIG. 4, respectively.

Active, enrolled participants from Part A who have not completed an EOT Visit may be eligible to participate in Part C. All active RW participants may be eligible to transition to Part C. All active RI participants who have achieved Clinical Response on or before end of the RI Period may be eligible to transition to Part C.

### Study Population:

Participants in this study must have a clinical diagnosis of pericarditis based on the 2015 European Society of Cardiology (ESC) Guidelines for the Diagnosis and Management of Pericardial Diseases (Adler 2015) definition.

Eligible participants are those presenting at Screening with a Qualifying Pericarditis Episode, consisting of symptoms of acute pericarditis (i.e., moderate to severe chest pain) accompanied by elevated serum C-reactive protein (CRP) despite treatment with standard oral therapies, that represents either: (1) at least the second pericarditis recurrence (i.e., at least the third pericarditis episode), with the initial acute pericarditis episode having been followed by a symptom-free interval, or (2) persistent pericarditis signs and symptoms despite initial improvement without a symptom-free interval.

To provide sufficient sensitivity and dynamic range for study endpoints, a Qualifying Pericarditis Episode is defined as at least 1 day with pericarditis pain measurement ≥4 on the 11-point pericarditis pain Numerical Rating Scale (NRS) and at least 1 CRP local or central laboratory-reported value ≥1 mg/dL, from a sample drawn within 7 days before or after the qualifying NRS.

In Part A, the dose-focusing Phase 2 portion of the study, participants may qualify while receiving concomitant non-steroidal anti-inflammatory drugs (NSAIDs) and/or colchicine (but not glucocorticoids), provided that the dosages of NSAID and/or colchicine have been stable for at least 72 hours prior to first study drug administration and that, in the opinion of the Investigator, these medications can likely be weaned off during the weaning period (between Week 1 Visit and Week 2 Visit). Participants previously treated with glucocorticoids must have discontinued their use at least 72 hours prior to first study drug administration. Concomitant glucocorticoids use is not permitted in Part A.

In Part B, the Phase 3 portion of the study, participants may qualify while receiving concomitant NSAIDs and/or colchicine and/or oral glucocorticoids (in any combination), provided that the dosages of these medications have been stable for at least 72 hours prior to first study drug administration and that, in the opinion of the Investigator, these therapies can likely be weaned off during the weaning period (Week 1 to Week 8).

In Part C (LTE), only participants from either Part A or Part B who are active in the study at the time Part C is initiated will be considered for eligibility to participate (i.e., participants who have discontinued for any reason will be ineligible).

### Inclusion Criteria

1. Is capable of understanding the written ICF, has provided signed written informed consent, and agrees to comply with protocol requirements.
2. Is ≥18 and ≤81 years of age.
3. Has a body weight of at least 40 kg (88.2 lbs) at Screening.
4. Has a history, or current clinical diagnosis, of pericarditis that, in the opinion of the Investigator, is consistent with the 2015 ESC Guidelines for the Diagnosis and Management of Pericardial Diseases (Adler 2015), based on the documented available data, fulfilling at least 2 of the following 4 criteria:
   a. Pericarditic chest pain
   b. Pericardial rub
   c. New widespread ST segment elevation or PR segment depression according to ECG findings
   d. Pericardial effusion (new or worsening)
5. Presents at Screening with signs and symptom of acute pericarditis (Qualifying Pericarditis Episode) despite treatment with standard therapies that represents either:
   a. At least the second recurrence (i.e., at least the third pericarditis episode), with the initial acute pericarditis episode having been followed by a symptom-free interval; OR
   b. Persistent pericarditis signs and symptoms despite initial improvement without a symptom-free interval.
6. Qualifying Pericarditis Episode is confirmed by the presence of both:
   a. At least 1 day with pericarditis pain ≥4 on the 11-point NRS, AND
   b. At least 1 CRP level ≥1.0 mg/dL drawn within 7 days before or after the qualifying NRS.
      Qualifying Pericarditis Episode should have occurred within the 7 days prior to Part A Treatment Baseline Visit or Part B RI Baseline Visit (first study drug administration). Pericarditis pain (NRS ≥4) and CRP ≥1 mg/dL are not required to be present on the same day.
7. Pericarditis medications at Baseline:
   a. Part A: NSAIDs and/or colchicine (in any combination), if used, have been maintained at stable dose levels (not increased) for at least 72 hours prior to Baseline (first study drug administration), and changes in medications made within this time period (e.g., 1-time use of NSAIDs) are not anticipated to significantly alter assessments of baseline disease activity in the opinion of the Investigator.
      Concomitant glucocorticoid use is not permitted in Part A, and prior use must have been discontinued at least 72 hours prior to first study drug administration.
   b. Part B: NSAIDs and/or colchicine and/or glucocorticoids (in any combination), if used, have been at stable dose levels (not increased) for at least 72 hours prior to RI Baseline (first study drug administration), and changes in medications made within this time period (e.g., 1-time use of NSAIDs) are not anticipated to significantly alter assessments of baseline disease activity in the opinion of the Investigator.
8. Participant is willing to and, in the opinion of the Investigator, is expected to be able to discontinue all permitted prior/concomitant pericarditis medications after initiation of study drug within the protocol-specified windows.
9. Routine adult vaccinations should be up to date and/or offered at least 2 weeks prior to randomization according to regional and national guidelines based on medical history or presence of risk factors, in the opinion of the Investigator.
10. If female, must be either postmenopausal (defined as no menses for 12 months without other medical cause), permanently surgically sterile (i.e., removal of ovaries, fallopian tubes, and/or uterus), or, for women of child-bearing potential, must:
   Be nonpregnant, nonlactating, and agree to remain abstinent or use a highly effective method of contraception with a failure rate of <1% per year from the Screening Visit until the end of study participation and at least 8 weeks after last study drug dose. Examples of contraception methods with a failure rate of <1% per year include:
      Hormonal contraceptives associated with inhibition of ovulation (stable dose for at least 4 weeks prior to first dose of study drug); hormonal contraceptive methods must be supplemented by a barrier method
      Hormone-releasing or copper intrauterine device
      Other intrauterine system
      Bilateral tubal occlusion
      Vasectomized male partner
      Abstinence from heterosexual intercourse; The reliability of sexual abstinence should be evaluated based on duration of the study and usual lifestyle of the participant. Intermittent abstinence (such as calendar, ovulation, symptothermal or postovulation methods) and withdrawal are not considered acceptable contraceptive methods.
   The definition of childbearing potential may be adapted for alignment with local guidelines or regulations.
11. Sexually active male participants must have documented vasectomy or must agree to use a method of contraception with a failure rate of <1% per year (as defined above) with their partners of childbearing potential through the end of study participation and at least 8 weeks after last study drug dose.
12. Male participants must agree to refrain from donating sperm through the end of study participation and at least 8 weeks after last study drug dose. Female participants must agree to refrain from donating eggs through the end of study participations and at least 8 weeks after last study drug dose.
13. Agrees to refrain from lifestyle changes that may affect pericarditis symptoms (e.g., significant changes to exercise patter) except as recommended by the Investigator from the time the ICF is signed through the end of Part A or Part B.

### Criteria for Evaluation

### Efficacy

A brief description of exemplary efficacy assessments are provided below.

11-Point Pericarditis Pain Numerical Rating Scale (NRS): Participants will be asked to provide a daily assessment of pericarditis pain. A validated 11-point pericarditis NRS will be used to measure the level of pain intensity daily throughout the study (Hawker 2011; Mannion 2007; Dworkin 2005). It is recommended that daily pericarditis pain measurements using the 11-point pericarditis NRS are performed at the same time of the day each day, and prior to study drug injection on days with study drug administration. Participants will complete the NRS at home and will log their score daily into a study-provided electronic diary.

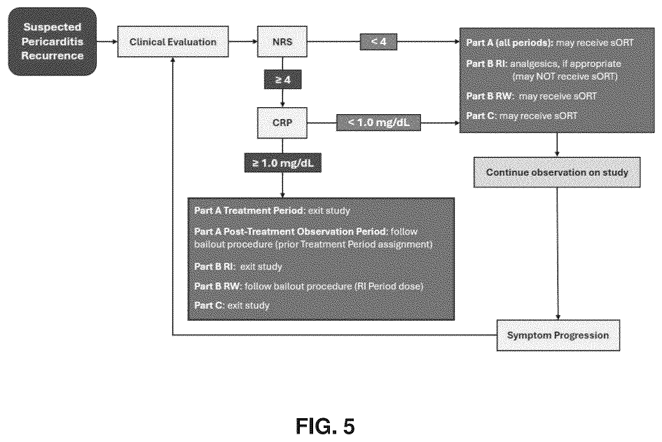

C-Reactive Protein (CRP): Central and local laboratory assessments of CRP will be performed at study visits. Centrally assayed CRP values will be used for statistical analyses. Locally assayed CRP will be used for screening, evaluation of Treatment Response, and assessment and management of Suspected Pericarditis Recurrence, as exemplified in FIG. 5.

Cytokine IL-6: Centrally assayed IL-6 values will be measured in real time and may be used for statistical evaluations of changes from baseline.

Electrocardiogram: Electrocardiographic assessment of ECG abnormalities will be obtained. Pericarditis commonly involves changes in the electrophysiologic activity of the heart, resulting in typical ECG findings, namely widespread ST segment elevation or PR segment depression.

Echocardiogram: Echocardiographic assessment of presence of pericardial effusion including new or worsening pericardial effusion will be obtained. Pericardial effusion is characterized by accumulation of excess fluid in the pericardial space surrounding the heart, and pericarditis is one of the main causes of pericardial effusion. Echocardiography is a sensitive tool and the most widely used imaging technique for the detection of pericardial effusion and/or thickening. Echocardiograms are to be acquired per the ECHO core imaging parameters. Results may be evaluated by local assessment and interpretation. ECHO imaging and data will also be submitted to the designated central imaging laboratory for central assessment and interpretation.

36-Item Short Form Health Survey (SF-36v2®): The SF-36 is a set of generic, coherent, and easily administered quality of life measures. The SF-36 assesses 8 health domains: physical functioning, bodily pain, role limitations due to physical health problems, role limitations due to personal or emotional problems, general mental health, social functioning, energy/fatigue, and general health perceptions. It also includes a single item that provides an indication of perceived change in health.

EQ-5D-5L: The EQ-5D-5L is a standardized instrument developed by the EuroQol Group as a measure of health-related quality of life that can be used in a wide range of health conditions and treatments. EQ-5D-5L consists of a descriptive system and the EQ visual analog scale (VAS). The descriptive system comprises 5 dimensions: mobility, self-case, usual activities, pain/discomfort, and anxiety/depression. The rating scale records the participant's self-rated health on a vertical VAS. This can be used as a quantitative measure of health outcome that reflects the participant's own judgement. The scores on these 5 dimensions can be presented as a health profile or can be converted to a single summary index number (utility) reflecting preferability compared to other health profiles.

### Safety

Safety assessments to be performed at study visits and include assessment of AEs, physical examinations (full and abbreviated), vital signs measurements, chest x-ray, and clinical laboratory tests (e.g., serology, hematology, chemistry, urinalysis). A serum pregnancy test will be conducted at Screening. Urine pregnancy tests for participants of childbearing potential will be conducted as needed during the study. Local tolerability at the injections site will also be evaluated by a trained observer at every clinic visit.

### Example 4. KPL-387 is Effective in Treating a Patient with Recurrent Pericarditis

This example demonstrates the efficacy of KPL-387 in treating a representative patient with recurrent pericarditis.

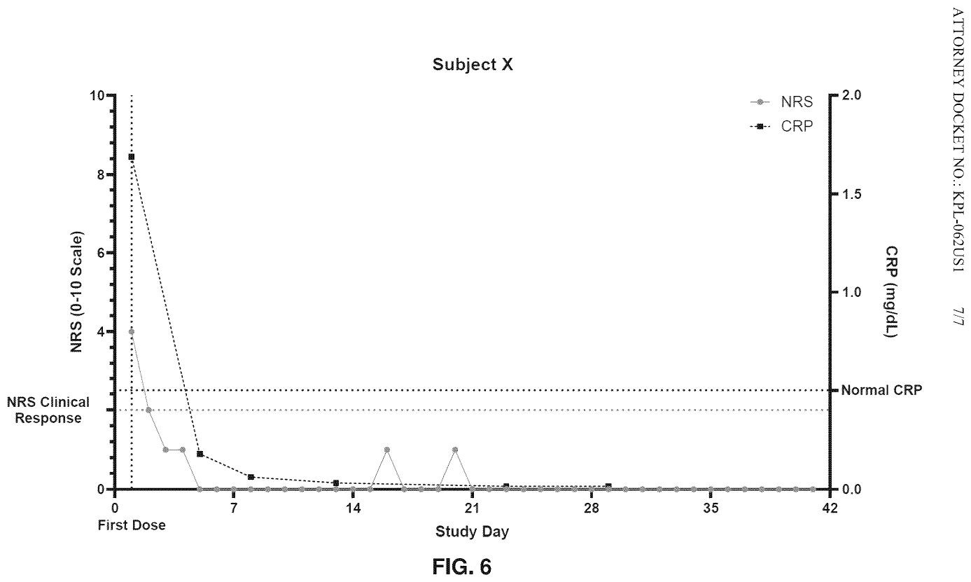

Subject X was administered the first dose KPL-387 on Day 1. The subject's NRS score and CRP levels were determined periodically and shown in FIG. 6. As shown, administration of KPL-387 elicited a treatment response in reducing Subject X's NRS scores and reduced CRP levels to within the normal range within 1 week of the first dose. Overall, KPL-387 was effective in treating signs and symptoms of an acute pericarditis recurrence in Subject X.

### EQUIVALENTS AND SCOPE

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

1. A method of treating recurrent pericarditis comprising administering to a patient, an anti-interleukin-1 receptor type-1 (IL1R1) antibody,
wherein the anti-IL1R1 antibody comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6.

2. The method of claim 1, wherein the anti-IL1R1 comprises a VH of SEQ ID NO: 7.

3. The method of claim 1, wherein the anti-IL1R1 comprises a VL of SEQ ID NO: 8.

4. The method of claim 1, wherein the anti-IL1R1 comprises a VH of SEQ ID NO: 7, and a VL of SEQ ID NO: 8.

5. The method of claim 4, wherein the anti-IL1R1 antibody comprises a heavy chain of SEQ ID NO: 10 and a light chain of SEQ ID NO: 11

6. The method of claim 1, wherein the anti-IL1R1 antibody is administered subcutaneously.

7. The method of claim 1, wherein the administration of the anti-IL1R1 antibody results in a reduced CRP value of ≤1.0 mg/dL.

8. The method of claim 7, wherein the administration of the anti-IL1R1 antibody results in a reduced CRP value of ≤0.5 mg/dL.

9. The method of claim 8, wherein the reduced CRP value of ≤0.5 mg/dL is achieved within 1 week from the first administration of the anti-IL1R1 antibody.

10. The method of claim 1, wherein the administration of the anti-IL1R1 antibody results in NRS score of 2 or less.

11. The method of claim 10, wherein the NRS score of 2 or less is achieved within 1 week from the first administration of the anti-IL1R1 antibody.

12. The method of claim 1, wherein the recurrent pericarditis is idiopathic recurrent pericarditis.


SEQUENCE LISTING (66 sequences)

SEQ ID NO: 1 - protein, 5 aa, synthetic construct
FHWIA

SEQ ID NO: 2 - protein, 17 aa, synthetic construct
IIHPGASDTRYSPSFQG

SEQ ID NO: 3 - protein, 9 aa, synthetic construct
QRELDYFDY

SEQ ID NO: 4 - protein, 11 aa, synthetic construct
RASQSIGSSLH

SEQ ID NO: 5 - protein, 7 aa, synthetic construct
YASQSFS

SEQ ID NO: 6 - protein, 9 aa, synthetic construct
HQSSSLPLT

SEQ ID NO: 7 - protein, 118 aa, synthetic construct
EVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSS

SEQ ID NO: 8 - protein, 107 aa, synthetic construct
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIK

SEQ ID NO: 9 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 444 aa, synthetic construct
EVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 214 aa, synthetic construct
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 7 aa, synthetic construct
GYSFSFH

SEQ ID NO: 13 - protein, 6 aa, synthetic construct
HPGASD

SEQ ID NO: 14 - protein, 8 aa, synthetic construct
GYSFSFHW

SEQ ID NO: 15 - protein, 8 aa, synthetic construct
IHPGASDT

SEQ ID NO: 16 - protein, 6 aa, synthetic construct
QSIGSS

SEQ ID NO: 17-18 - skipped (2 entries)

SEQ ID NO: 19 - protein, 11 aa, synthetic construct
ARQRELDYFDY

SEQ ID NO: 20 - protein, 5 aa, synthetic construct
SYAMS

SEQ ID NO: 21 - protein, 17 aa, synthetic construct
AISGSGGSTYYADSVKG

SEQ ID NO: 22 - protein, 17 aa, synthetic construct
DGASSTNWGYNYYGMDV

SEQ ID NO: 23 - protein, 13 aa, synthetic construct
SGSSSNIGSDYVF

SEQ ID NO: 24 - protein, 7 aa, synthetic construct
WNNQRPS

SEQ ID NO: 25 - protein, 11 aa, synthetic construct
AAWDDSLSGLV

SEQ ID NO: 26 - protein, 17 aa, synthetic construct
DGASSTNWGYTVDAAVD

SEQ ID NO: 27 - protein, 11 aa, synthetic construct
AAWDDHLEQLH

SEQ ID NO: 28 - protein, 17 aa, synthetic construct
DGASSTNWGYTLDPPGV

SEQ ID NO: 29 - protein, 12 aa, synthetic construct
AAWDDAAARVLL

SEQ ID NO: 30 - protein, 18 aa, synthetic construct
PLYYYDGSDYTTYDAFDI

SEQ ID NO: 31 - protein, 14 aa, synthetic construct
TGSSSNIGAGYDVH

SEQ ID NO: 32 - protein, 7 aa, synthetic construct
GDTHRPS

SEQ ID NO: 33 - protein, 11 aa, synthetic construct
QSYDTSLSGSL

SEQ ID NO: 34 - protein, 17 aa, synthetic construct
PLYYYDQQYGVVYDAFG

SEQ ID NO: 35 - protein, 10 aa, synthetic construct
QSYDVRLHHV

SEQ ID NO: 36 - protein, 17 aa, synthetic construct
PLYYYDQQYGVVYDAFV

SEQ ID NO: 37 - protein, 18 aa, synthetic construct
PLYYYDAAPPLGYDGFDI

SEQ ID NO: 38 - protein, 11 aa, synthetic construct
QSYDTDAARHQ

SEQ ID NO: 39 - protein, 18 aa, synthetic construct
PLYYYDAPSPLGYDGFDI

SEQ ID NO: 40 - protein, 11 aa, synthetic construct
QSYDTHLVAHV

SEQ ID NO: 41 - protein, 18 aa, synthetic construct
PLYYYDEQYGLVYDAFDI

SEQ ID NO: 42 - protein, 11 aa, synthetic construct
QSYDTLLLAPQ

SEQ ID NO: 43 - protein, 18 aa, synthetic construct
PLYYYDESLALPVYDADI

SEQ ID NO: 44 - protein, 11 aa, synthetic construct
QSYDTRADDAH

SEQ ID NO: 45 - protein, 13 aa, synthetic construct
SGSSSNIGSNYVF

SEQ ID NO: 46 - protein, 11 aa, synthetic construct
QSYDTAGGGHH

SEQ ID NO: 47 - protein, 5 aa, synthetic construct
SYMAS

SEQ ID NO: 48 - protein, 11 aa, synthetic construct
QSYDTVRLHHV

SEQ ID NO: 49 - protein, 5 aa, synthetic construct
SNSIT

SEQ ID NO: 50 - protein, 16 aa, synthetic construct
MIWSNGDTDYNSAFTS

SEQ ID NO: 51 - protein, 7 aa, synthetic construct
YNYYFDY

SEQ ID NO: 52 - protein, 15 aa, synthetic construct
KASQNVDNRGVSYVH

SEQ ID NO: 53 - protein, 7 aa, synthetic construct
KGSNLAF

SEQ ID NO: 54 - protein, 9 aa, synthetic construct
QQSKGHPDT

SEQ ID NO: 55 - protein, 5 aa, synthetic construct
NYGMH

SEQ ID NO: 56 - protein, 17 aa, synthetic construct
GIWNDGINKYHAHSVRG

SEQ ID NO: 57 - protein, 11 aa, synthetic construct
ARSFDWLLFEF

SEQ ID NO: 58 - protein, 11 aa, synthetic construct
RASQSVSSYLA

SEQ ID NO: 59 - protein, 7 aa, synthetic construct
DASNRAT

SEQ ID NO: 60 - protein, 10 aa, synthetic construct
QQRSNWPPLT

SEQ ID NO: 61 - protein, 17 aa, synthetic construct
AIWNDGENKHHAGSVRG

SEQ ID NO: 62 - protein, 11 aa, synthetic construct
GRYFDWLLFEY

SEQ ID NO: 63 - protein, 221 aa, synthetic construct
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 64 - protein, 247 aa, synthetic construct
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 65 - protein, 577 aa, synthetic construct
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 327 aa, synthetic construct
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC